(19) United States
(12) Patent Application Publication
(10) Pub. No.: US 2023/0329090 A1
(43) Pub. Date: Oct. 12, 2023

(54) SPIRO COMPOUND AND APPLICATION THEREOF

(71) Applicant: SICHUAN AG-RAY NEW MATERIALS CO., LTD, Meishan City, Sichuan (CN)

(72) Inventors: Liangliang YAN, Foshan (CN); Shaofu CHEN, Foshan (CN); Lei DAI, Foshan (CN); Lifei CAI, Foshan (CN)

(73) Assignee: SICHUAN AG-RAY NEW MATERIALS CO., LTD, Meishan City, Sichuan (CN)

(21) Appl. No.: 18/020,916

(22) PCT Filed: Jun. 11, 2022
(86) PCT No.: PCT/CN2022/098281

§ 371 Date: Feb. 10, 2023

(30) Foreign Application Priority Data
Jul. 1, 2021 (CN) 202110746027.5
Jun. 2, 2022 (CN) 202210619940.3

(51) Int. Cl. H10K 85/60; C07D 307/91; C07D 309/04; C07C 211/61; C07D 209/86; C07D 307/14; C09K 11/06

(52) U.S. Cl. CPC H10K 85/633; C07C 211/61; C07D 209/86; C07D 307/14; C07D 307/91; C07D 309/04; C09K 11/06; H10K 85/636; H10K 50/15

(57) ABSTRACT

The present disclosure relates to a spiro compound and application thereof. The spiro compound has a structure as shown in a formula (1). The material provided in the present disclosure has advantages such as high optical and electrical stability, low sublimation temperature, low drive current, low lateral mobility of carriers, high luminous efficiency, and long service life of a device, and can be used in an organic electroluminescent device. In particular, the compound has the possibility of being applied in the AMOLED industry as a hole injection or transport material.

### TECHNICAL FIELD

The present disclosure relates to the technical field of organic electroluminescence, in particular to an organic light-emitting material applicable to organic electroluminescent devices, and specially in particular to a spiro compound and application thereof.

### BACKGROUND

At present, as a new-generation display technology, an organic electroluminescent device (OLED) has attracted more and more attention in display and lighting technologies, thus having a wide application prospect. However, compared with market application requirements, properties, such as luminous efficiency, driving voltage, and service life of OLED devices still need to be strengthened and improved.

In generally, the OLED devices include various organic functional material films with different functions sandwiched between metal electrodes as basic structures, which are similar to sandwich structures. Under the driving of a current, holes and electrons are injected from a cathode and an anode, respectively. After moving a certain distance, the holes and the electrons are compounded in a light-emitting layer, and then released in the form of light or heat to achieve luminescence of the OLED. However, organic functional materials are core components of the OLED devices, and the thermal stability, photochemical stability, electrochemical stability, quantum yield, film forming stability, crystallinity, and color saturation of the materials are main factors affecting properties of the devices.

In order to obtain organic light-emitting devices with excellent properties, the selection of materials is particularly important. Not only is an emitter material having a light-emitting effect included, but also a hole injection material, a hole transport material, a main material, an electron transport material, an electron injection material and other functional materials that are mainly used for injection and transportation of carriers in the devices are included. Through selection and optimization of the materials, the transportation efficiency of holes and electrons can be improved, and the holes and the electrons in the devices can reach a balance, so that the voltage, luminous efficiency, and service life of the devices are improved.

According to a patent document 1 (CN103108859B), a spirofluorene aromatic amine with a structure of

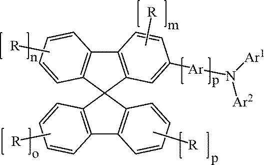

used as a hole transport material is recorded. On the basis of the material, good properties of a device are provided. However, the service life of a device, especially the service life of a blue light-emitting device, is required to be further improved. In addition, the lateral hole mobility of the material is also required to be further improved, so as to provide OLED products with good low gray-scale color purity. According to a patent document 2 (CN103641726B), a spirofluorene aromatic amine with a structure of

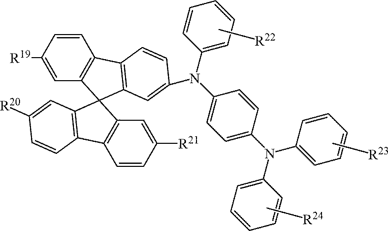

used as a second hole transport material is recorded. On the basis of the material, properties of a device are required to be greatly improved, especially the efficiency of a device. According to a patent document 3 (CN111548278A), a spirofluorene aromatic amine with a structure of

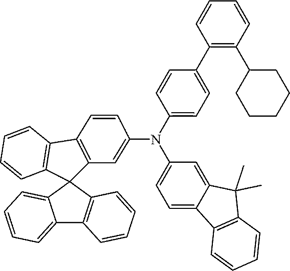 [SMILES: CC1(C)c2ccccc2-c2ccc(N(c3ccc(-c4ccccc4C4CCCCC4)cc3)c3ccc4c(c3)C3(c5ccccc5-c5ccccc53)c3ccccc3-4)cc21]

used as a hole transport material in which an aromatic amine includes substituents such as alkyl, deuterium, and cycloalkyl is recorded. On the basis of the material, properties of a device are required to be further improved, especially the service life of a device. According to a non-patent document 1 (J. Mater. Chem., 2005, 15,2455-2463) by Jiun Yi Shen et al., a blue light-emitting material with a spirofluorene structure as a basic construction, such as

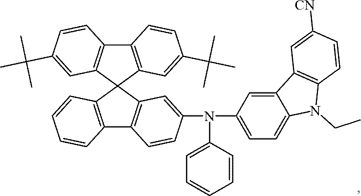

is disclosed. When the material is used as a blue light-emitting layer, the luminous efficiency and service life of a device are required to be improved. In addition, when the material is used as a hole transport material, the same problems also exist and are required to be optimized and improved.

### SUMMARY

In order to solve the above defects, the present disclosure provides an organic electroluminescent device with high properties and a spiro compound material capable of realizing the organic electroluminescent device.

The spiro compound of the present disclosure has a structure as shown in a formula (1). The spiro compound provided in the present disclosure has advantages such as high optical and electrical stability, low sublimation temperature, low drive current, low lateral mobility of carriers, high luminous efficiency, and long service life of a device, and can be used in an organic electroluminescent device. In particular, the compound has the possibility of being applied in the AMOLED industry as a hole injection or transport material.

A spiro compound has a structure as shown in a formula (1),

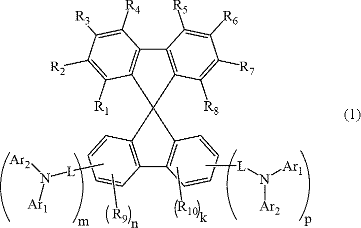

where R₁-R₁₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀ heteroalkyl, substituted or unsubstituted C₃-C₂₀ cycloalkyl, substituted or unsubstituted C₃-C₂₀ heterocyclic alkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀ alkyl silyl, substituted or unsubstituted tri-C₆-C₁₂ aryl silyl, substituted or unsubstituted di-C₁-C₁₀ alkyl mono-C₆-C₃₀ aryl silyl, and substituted or unsubstituted mono-C₁-C₁₀ alkyl di-C₆-C₃₀ aryl silyl, or two adjacent groups of R₁-R₈ and R₉-R₁₀ may be connected to each other to form an aliphatic ring or an aromatic ring structure;
at least two groups of the R₁-R₈ are substituted or unsubstituted C₃-C₂₀ cycloalkyl, or substituted or unsubstituted C₃-C₂₀ heterocyclic alkyl;
L is independently selected from a single bond, substituted or unsubstituted C₆-C₃₀ arylene, or substituted or unsubstituted C₂-C₃₀ heteroarylene;
Ar₁ and Ar₂ are independently selected from substituted or unsubstituted C₆-C₃₀ aryl, or substituted or unsubstituted C₂-C₃₀ heteroaryl;
m, n, h, and p are independently selected from 0 or an integer of 1-4, m+n=4, p+k=4, and the m and the p are not 0 at the same time;
the heteroalkyl and the heteroaryl at least contain one O, N, or S heteroatom; and
the “substituted” refers to substitution with deuterium, F, Cl, Br, C₆-C₁₀ aryl, C₁-C₆ alkyl, C₃-C₆ cycloalkyl, amino substituted with C₁-C₆ alkyl, cyano, isonitrile, or phosphino, and the substitution number ranges from a single substitution number to a maximum substitution number.

As a preferred spiro compound, m+p=1.

As a preferred spiro compound, the spiro compound has structures as shown in a formula (2) to a formula (9),

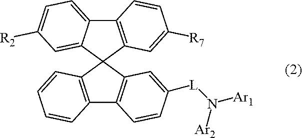

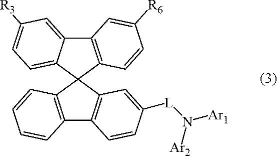

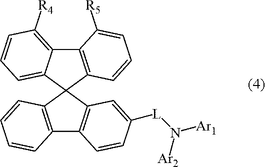

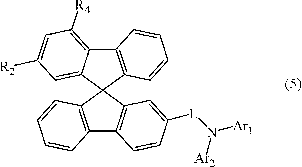

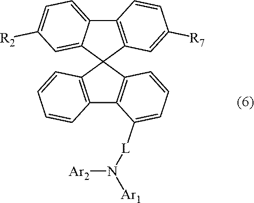

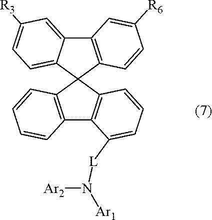

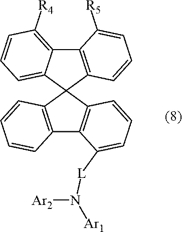

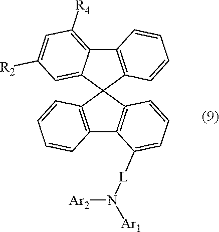

where R₂, R₃, R₄, R₅, R₆, and R₇ are substituted or unsubstituted C₃-C₂₀ cycloalkyl, or substituted or unsubstituted C₃-C₂₀ heterocyclic alkyl; and
other symbols are defined the same as above.

As a preferred spiro compound, the spiro compound has a structure as shown in the formula (2) or formula (6), the R₂ and the R₇ are the same or different, and Ar₁ and Ar₂ are the same or different.

As a preferred spiro compound, L in the formula (2) to the formula (9) is preferably a single bond.

As a preferred spiro compound, the spiro compound preferably has structures as shown in a formula (10) to a formula (11),

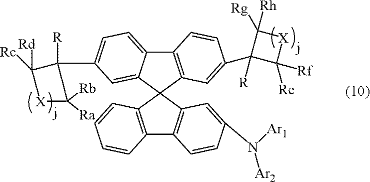

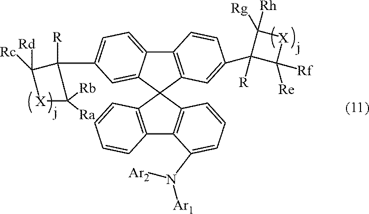

where X is independently selected from C(R₀)₂, 0, S, and NR₀;
j is independently 0 or an integer of 1-7; when the j is equal to 0, a ring formed is a ternary ring; when the j is equal to or greater than 2, various kinds of the X are the same or different;
R, R₀, and Ra-Rh are independently selected from hydrogen, deuterium, halogen, cyano, hydroxyl, sulfhydryl, amino, substituted or unsubstituted C₁-C₁₀ alkyl, substituted or unsubstituted C₁-C₁₀ heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀ alkyl silyl, substituted or unsubstituted tri-C₆-C₁₂ aryl silyl, substituted or unsubstituted di-C₁-C₁₀ alkyl mono-C₆-C₃₀ aryl silyl, and substituted or unsubstituted mono-C₁-C₁₀ alkyl di-C₆-C₃₀ aryl silyl, or four groups of Ra, Rb, Rc, and Rd and/or four groups of Re, Rf, Rg, and Rh and/or various kinds of the R₀ and/or the R and other substituents are connected to each other to form a ring structure; and
the “substituted” refers to substitution with deuterium, F, Cl, Br, C₆-C₁₀ aryl, C₁-C₆ alkyl, C₃-C₆ cycloalkyl, amino substituted with C₁-C₆ alkyl, cyano, isonitrile, or phosphino, and the substitution number ranges from a single substitution number to a maximum substitution number.

The R is hydrogen, deuterium, substituted or unsubstituted C₁-C₁₀ alkyl, or substituted or unsubstituted C₁-C₁₀ heteroalkyl; and

the R₀ and the Ra-Rh are independently selected from hydrogen, deuterium, halogen, substituted or unsubstituted C₁-C₁₀ alkyl, substituted or unsubstituted C₁-C₁₀ heteroalkyl, and substituted or unsubstituted C₃-C₂₀ cycloalkyl, or four groups of the Ra, the Rb, the Rc, and the Rd and/or four groups of the Re, the Rf, the Rg, and the Rh and/or various kinds of the R₀ are connected to each other to form a ring structure

As a preferred spiro compound, the R is preferably hydrogen, deuterium, substituted or unsubstituted C₁-C₁₀ alkyl, or substituted or unsubstituted C₁-C₁₀ heteroalkyl.

As a preferred spiro compound, the j is preferably a value equal to or greater than 2.

As a preferred spiro compound, at most one of 2 or more of the X is O, S, Se, or NR₀.

As a preferred spiro compound, various kinds of the R₀ and/or the R and the R₀ are preferably connected to each other to form a ring structure.

The R₂ and the R₇ are the same, and the Ar₁ and the Ar₂ are different; and the Ar₁ and the Ar₂ are independently selected from substituted or unsubstituted phenyl, biphenyl, naphthyl, fluorenyl, dibenzofuranyl, or carbazolyl, and the “substituted” refers to substitution with deuterium, F, Cl, Br, C₆-C₁₀ aryl, C₁-C₆ alkyl, or C₃-C₆ cycloalkyl.

As a preferred spiro compound, the spiro compound preferably has one of the following structural formulas, or is partially or completely deuterated or fluorinated correspondingly,

| | | | |
|---|---|---|---|
| 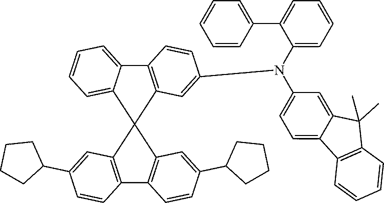 [SMILES: CC1(C)c2ccccc2-c2ccc(N(c3ccc4c(c3)C3(c5ccccc5-4)c4cc(C5CCCC5)ccc4-c4ccc(C5CCCC5)cc43)c3ccccc3-c3ccccc3)cc21] | 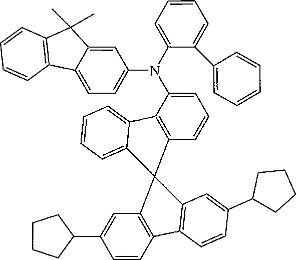 [SMILES: CC1(C)c2ccccc2-c2ccc(N(c3ccccc3-c3ccccc3)c3cccc4c3-c3ccccc3C43c4cc(C5CCCC5)ccc4-c4ccc(C5CCCC5)cc43)cc21] | 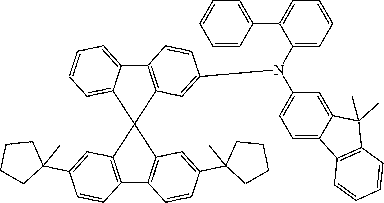 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCCC4)ccc2-3)CCCC1] | 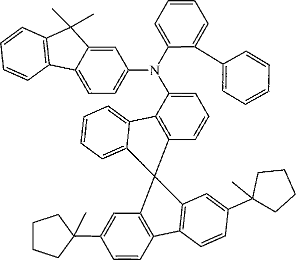 [SMILES: CC1(c2ccc3c(c2)C2(c4cc(C5(C)CCCC5)ccc4-3)c3ccccc3-c3c(N(c4ccc5c(c4)C(C)(C)c4ccccc4-5)c4ccccc4-c4ccccc4)cccc32)CCCC1] |
| CPD001 | CPD002 | CPD003 | CPD004 |
| 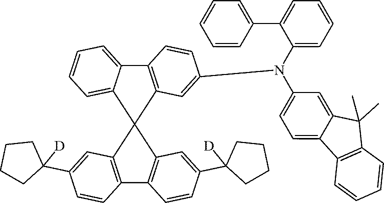 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCCC4)ccc2-3)CCCC1] | 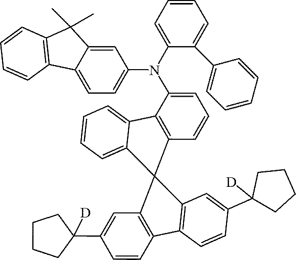 [SMILES: [2H]C1(c2ccc3c(c2)C2(c4cc(C5([2H])CCCC5)ccc4-3)c3ccccc3-c3c(N(c4ccc5c(c4)C(C)(C)c4ccccc4-5)c4ccccc4-c4ccccc4)cccc32)CCCC1] | 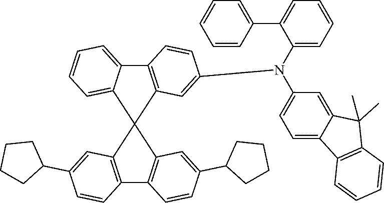 [SMILES: CC1(C)c2ccccc2-c2ccc(N(c3ccc4c(c3)C3(c5ccccc5-4)c4cc(C5CCCC5)ccc4-c4ccc(C5CCCC5)cc43)c3ccccc3-c3ccccc3)cc21] | 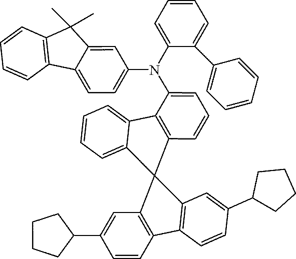 [SMILES: CC1(C)c2ccccc2-c2ccc(N(c3ccccc3-c3ccccc3)c3cccc4c3-c3ccccc3C43c4cc(C5CCCC5)ccc4-c4ccc(C5CCCC5)cc43)cc21] |
| CPD005 | CPD006 | CPD007 | CPD008 |
| 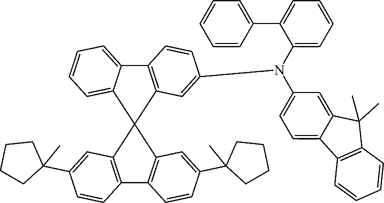 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCCC4)ccc2-3)CCCC1] | 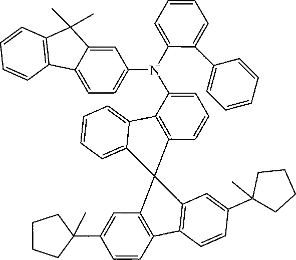 [SMILES: CC1(c2ccc3c(c2)C2(c4cc(C5(C)CCCC5)ccc4-3)c3ccccc3-c3c(N(c4ccc5c(c4)C(C)(C)c4ccccc4-5)c4ccccc4-c4ccccc4)cccc32)CCCC1] | 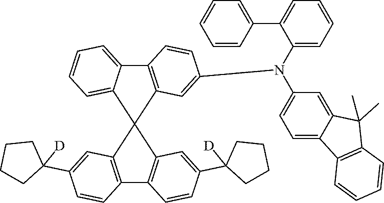 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCCC4)ccc2-3)CCCC1] | 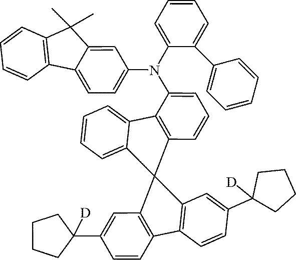 [SMILES: [2H]C1(c2ccc3c(c2)C2(c4cc(C5([2H])CCCC5)ccc4-3)c3ccccc3-c3c(N(c4ccc5c(c4)C(C)(C)c4ccccc4-5)c4ccccc4-c4ccccc4)cccc32)CCCC1] |
| CPD009 | CPD010 | CPD011 | CPD012 |

| | | | |
|---|---|---|---|
| 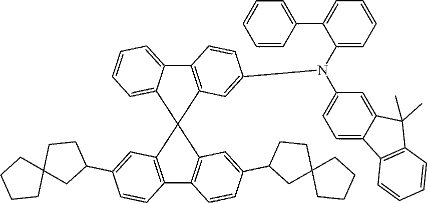 [SMILES: CC1(C)c2ccccc2-c2ccc(N(c3ccc4c(c3)C3(c5ccccc5-4)c4cc(C5CCC6(CCCC6)C5)ccc4-c4ccc(C5CCC6(CCCC6)C5)cc43)c3ccccc3-c3ccccc3)cc21] | 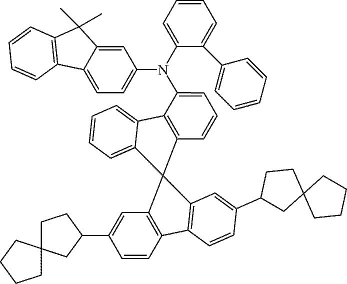 [SMILES: CC1(C)c2ccccc2-c2ccc(N(c3ccccc3-c3ccccc3)c3cccc4c3-c3ccccc3C43c4cc(C5CCC6(CCCC6)C5)ccc4-c4ccc(C5CCC6(CCCC6)C5)cc43)cc21] | 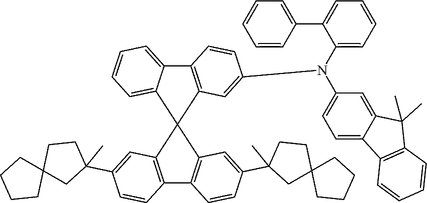 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC5(CCCC5)C4)ccc2-3)CCC2(CCCC2)C1] | 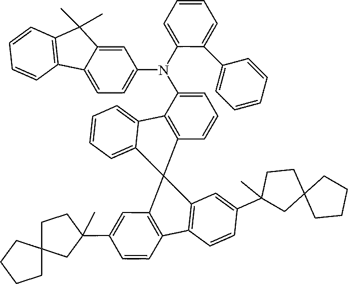 [SMILES: CC1(c2ccc3c(c2)C2(c4cc(C5(C)CCC6(CCCC6)C5)ccc4-3)c3ccccc3-c3c(N(c4ccc5c(c4)C(C)(C)c4ccccc4-5)c4ccccc4-c4ccccc4)cccc32)CCC2(CCCC2)C1] |
| CPD013 | CPD014 | CPD015 | CPD016 |
| 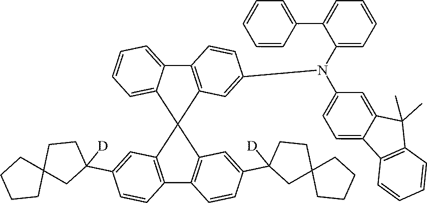 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC5(CCCC5)C4)ccc2-3)CCC2(CCCC2)C1] | 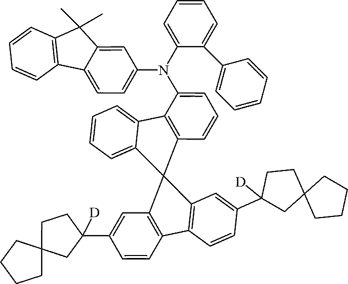 [SMILES: [2H]C1(c2ccc3c(c2)C2(c4cc(C5([2H])CCC6(CCCC6)C5)ccc4-3)c3ccccc3-c3c(N(c4ccc5c(c4)C(C)(C)c4ccccc4-5)c4ccccc4-c4ccccc4)cccc32)CCC2(CCCC2)C1] | 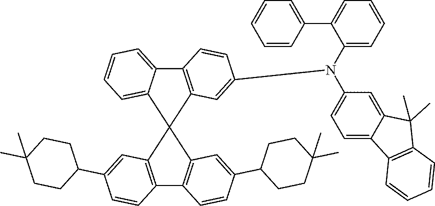 [SMILES: CC1(C)CCC(c2ccc3c(c2)C2(c4ccccc4-c4ccc(N(c5ccc6c(c5)C(C)(C)c5ccccc5-6)c5ccccc5-c5ccccc5)cc42)c2cc(C4CCC(C)(C)CC4)ccc2-3)CC1] | 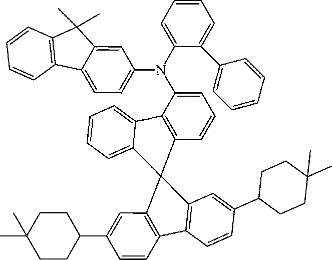 |
| CPD017 | CPD018 | CPD019 | CPD020 |
| 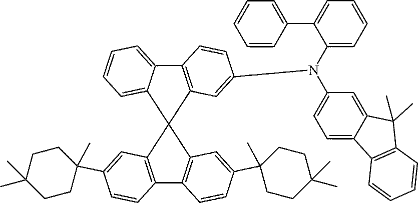 [SMILES: CC1(C)CCC(C)(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC(C)(C)CC4)ccc2-3)CC1] | 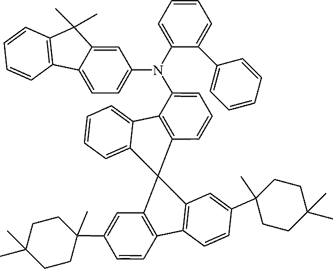 | 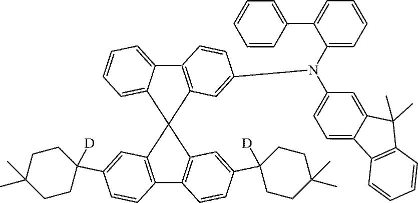 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC(C)(C)CC4)ccc2-3)CCC(C)(C)CC1] | 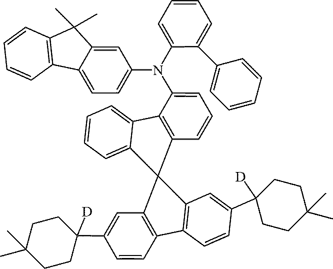 |
| CPD021 | CPD022 | CPD023 | CPD024 |
| 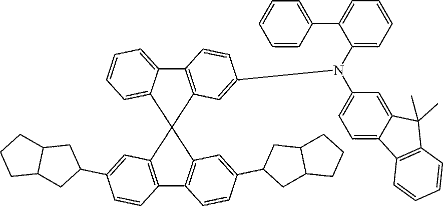 [SMILES: CC1(C)c2ccccc2-c2ccc(N(c3ccc4c(c3)C3(c5ccccc5-4)c4cc(C5CC6CCCC6C5)ccc4-c4ccc(C5CC6CCCC6C5)cc43)c3ccccc3-c3ccccc3)cc21] | 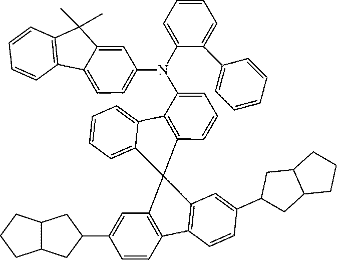 [SMILES: CC1(C)c2ccccc2-c2ccc(N(c3ccccc3-c3ccccc3)c3cccc4c3-c3ccccc3C43c4cc(C5CC6CCCC6C5)ccc4-c4ccc(C5CC6CCCC6C5)cc43)cc21] | 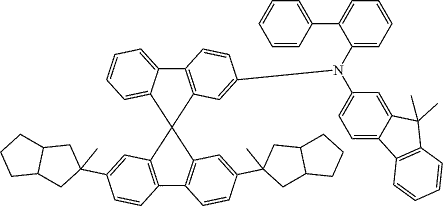 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CC5CCCC5C4)ccc2-3)CC2CCCC2C1] | 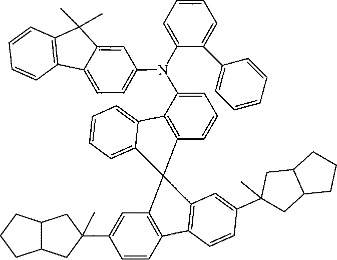 [SMILES: CC1(c2ccc3c(c2)C2(c4cc(C5(C)CC6CCCC6C5)ccc4-3)c3ccccc3-c3c(N(c4ccc5c(c4)C(C)(C)c4ccccc4-5)c4ccccc4-c4ccccc4)cccc32)CC2CCCC2C1] |
| CPD025 | CPD026 | CPD027 | CPD028 |
| 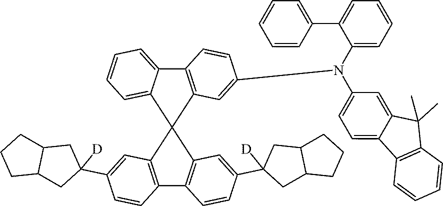 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CC5CCCC5C4)ccc2-3)CC2CCCC2C1] | 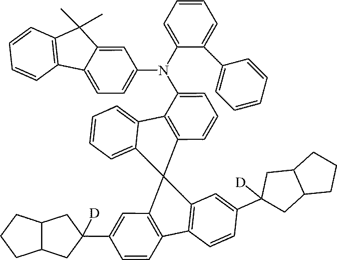 [SMILES: [2H]C1(c2ccc3c(c2)C2(c4cc(C5([2H])CC6CCCC6C5)ccc4-3)c3ccccc3-c3c(N(c4ccc5c(c4)C(C)(C)c4ccccc4-5)c4ccccc4-c4ccccc4)cccc32)CC2CCCC2C1] | 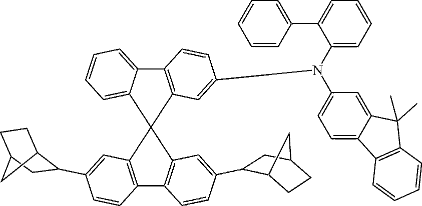 [SMILES: CC1(C)c2ccccc2-c2ccc(N(c3ccc4c(c3)C3(c5ccccc5-4)c4cc(C5CC6CCC5C6)ccc4-c4ccc(C5CC6CCC5C6)cc43)c3ccccc3-c3ccccc3)cc21] | 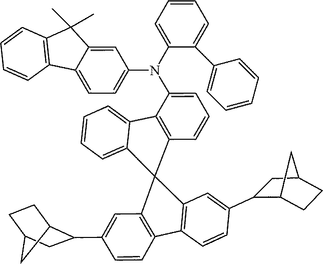 [SMILES: CC1(C)c2ccccc2-c2ccc(N(c3ccccc3-c3ccccc3)c3cccc4c3-c3ccccc3C43c4cc(C5CC6CCC5C6)ccc4-c4ccc(C5CC6CCC5C6)cc43)cc21] |
| CPD029 | CPD030 | CPD031 | CPD032 |

| | | | |
|---|---|---|---|
| 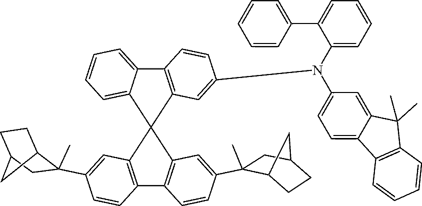 [SMILES: CC1(C)c2ccccc2-c2ccc(N(c3ccc4c(c3)C3(c5ccccc5-4)c4cc(C5(C)CC6CCC5C6)ccc4-c4ccc(C5(C)CC6CCC5C6)cc43)c3ccccc3-c3ccccc3)cc21] | 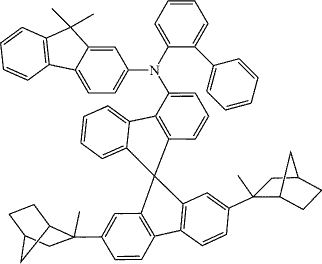 [SMILES: CC1(C)c2ccccc2-c2ccc(N(c3ccccc3-c3ccccc3)c3cccc4c3-c3ccccc3C43c4cc(C5(C)CC6CCC5C6)ccc4-c4ccc(C5(C)CC6CCC5C6)cc43)cc21] | 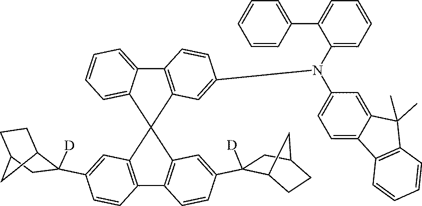 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CC5CCC4C5)ccc2-3)CC2CCC1C2] | 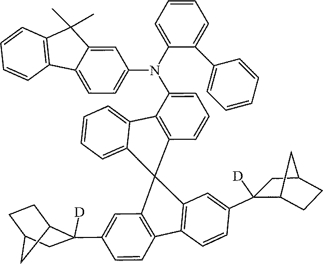 [SMILES: [2H]C1(c2ccc3c(c2)C2(c4cc(C5([2H])CC6CCC5C6)ccc4-3)c3ccccc3-c3c(N(c4ccc5c(c4)C(C)(C)c4ccccc4-5)c4ccccc4-c4ccccc4)cccc32)CC2CCC1C2] |
| CPD033 | CPD034 | CPD035 | CPD036 |
| 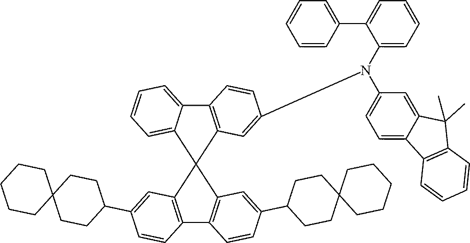 [SMILES: CC1(C)c2ccccc2-c2ccc(N(c3ccc4c(c3)C3(c5ccccc5-4)c4cc(C5CCC6(CCCCC6)CC5)ccc4-c4ccc(C5CCC6(CCCCC6)CC5)cc43)c3ccccc3-c3ccccc3)cc21] | 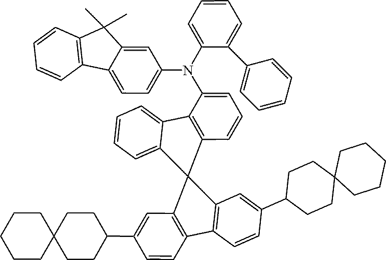 [SMILES: CC1(C)c2ccccc2-c2ccc(N(c3ccccc3-c3ccccc3)c3cccc4c3-c3ccccc3C43c4cc(C5CCC6(CCCCC6)CC5)ccc4-c4ccc(C5CCC6(CCCCC6)CC5)cc43)cc21] | 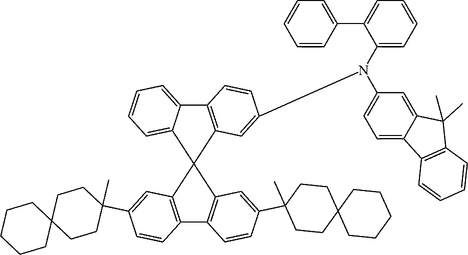 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC5(CCCCC5)CC4)ccc2-3)CCC2(CCCCC2)CC1] | 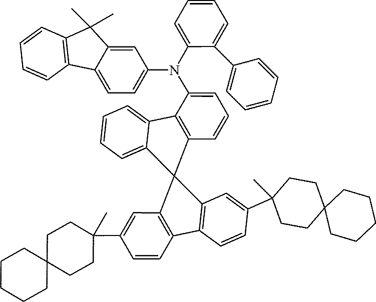 [SMILES: CC1(c2ccc3c(c2)C2(c4cc(C5(C)CCC6(CCCCC6)CC5)ccc4-3)c3ccccc3-c3c(N(c4ccc5c(c4)C(C)(C)c4ccccc4-5)c4ccccc4-c4ccccc4)cccc32)CCC2(CCCCC2)CC1] |
| CPD037 | CPD038 | CPD039 | CPD040 |
| 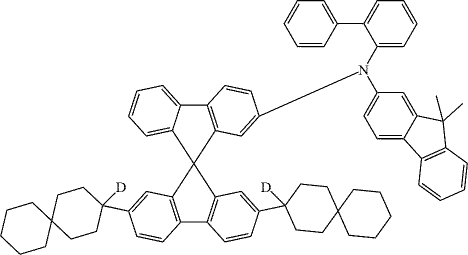 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC5(CCCCC5)CC4)ccc2-3)CCC2(CCCCC2)CC1] | 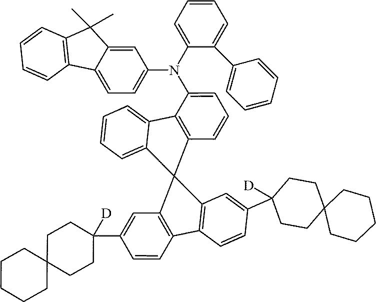 [SMILES: [2H]C1(c2ccc3c(c2)C2(c4cc(C5([2H])CCC6(CCCCC6)CC5)ccc4-3)c3ccccc3-c3c(N(c4ccc5c(c4)C(C)(C)c4ccccc4-5)c4ccccc4-c4ccccc4)cccc32)CCC2(CCCCC2)CC1] | 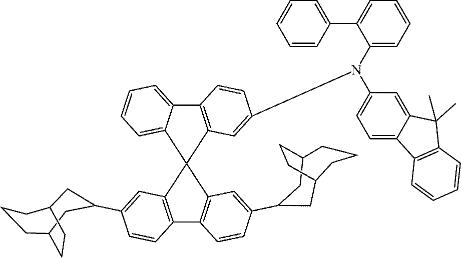 [SMILES: CC1(C)c2ccccc2-c2ccc(N(c3ccc4c(c3)C3(c5ccccc5-4)c4cc(C5CC6CCCC(CC6)C5)ccc4-c4ccc(C5CC6CCCC(CC6)C5)cc43)c3ccccc3-c3ccccc3)cc21] | 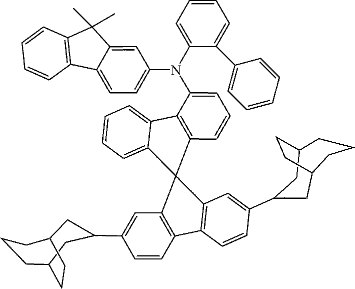 [SMILES: CC1(C)c2ccccc2-c2ccc(N(c3ccccc3-c3ccccc3)c3cccc4c3-c3ccccc3C43c4cc(C5CC6CCCC(CC6)C5)ccc4-c4ccc(C5CC6CCCC(CC6)C5)cc43)cc21] |
| CPD041 | CPD042 | CPD043 | CPD044 |
| 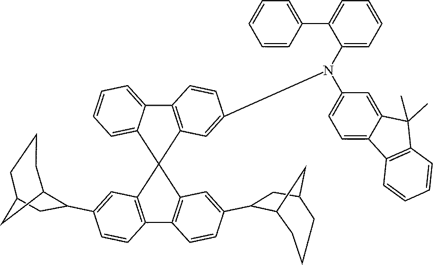 [SMILES: CC1(C)c2ccccc2-c2ccc(N(c3ccc4c(c3)C3(c5ccccc5-4)c4cc(C5CC6CCCC5C6)ccc4-c4ccc(C5CC6CCCC5C6)cc43)c3ccccc3-c3ccccc3)cc21] | 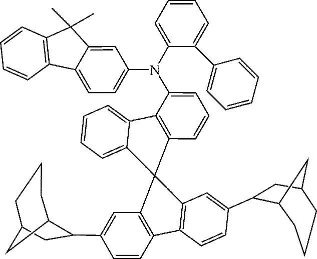 [SMILES: CC1(C)c2ccccc2-c2ccc(N(c3ccccc3-c3ccccc3)c3cccc4c3-c3ccccc3C43c4cc(C5CC6CCCC5C6)ccc4-c4ccc(C5CC6CCCC5C6)cc43)cc21] | 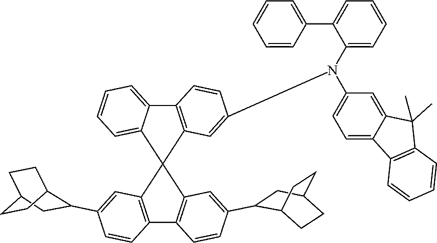 [SMILES: CC1(C)c2ccccc2-c2ccc(N(c3ccc4c(c3)C3(c5ccccc5-4)c4cc(C5CC6CCC5CC6)ccc4-c4ccc(C5CC6CCC5CC6)cc43)c3ccccc3-c3ccccc3)cc21] | 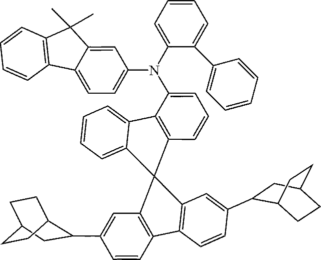 [SMILES: CC1(C)c2ccccc2-c2ccc(N(c3ccccc3-c3ccccc3)c3cccc4c3-c3ccccc3C43c4cc(C5CC6CCC5CC6)ccc4-c4ccc(C5CC6CCC5CC6)cc43)cc21] |
| CPD045 | CPD046 | CPD047 | CPD048 |
| 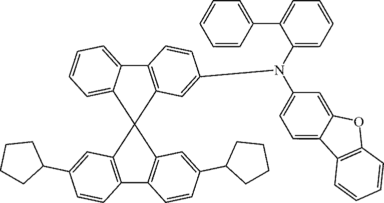 [SMILES: c1ccc(-c2ccccc2N(c2ccc3c(c2)C2(c4ccccc4-3)c3cc(C4CCCC4)ccc3-c3ccc(C4CCCC4)cc32)c2ccc3c(c2)oc2ccccc23)cc1] | 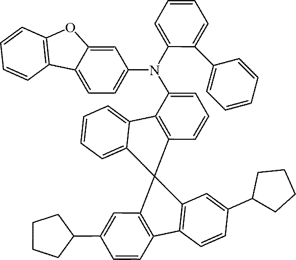 [SMILES: c1ccc(-c2ccccc2N(c2ccc3c(c2)oc2ccccc23)c2cccc3c2-c2ccccc2C32c3cc(C4CCCC4)ccc3-c3ccc(C4CCCC4)cc32)cc1] | 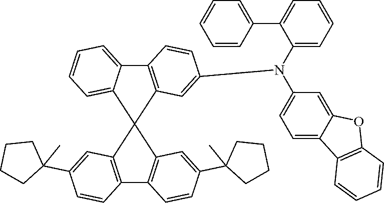 [SMILES: CC1(c2ccc3c(c2)C2(c4ccccc4-c4ccc(N(c5ccc6c(c5)oc5ccccc56)c5ccccc5-c5ccccc5)cc42)c2cc(C4(C)CCCC4)ccc2-3)CCCC1] | 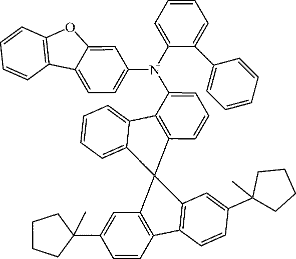 [SMILES: CC1(c2ccc3c(c2)C2(c4cc(C5(C)CCCC5)ccc4-3)c3ccccc3-c3c(N(c4ccc5c(c4)oc4ccccc45)c4ccccc4-c4ccccc4)cccc32)CCCC1] |
| CPD049 | CPD050 | CPD051 | CPD052 |

| | | | |
|---|---|---|---|
| 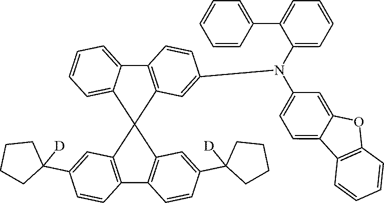 [SMILES: [2H]C1(c2ccc3c(c2)C2(c4ccccc4-c4ccc(N(c5ccc6c(c5)oc5ccccc56)c5ccccc5-c5ccccc5)cc42)c2cc(C4([2H])CCCC4)ccc2-3)CCCC1] | 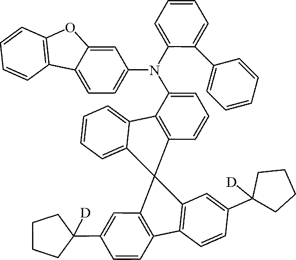 [SMILES: [2H]C1(c2ccc3c(c2)C2(c4cc(C5([2H])CCCC5)ccc4-3)c3ccccc3-c3c(N(c4ccc5c(c4)oc4ccccc45)c4ccccc4-c4ccccc4)cccc32)CCCC1] | 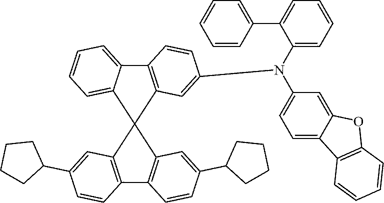 [SMILES: c1ccc(-c2ccccc2N(c2ccc3c(c2)C2(c4ccccc4-3)c3cc(C4CCCC4)ccc3-c3ccc(C4CCCC4)cc32)c2ccc3c(c2)oc2ccccc23)cc1] | 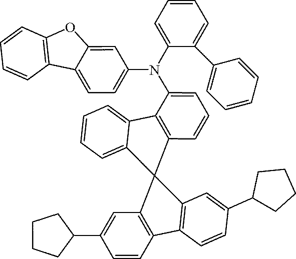 [SMILES: c1ccc(-c2ccccc2N(c2ccc3c(c2)oc2ccccc23)c2cccc3c2-c2ccccc2C32c3cc(C4CCCC4)ccc3-c3ccc(C4CCCC4)cc32)cc1] |
| CPD053 | CPD054 | CPD055 | CPD056 |
| 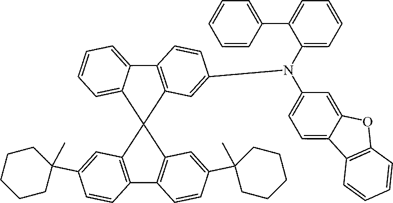 [SMILES: CC1(c2ccc3c(c2)C2(c4ccccc4-c4ccc(N(c5ccc6c(c5)oc5ccccc56)c5ccccc5-c5ccccc5)cc42)c2cc(C4(C)CCCCC4)ccc2-3)CCCCC1] | 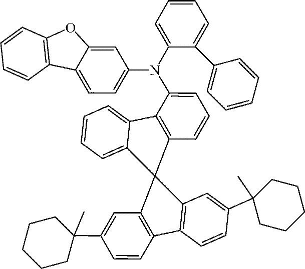 [SMILES: CC1(c2ccc3c(c2)C2(c4cc(C5(C)CCCCC5)ccc4-3)c3ccccc3-c3c(N(c4ccc5c(c4)oc4ccccc45)c4ccccc4-c4ccccc4)cccc32)CCCCC1] | 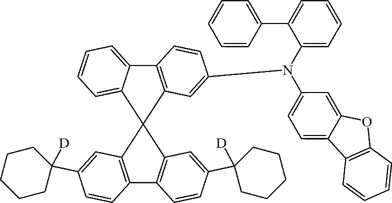 [SMILES: [2H]C1(c2ccc3c(c2)C2(c4ccccc4-c4ccc(N(c5ccc6c(c5)oc5ccccc56)c5ccccc5-c5ccccc5)cc42)c2cc(C4([2H])CCCCC4)ccc2-3)CCCCC1] | 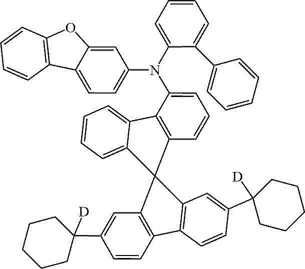 [SMILES: [2H]C1(c2ccc3c(c2)C2(c4cc(C5([2H])CCCCC5)ccc4-3)c3ccccc3-c3c(N(c4ccc5c(c4)oc4ccccc45)c4ccccc4-c4ccccc4)cccc32)CCCCC1] |
| CPD057 | CPD058 | CPD059 | CPD060 |
| 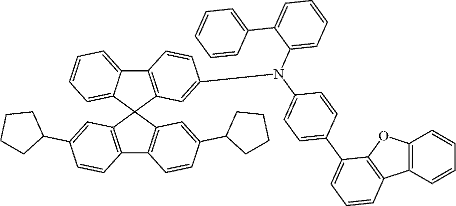 [SMILES: c1ccc(-c2ccccc2N(c2ccc(-c3cccc4c3oc3ccccc34)cc2)c2ccc3c(c2)C2(c4ccccc4-3)c3cc(C4CCCC4)ccc3-c3ccc(C4CCCC4)cc32)cc1] | 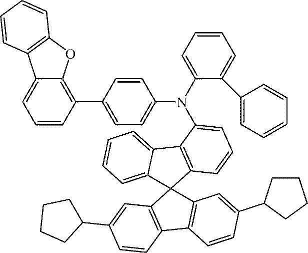 [SMILES: c1ccc(-c2ccccc2N(c2ccc(-c3cccc4c3oc3ccccc34)cc2)c2cccc3c2-c2ccccc2C32c3cc(C4CCCC4)ccc3-c3ccc(C4CCCC4)cc32)cc1] | 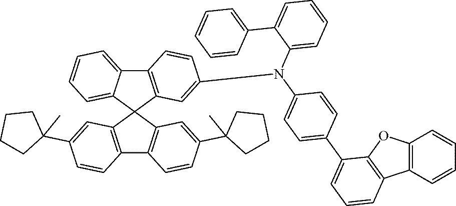 [SMILES: CC1(c2ccc3c(c2)C2(c4ccccc4-c4ccc(N(c5ccc(-c6cccc7c6oc6ccccc67)cc5)c5ccccc5-c5ccccc5)cc42)c2cc(C4(C)CCCC4)ccc2-3)CCCC1] | 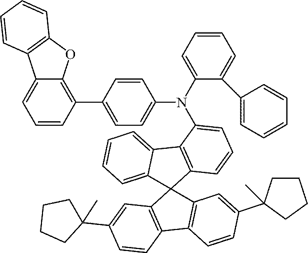 [SMILES: CC1(c2ccc3c(c2)C2(c4cc(C5(C)CCCC5)ccc4-3)c3ccccc3-c3c(N(c4ccc(-c5cccc6c5oc5ccccc56)cc4)c4ccccc4-c4ccccc4)cccc32)CCCC1] |
| CPD061 | CPD062 | CPD063 | CPD064 |
| 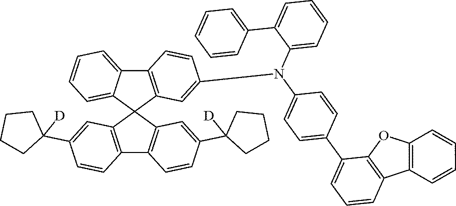 [SMILES: [2H]C1(c2ccc3c(c2)C2(c4ccccc4-c4ccc(N(c5ccc(-c6cccc7c6oc6ccccc67)cc5)c5ccccc5-c5ccccc5)cc42)c2cc(C4([2H])CCCC4)ccc2-3)CCCC1] | 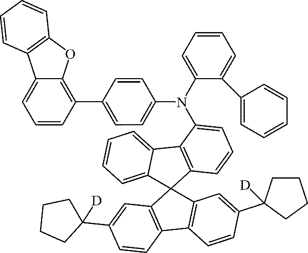 [SMILES: [2H]C1(c2ccc3c(c2)C2(c4cc(C5([2H])CCCC5)ccc4-3)c3ccccc3-c3c(N(c4ccc(-c5cccc6c5oc5ccccc56)cc4)c4ccccc4-c4ccccc4)cccc32)CCCC1] | 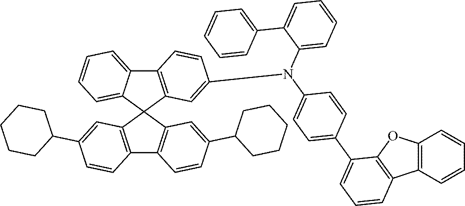 [SMILES: c1ccc(-c2ccccc2N(c2ccc(-c3cccc4c3oc3ccccc34)cc2)c2ccc3c(c2)C2(c4ccccc4-3)c3cc(C4CCCCC4)ccc3-c3ccc(C4CCCCC4)cc32)cc1] | 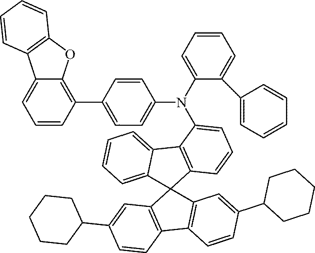 [SMILES: c1ccc(-c2ccccc2N(c2ccc(-c3cccc4c3oc3ccccc34)cc2)c2cccc3c2-c2ccccc2C32c3cc(C4CCCCC4)ccc3-c3ccc(C4CCCCC4)cc32)cc1] |
| CPD065 | CPD066 | CPD067 | CPD068 |
| 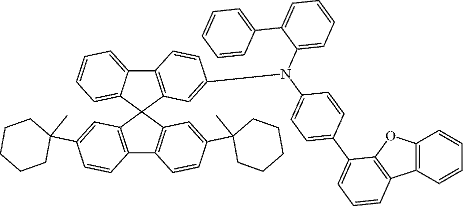 [SMILES: CC1(c2ccc3c(c2)C2(c4ccccc4-c4ccc(N(c5ccc(-c6cccc7c6oc6ccccc67)cc5)c5ccccc5-c5ccccc5)cc42)c2cc(C4(C)CCCCC4)ccc2-3)CCCCC1] | 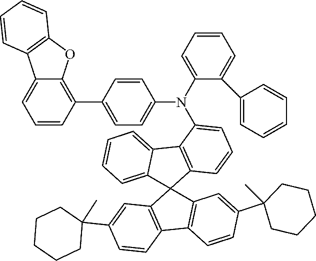 [SMILES: CC1(c2ccc3c(c2)C2(c4cc(C5(C)CCCCC5)ccc4-3)c3ccccc3-c3c(N(c4ccc(-c5cccc6c5oc5ccccc56)cc4)c4ccccc4-c4ccccc4)cccc32)CCCCC1] | 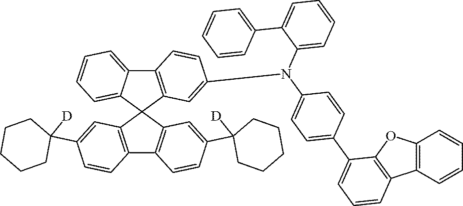 [SMILES: [2H]C1(c2ccc3c(c2)C2(c4ccccc4-c4ccc(N(c5ccc(-c6cccc7c6oc6ccccc67)cc5)c5ccccc5-c5ccccc5)cc42)c2cc(C4([2H])CCCCC4)ccc2-3)CCCCC1] | 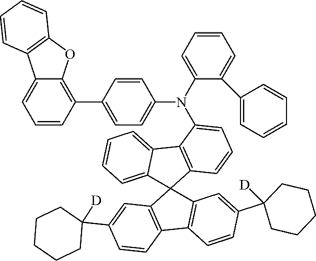 [SMILES: [2H]C1(c2ccc3c(c2)C2(c4cc(C5([2H])CCCCC5)ccc4-3)c3ccccc3-c3c(N(c4ccc(-c5cccc6c5oc5ccccc56)cc4)c4ccccc4-c4ccccc4)cccc32)CCCCC1] |
| CPD069 | CPD070 | CPD071 | CPD072 |

| | | | |
|---|---|---|---|
| 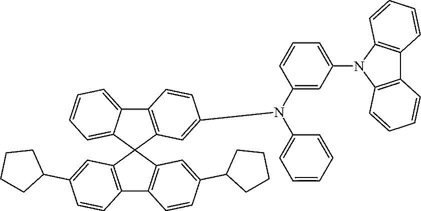 [SMILES: c1ccc(N(c2cccc(-n3c4ccccc4c4ccccc43)c2)c2ccc3c(c2)C2(c4ccccc4-3)c3cc(C4CCCC4)ccc3-c3ccc(C4CCCC4)cc32)cc1] | 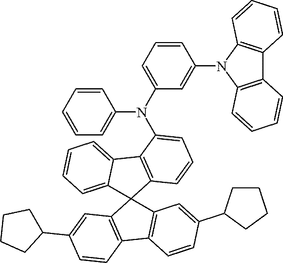 [SMILES: c1ccc(N(c2cccc(-n3c4ccccc4c4ccccc43)c2)c2cccc3c2-c2ccccc2C32c3cc(C4CCCC4)ccc3-c3ccc(C4CCCC4)cc32)cc1] | 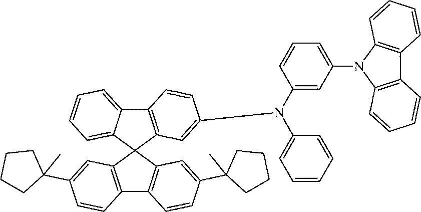 [SMILES: CC1(c2ccc3c(c2)C2(c4ccccc4-c4ccc(N(c5ccccc5)c5cccc(-n6c7ccccc7c7ccccc76)c5)cc42)c2cc(C4(C)CCCC4)ccc2-3)CCCC1] | 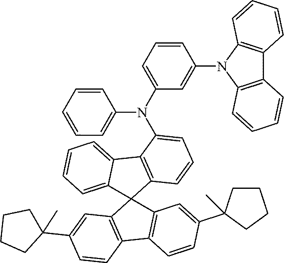 [SMILES: CC1(c2ccc3c(c2)C2(c4cc(C5(C)CCCC5)ccc4-3)c3ccccc3-c3c(N(c4ccccc4)c4cccc(-n5c6ccccc6c6ccccc65)c4)cccc32)CCCC1] |
| CPD073 | CPD074 | CPD075 | CPD076 |
| 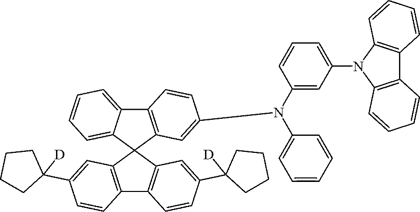 [SMILES: [2H]C1(c2ccc3c(c2)C2(c4ccccc4-c4ccc(N(c5ccccc5)c5cccc(-n6c7ccccc7c7ccccc76)c5)cc42)c2cc(C4([2H])CCCC4)ccc2-3)CCCC1] | 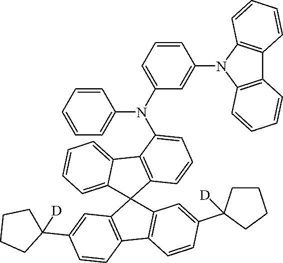 [SMILES: [2H]C1(c2ccc3c(c2)C2(c4cc(C5([2H])CCCC5)ccc4-3)c3ccccc3-c3c(N(c4ccccc4)c4cccc(-n5c6ccccc6c6ccccc65)c4)cccc32)CCCC1] | 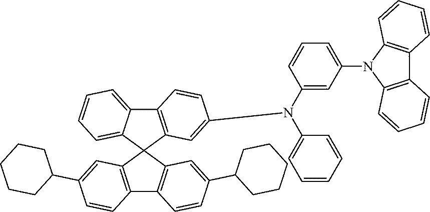 [SMILES: c1ccc(N(c2cccc(-n3c4ccccc4c4ccccc43)c2)c2ccc3c(c2)C2(c4ccccc4-3)c3cc(C4CCCCC4)ccc3-c3ccc(C4CCCCC4)cc32)cc1] | 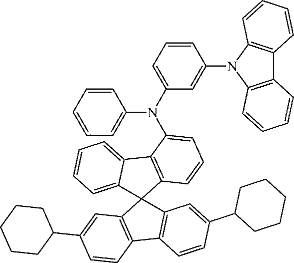 [SMILES: c1ccc(N(c2cccc(-n3c4ccccc4c4ccccc43)c2)c2cccc3c2-c2ccccc2C32c3cc(C4CCCCC4)ccc3-c3ccc(C4CCCCC4)cc32)cc1] |
| CPD077 | CPD078 | CPD079 | CPD080 |
| 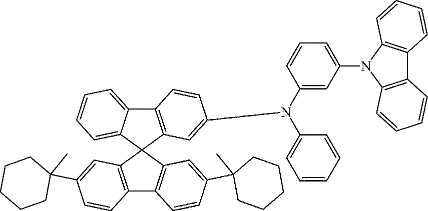 [SMILES: CC1(c2ccc3c(c2)C2(c4ccccc4-c4ccc(N(c5ccccc5)c5cccc(-n6c7ccccc7c7ccccc76)c5)cc42)c2cc(C4(C)CCCCC4)ccc2-3)CCCCC1] | 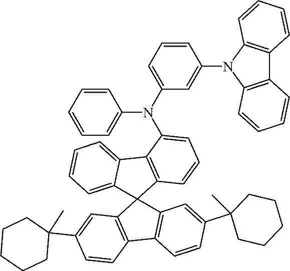 [SMILES: CC1(c2ccc3c(c2)C2(c4cc(C5(C)CCCCC5)ccc4-3)c3ccccc3-c3c(N(c4ccccc4)c4cccc(-n5c6ccccc6c6ccccc65)c4)cccc32)CCCCC1] | 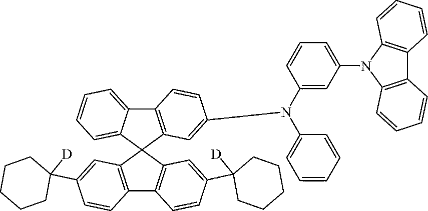 [SMILES: [2H]C1(c2ccc3c(c2)C2(c4ccccc4-c4ccc(N(c5ccccc5)c5cccc(-n6c7ccccc7c7ccccc76)c5)cc42)c2cc(C4([2H])CCCCC4)ccc2-3)CCCCC1] | 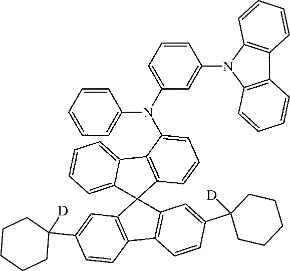 [SMILES: [2H]C1(c2ccc3c(c2)C2(c4cc(C5([2H])CCCCC5)ccc4-3)c3ccccc3-c3c(N(c4ccccc4)c4cccc(-n5c6ccccc6c6ccccc65)c4)cccc32)CCCCC1] |
| CPD081 | CPD082 | CPD083 | CPD084 |
| 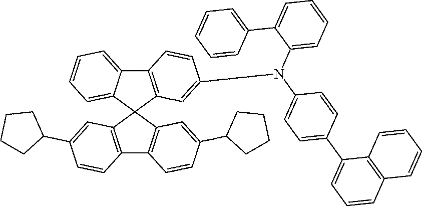 [SMILES: c1ccc(-c2ccccc2N(c2ccc(-c3cccc4ccccc34)cc2)c2ccc3c(c2)C2(c4ccccc4-3)c3cc(C4CCCC4)ccc3-c3ccc(C4CCCC4)cc32)cc1] | 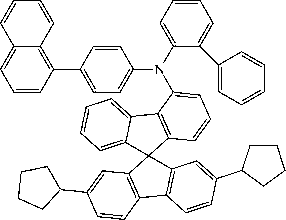 [SMILES: c1ccc(-c2ccccc2N(c2ccc(-c3cccc4ccccc34)cc2)c2cccc3c2-c2ccccc2C32c3cc(C4CCCC4)ccc3-c3ccc(C4CCCC4)cc32)cc1] | 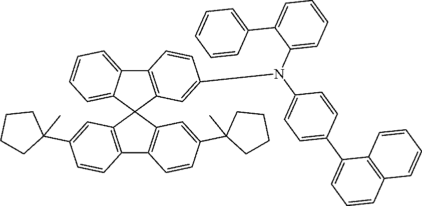 [SMILES: CC1(c2ccc3c(c2)C2(c4ccccc4-c4ccc(N(c5ccc(-c6cccc7ccccc67)cc5)c5ccccc5-c5ccccc5)cc42)c2cc(C4(C)CCCC4)ccc2-3)CCCC1] | 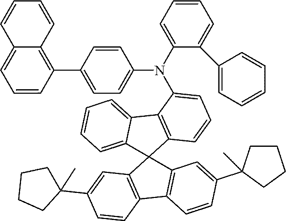 [SMILES: CC1(c2ccc3c(c2)C2(c4cc(C5(C)CCCC5)ccc4-3)c3ccccc3-c3c(N(c4ccc(-c5cccc6ccccc56)cc4)c4ccccc4-c4ccccc4)cccc32)CCCC1] |
| CPD085 | CPD086 | CPD087 | CPD088 |
| 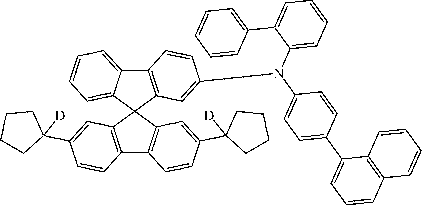 [SMILES: [2H]C1(c2ccc3c(c2)C2(c4ccccc4-c4ccc(N(c5ccc(-c6cccc7ccccc67)cc5)c5ccccc5-c5ccccc5)cc42)c2cc(C4([2H])CCCC4)ccc2-3)CCCC1] | 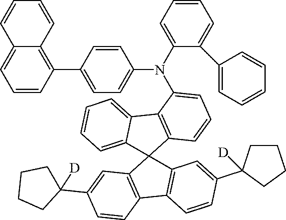 [SMILES: [2H]C1(c2ccc3c(c2)C2(c4cc(C5([2H])CCCC5)ccc4-3)c3ccccc3-c3c(N(c4ccc(-c5cccc6ccccc56)cc4)c4ccccc4-c4ccccc4)cccc32)CCCC1] | 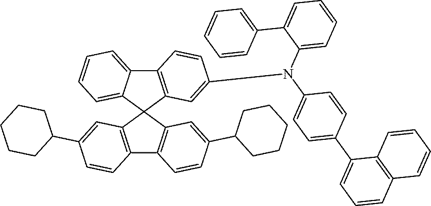 [SMILES: c1ccc(-c2ccccc2N(c2ccc(-c3cccc4ccccc34)cc2)c2ccc3c(c2)C2(c4ccccc4-3)c3cc(C4CCCCC4)ccc3-c3ccc(C4CCCCC4)cc32)cc1] | 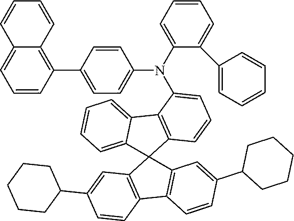 [SMILES: c1ccc(-c2ccccc2N(c2ccc(-c3cccc4ccccc34)cc2)c2cccc3c2-c2ccccc2C32c3cc(C4CCCCC4)ccc3-c3ccc(C4CCCCC4)cc32)cc1] |

| | | | |
|---|---|---|---|
| CDP089 | CPD090 | CPD091 | CPD092 |
| 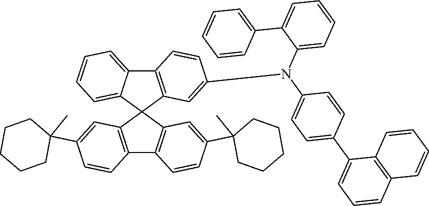 | 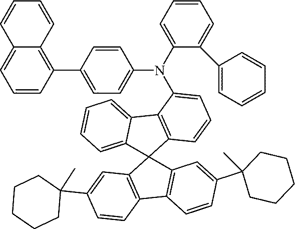 | 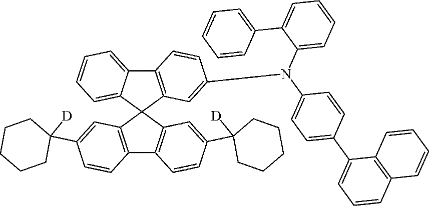 | 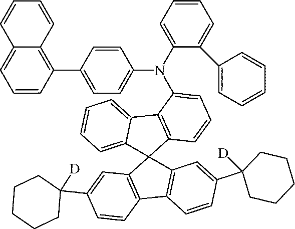 |
| CPD093 | CPD094 | CPD095 | CPD096 |
| 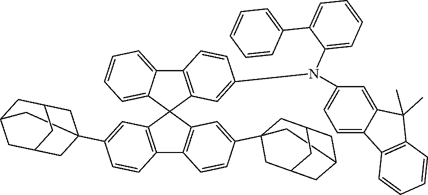 | 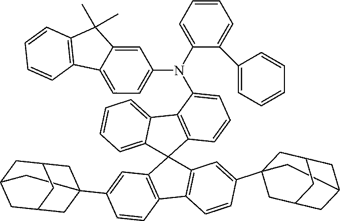 | 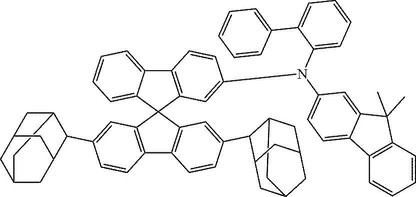 | 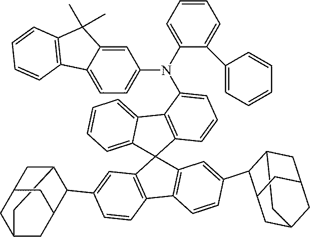 |
| CPD097 | CPD098 | CPD099 | CPD100 |
| 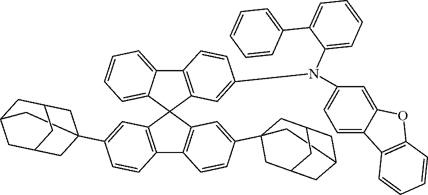 | 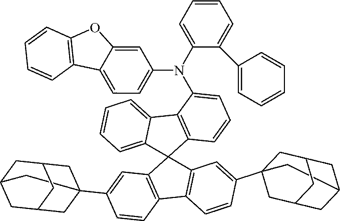 | 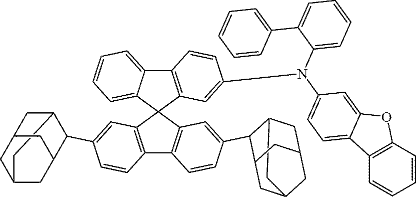 | 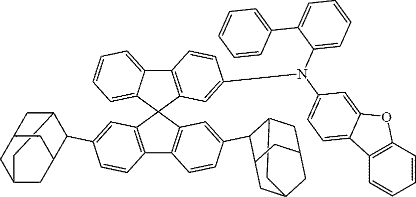 |
| CPD101 | CPD102 | CPD103 | CPD104 |
| 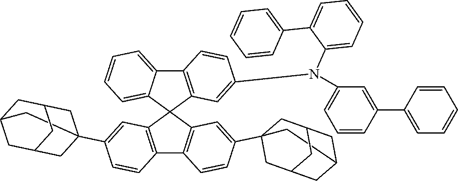 | 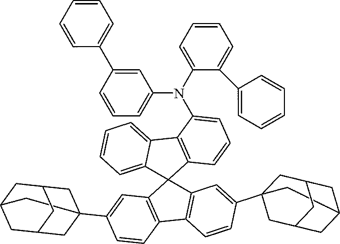 | 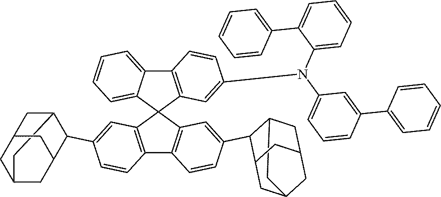 | 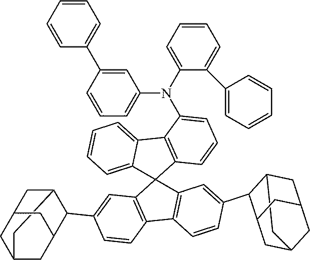 |
| CPD105 | CPD106 | CPD107 | CPD108 |
| 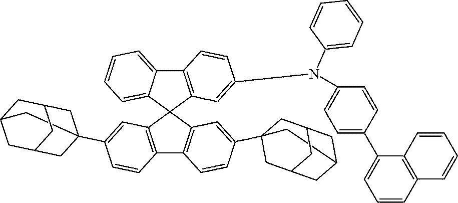 | 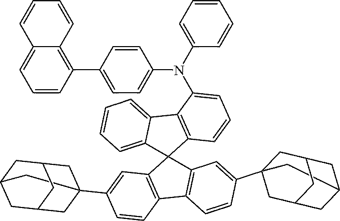 | 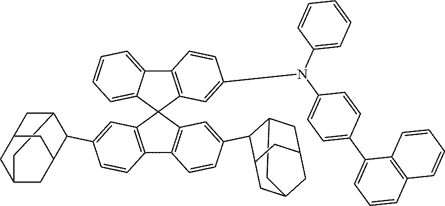 | 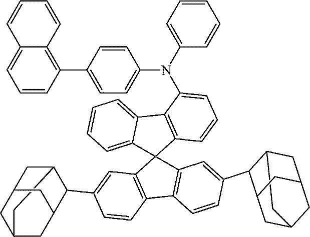 |
| CPD109 | CPD110 | CPD111 | CPD112 |

| | | | |
|---|---|---|---|
| 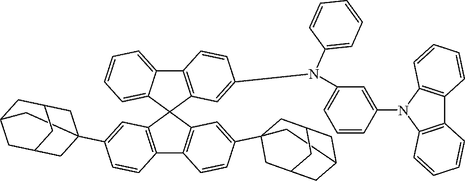 | 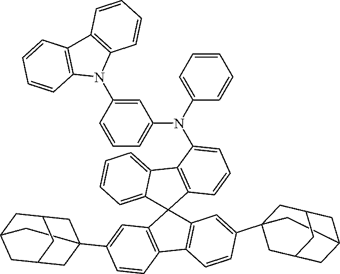 | 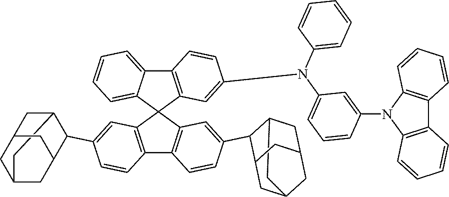 | 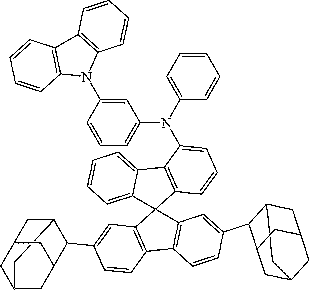 |
| CPD113 | CPD114 | CPD115 | CPD116 |
| 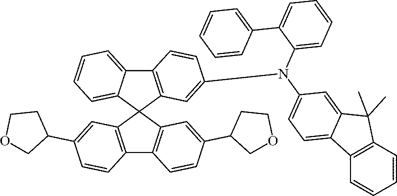 | 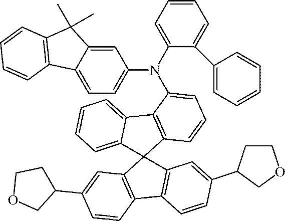 | 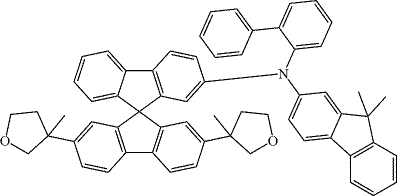 | 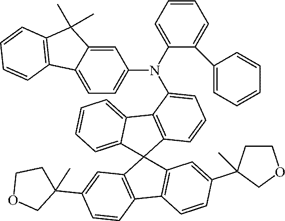 |
| CPD117 | CPD118 | CPD119 | CPD120 |
| 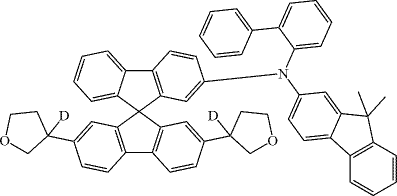 | 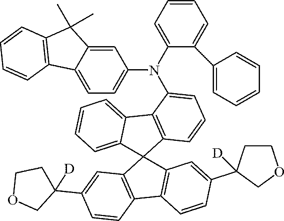 | 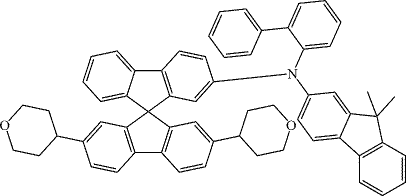 | 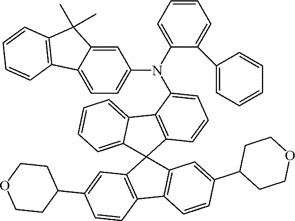 |
| 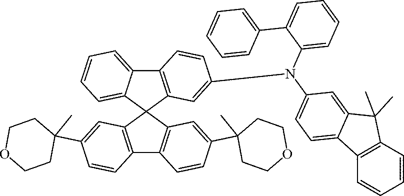 | 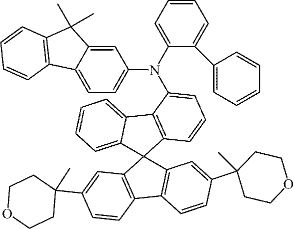 | 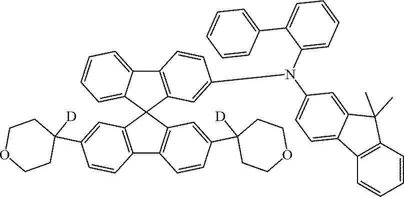 | 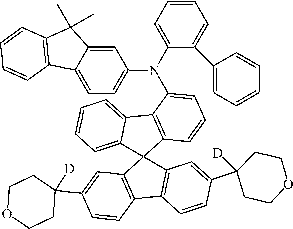 |
| CPD121 | CPD122 | CPD123 | CPD124 |

Another objective of the present disclosure is to provide application of the spiro compound in an organic electroluminescent device.

Another objective of the present disclosure is to provide use of the spiro compound as a hole injection layer and/or a hole transport layer of an organic electroluminescent device.

The material of the present disclosure has advantages such as high optical and electrical stability, low sublimation temperature, low drive current, low lateral mobility of carriers, high luminous efficiency, and long service life of a device, and can be used in an organic electroluminescent device. In particular, the compound has the possibility of being applied in the AMOLED industry as a hole injection or transport material.

### BRIEF DESCRIPTION OF DRAWINGS

FIG. 1 is a diagram showing the ¹HNMR spectrum of a compound CPD001.

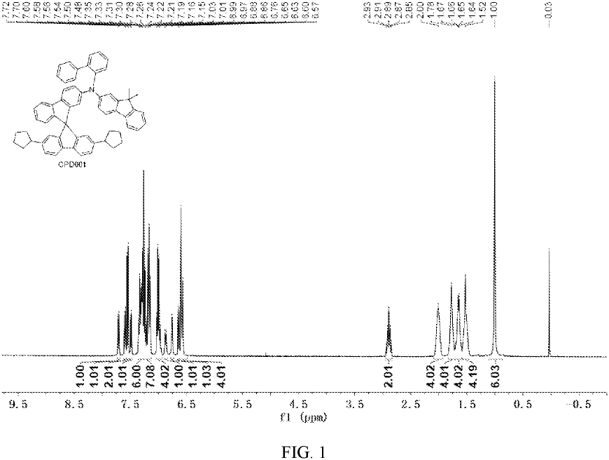

### DETAILED DESCRIPTION OF EMBODIMENTS

The present disclosure is further described in detail below in conjunction with embodiments.

A compound, namely a spiro compound, of the present disclosure has a structure as shown in a formula (1),

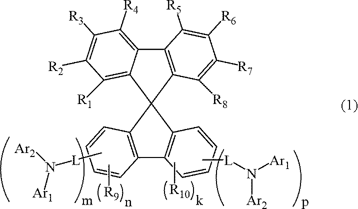

where R₁-R₁₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀ heteroalkyl, substituted or unsubstituted C₃-C₂₀ cycloalkyl, substituted or unsubstituted C₃-C₂₀ heterocyclic alkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀ alkyl silyl, substituted or unsubstituted tri-C₆-C₁₂ aryl silyl, substituted or unsubstituted di-C₁-C₁₀ alkyl mono-C₆-C₃₀ aryl silyl, and substituted or unsubstituted mono-C₁-C₁₀ alkyl di-C₆-C₃₀ aryl silyl, or two adjacent groups of R₁-R₈ and R_{y}-R₁₀ may be connected to each other to form an aliphatic ring or an aromatic ring structure; the “substituted” refers to substitution with deuterium, F, Cl, Br, C₁-C₆ alkyl, C₃-C₆ cycloalkyl, amino substituted with C₁-C₆ alkyl, cyano, isonitrile, or phosphino, and the substitution number ranges from a single substitution number to a maximum substitution number;
L is independently selected from a single bond, substituted or unsubstituted C₆-C₃₀arylene, or substituted or unsubstituted C₂-C₃₀ heteroarylene;
Ar₁ and Ar₂ are independently selected from substituted or unsubstituted C₆-C₃₀ aryl, or substituted or unsubstituted C₂-C₃₀ heteroaryl;
m, n, h, and p are independently selected from 0 or an integer of 1-4, m+n=4, and p+k=4;
the heteroalkyl and the heteroaryl at least contain one O, N, or S heteroatom; and
at least two groups of the R₁-R₈ are substituted or unsubstituted C₃-C₂₀ cycloalkyl, or substituted or unsubstituted C₃-C₂₀ heterocyclic alkyl.

Examples of various groups of the compound as shown in the formula (1) are described below.

It should be noted that in the specification, “Cₐ-C_{b}” in the term “substituted or unsubstituted Cₐ-C_{b} X group” refers to the number of carbons when the X group is unsubstituted, excluding the number of carbons of a substituent when the X group is substituted.

As a linear or branched alkyl, the C₁-C₁₀ alkyl specifically includes methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, and n-decyl and isomers thereof, preferably methyl, ethyl, propyl, isopropyl, n-butyl, isobutyl, sec-butyl, and tert-butyl, and more preferably propyl, isopropyl, isobutyl, sec-butyl, and tert-butyl.

The C₃-C₂₀ cycloalkyl may include cyclopropyl, cyclobutyl, cyclopentyl, cyclohexyl, 1-adamantyl, 2-adamantyl, 1-norbornyl, and 2-norbornyl, preferably cyclopentyl and cyclohexyl.

The C₂-C₁₀ alkenyl may include vinyl, propenyl, allyl, 1-butadienyl, 2-butadienyl, 1-hexatrienyl, 2-hexatrienyl, and 3-hexatrienyl, preferably propeny and allyl.

As a linear or branched alkyl or cycloalkyl consisting of atoms other than carbon and hydrogen, the C₁-C₁₀ heteroalkyl may include mercaptomethyl methyl, methoxymethyl, ethoxymethyl, tert-butoxyl methyl, N,N-dimethyl methyl, epoxy butyl, epoxy pentyl, and epoxy hexyl, preferably methoxymethyl and epoxy pentyl.

Specific examples of the aryl include phenyl, naphthyl, anthracyl, phenanthryl, tetracenyl, pyrenyl, chrysenyl, benzo[c]phenanthryl, benzo[g]chrysenyl, fluorenyl, benzofluorenyl, dibenzofluorenyl, biphenyl, triphenyl, tetraphenyl, and fluoranthracyl, preferably phenyl and naphthyl.

Specific examples of the heteroaryl may include pyrrolyl, pyrazinyl, pyridyl, pyrimidinyl, triazinyl, indolyl, isoindolyl, imidazolyl, furyl, benzofuryl, isobenzofuryl, dibenzofuryl, dibenzothienyl, azodibenzofuryl, azodibenzothienyl, diazodibenzofuryl, diazodibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furzanyl, thienyl, benzothienyl, dihydroacridinyl, azocarbazolyl, diazocarbazolyl, and quinazolinyl, preferably pyridyl, pyrimidinyl, triazinyl, dibenzofuryl, dibenzothienyl, azodibenzofuryl, azodibenzothienyl, diazodibenzofuryl, diazodibenzothienyl, carbazolyl, azocarbazolyl, and diazocarbazolyl.

The following embodiments are merely described to facilitate the understanding of the technical disclosure, and should not be considered as specific limitations of the present disclosure.

All raw materials, solvents and the like involved in the synthesis of compounds in the present disclosure are purchased from Alfa, Acros, and other suppliers known to persons skilled in the art.

### Synthesis of a Compound CPD001

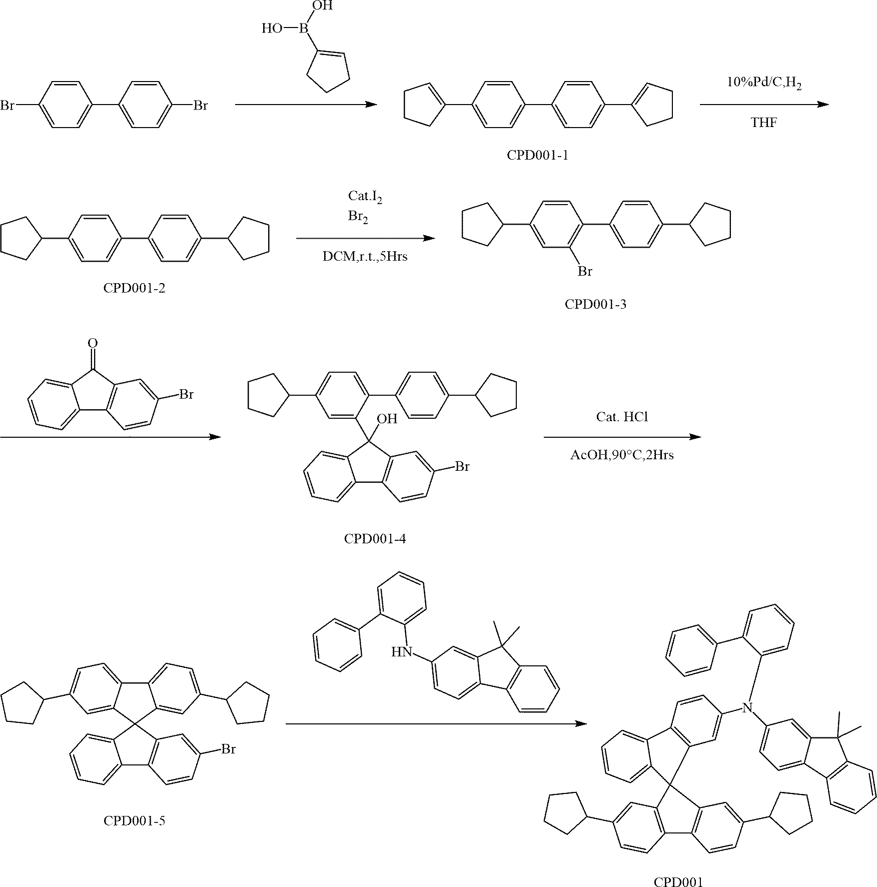

### Synthesis of a Compound CPD001-1

A compound 4,4′-dibromobiphenyl (18.00 g, 57.69 mmol), cyclopentene-1-ylboric acid (16.14 g, 144.23 mmol), bis(4-dimethylaminophenyldi-tert-butylphosphine)palladium dichloride (0.41 g, 0.57 mmol), potassium carbonate (31.89 g, 230.77 mmol), tetrahydrofuran (270 ml), and deionized water (90 ml) were added to a 1,000 ml three-mouth round-bottomed flask, subjected to nitrogen replacement for four times, and heated to 60° C. for a reaction overnight. According to monitoring by TLC (with n-hexane as a developing agent), the raw material 4,4′-dibromobiphenyl was completely consumed.

The system was cooled to room temperature, deionized water (100 ml) and methanol (200 ml) were added and stirred at room temperature for 2 hours, suction filtration was conducted, and a solid was washed with methanol and water and then dried overnight at 90° C. to obtain a gray solid, namely a compound CPD001-1 (16.18 g, purity: 99.99%, and yield: 97.94%). The mass spectrum was 287.26 (M+H).

### Synthesis of a Compound CPD001-2

The compound CPD001-1 (28.23 g, 98.56 mmol) and tetrahydrofuran (1,400 ml) were added to a 2,000 ml four-mouth round-bottomed flask, then palladium carbon with a mass fraction of 10% (5.65 g) was added, and an obtained mixture was subjected to hydrogen replacement for four times and stirred at room temperature for a reaction overnight. When all white solids were dissolved, the raw material CPD001-1 was completely consumed, and the reaction was stopped.

A reaction solution was directly filtered with a 200-300 mesh silica gel, and the silica gel was rinsed with dichloromethane until a filter cake had no obvious fluorescence. Silica gel column chromatography was conducted (a 200-300 mesh silica gel with petroleum ether as an eluting agent was used), and after elution was conducted, concentration was conducted to obtain a white solid, namely a compound CPD001-2 (27.42 g, purity: 99.99%, and yield: 95.77%). The mass spectrum was 291.37 (M+H).

### Synthesis of a Compound CPD001-3

The CPD001-2 (25.00 g, 86.07 mmol) and dichloromethane (450 ml) were added to a 1,000 ml three-mouth round-bottomed flask. Then, the system was cooled to -8° C. and below, and elemental iodine (1.09 g, 4.30 mmol) was added. Bromine (16.47 g, 103.29 mmol) was dissolved in dichloromethane (120 ml) and then slowly dropped into the reaction system, and heat preservation was conducted at -8° C. for a reaction for 5 hours. According to monitoring by TLC (with n-hexane as a developing agent), the raw material CPD001-2 was completely consumed, and the reaction was stopped.

A saturated sodium thiosulfate aqueous solution was dropped for quenching the reaction until a potassium iodide starch test paper was not turned to blue. A saturated sodium bicarbonate aqueous solution was added for adjusting the pH of the system to 8, and liquid separation was conducted. An organic phase was washed with deionized water (3*100 ml). Silica gel column chromatography was conducted (a 200-300 mesh silica gel with petroleum ether as an eluting agent was used), and after elution was conducted, concentration was conducted to obtain a yellow oily liquid, namely a compound CPD001-3 (31.31 g, purity: 99%, and yield: 98.5%). The mass spectrum was 369.15 (M+H).

### Synthesis of a Compound CPD001-4

The CPD001-3 (25.00 g, 67.69 mmol) and dried tetrahydrofuran (375 ml) were added to a 1,000 ml three-mouth round-bottomed flask, subjected to nitrogen replacement for four times, and then cooled to -78° C. An n-hexane solution containing 2.5 mol/1 of n-butyllithium (35.20 ml, 87.99 mmol) was dropped. After the dropping was completed within 1 hour, heat preservation was conducted at -78° C. for a reaction for 1 hour. The system was heated to -50° C. until the system was changed into a clarified solution, and a 2-bromofluorenone solid (21.05 g, 81.23 mmol) was directly added. The system was heated to -30° C. until the system was turned into brownish red, and then slowly heated to room temperature and stirred for a reaction overnight. According to monitoring of the reaction by TLC (with a mixture of ethyl acetate and n-hexane at a ratio of 1:50 as a developing agent), the raw materials CPD001-3 and 2-bromofluorenone were completely consumed.

A saturated ammonium chloride aqueous solution (200 ml) was added for quenching the reaction, the system was heated to room temperature, and concentration was conducted to remove the tetrahydrofuran. Dichloromethane (500 ml) and deionized water (300 ml) were added, and extraction was conducted for liquid separation. Purification was conducted by silica gel column chromatography (a 200-300 mesh silica gel with a mixture of tetrahydrofuran and petroleum ether at a ratio of 1:20 as an eluting agent), and then concentration was conducted to obtain a white-like solid, namely a compound CPD001-4 (22.85 g, purity: 99%, and yield: 61.43%). The mass spectrum was 547.27 (M-H).

### Synthesis of a Compound CPD001-5

The CPD001-4 (14.70 g, 25.94 mmol), acetic acid (160 ml), and 36%-38% of concentrated hydrochloric acid (16 ml) were added to a 250 ml one-mouth round-bottomed flask, heated to 90° C., and stirred for a reaction for 2 hours. According to monitoring by TLC (with a mixture of ethyl acetate and petroleum ether at a ratio of 1:40 as a developing agent), the raw material CPD001-4 was completely consumed.

The temperature was lowered to 60° C., ethanol (160 ml) was added, suction filtration was conducted, and a filter cake was rinsed with ethanol to obtain 14.35 g of a white-like solid. Toluene (70 ml) was added, heated to 100° C. for dissolved clarification, and cooled to 60° C. Methanol (110 ml) was dropped, cooled to room temperature, and stirred for 2 hours. Suction filtration was conducted, and then drying was conducted to obtain a white-like solid, namely a compound CPD001-5 (13.60 g, purity: 99.88%, and yield: 70.02%). The mass spectrum was 531.27 (M+H).

### Synthesis of a Compound CPD001

The CPD001-5 (7.65 g, 14.39 mmol), N-[1,1′-biphenyl]-2-yl-9,9-dimethyl-9H-fluorenyl-2-amine (5.40 g, 14.97 mmol), tri(dibenzylideneacetone)dipalladium (0.04 g, 0.43 mmol), sodium tert-butoxide (2.07 g, 21.59 mmol), and dried toluene (115 ml) were added to a 250 mL one-mouth round-bottomed flask, and subjected to nitrogen replacement for four times under stirring at room temperature. Then, a xylene solution containing 50% of tri-tert-butylphosphine (0.35 g, 0.86 mmol) was added under the protection of nitrogen, and heated to 110° C. for a reaction for 2 hours. According to monitoring of the reaction by TLC (with a mixture of toluene and petroleum ether at a ratio of 1:7 as a developing agent), the raw material CPD001-5 was completely consumed.

After the temperature was lowered to room temperature, toluene (250 ml) and deionized water (150 ml) were added, and extraction was conducted for liquid separation. Purification was conducted by silica gel column chromatography (a 200-300 mesh silica gel with a mixture of toluene and petroleum ether at a ratio of 1:20 as an eluting agent), and after elution was conducted, concentration was conducted to obtain a white solid, namely CPD001 (10.31 g, purity: 99.78%, and yield: 88.19%). 10.31 g of the crude product CPD001 was sublimated and purified to obtain a sublimated pure product CPD001 (8.8 g, purity: 99.94%, and yield: 85.35%). The mass spectrum was 834.01 (M+Na).

¹H NMR (400 MHz, CDCl₃) δ 7.72(d, J = 7.6 Hz, 1H), 7.60 (d, J=8.3 Hz, 1H), 7.56 (d, J= 7.9 Hz, 2H), 7.50 (d, J= 7.3 Hz, 1H), 7.35-7.26 (m, 6H), 7.24-7.15 (m, 7H), 7.03-6.97 (m, 4H), 6.88 (d, J= 8.3 Hz, 1H), 6.76 (s, 1H), 6.65 (d, J= 7.6 Hz, 1H), 6.60 (m, 4H), 2.93-2.85 (m, 2H), 2.00 (m, 4H), 1.78 (m, 4H), 1.67-1.64(m, 4H), 1.52 (m, 4H), 1.00 (s, 6H).

### Synthesis of a Compound CPD003

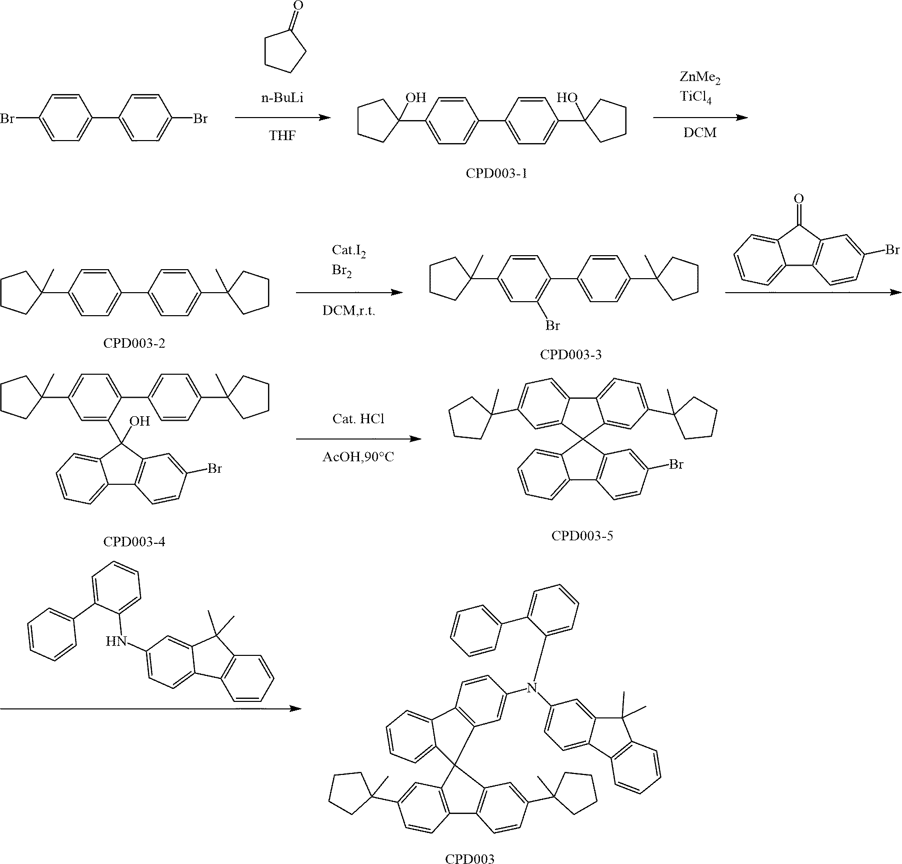

### Synthesis of a Compound CPD003-1

4,4′-dibromobiphenyl (20 g, 64.10 mmol) and dried tetrahydrofuran (300 ml) were added to a 1,000 ml three-mouth round-bottomed flask, subjected to nitrogen replacement for four times, and then cooled to -78° C. with liquid nitrogen. An n-hexane solution containing 2.5 mol/1 of n-butyllithium (64.10 ml, 160.25 mmol) was dropped. After the dropping was completed within 1 hour, heat preservation was conducted at -78° C. for a reaction for 1 hour. Cyclopentanone (13.48 g, 160.25 mmol) was directly added, and the dropping was completed within 15 minutes. According to monitoring by TLC (with a mixture of ethyl acetate and petroleum ether at a ratio of 1:5) for 1 hour, the raw material 4,4′-dibromobiphenyl was completely consumed, and most of CPD003-1 was produced.

A saturated ammonium chloride aqueous solution (200 ml) was added for quenching the reaction at a temperature maintained -78° C., the system was heated to room temperature, and concentration was conducted to remove the tetrahydrofuran. Dichloromethane (500 ml) and deionized water (300 ml) were added, and extraction was conducted for liquid separation. Purification was conducted by silica gel column chromatography (a 200-300 mesh silica gel with a mixture of ethyl acetate and petroleum ether at a ratio of 1:40 as an eluting agent), and then concentration was conducted to obtain a white solid, namely a compound CPD003-1 (13.44 g, purity: 99.5 %, and yield: 65.00%). The mass spectrum was 323.08 (M-H).

### Synthesis of a Compound CPD003-2

Titanium tetrachloride (23.65, 124.67 mmol) and dried dichloromethane (200 ml) were added to a 500 ml dried three-mouth round-bottomed flask, and subjected to nitrogen replacement for four times. Then, the system was cooled to 0° C. under stirring. A toluene solution containing 2 mol/1 of dimethyl zinc (11.90 g, 124.67 mmol) was added, the dropping was completed within 20 minutes, and a reaction was conducted at a temperature maintained 0° C. for 30 minutes.

The CPD003-1 (13.40 g, 41.56 mmol) was dissolved in dried dichloromethane (268 ml) and then dropped into the system at 0° C. After the dropping was completed within 30 minutes, the system was naturally heated to room temperature and stirred overnight. According to monitoring by TLC (with a mixture of ethyl acetate and petroleum ether at a ratio of 1:9), the raw material CPD003-1 was completely consumed.

The system was cooled to 0° C., deionized water (100 ml) was added for quenching the reaction, and liquid separation was conducted. An organic phase was washed with deionized water (3*150 ml). Silica gel column chromatography was conducted (a 200-300 mesh silica gel with petroleum ether as an eluting agent was used), and after elution was conducted, concentration was conducted to obtain a white solid, namely a compound CPD003-2 (9.58 g, purity: 99.9%, and yield: 72.38%). The mass spectrum was 319.54 (M+H).

### Synthesis of a Compound CPD003-3

With reference to the synthesis and purification methods of the compound CPD001-3, only the corresponding raw materials were required to be changed, and a target compound CPD003-3 (20.87 g, purity: 99.20%, and yield: 78.05%) was obtained. The mass spectrum was 397.84 (M+H).

### Synthesis of a Compound CPD003-4

With reference to the synthesis and purification methods of the compound CPD001-4, only the corresponding raw materials were required to be changed, and a target compound CPD003-4 (17.50 g, purity: 99.10%, and yield: 68.01%) was obtained. The mass spectrum was 575.19 (M+H).

### Synthesis of a Compound CPD003-5

With reference to the synthesis and purification methods of the compound CPD001-5, only the corresponding raw materials were required to be changed, and a target compound CPD003-5 (15.30 g, purity: 99.75%, and yield: 75.05%) was obtained. The mass spectrum was 559.23 (M+H).

### Synthesis of a Compound CPD003

With reference to the synthesis and purification methods of the compound CPD001, only the corresponding raw materials were required to be changed, and a white solid, namely a target compound CPD003 (11.80 g, purity: 99.90%, and yield: 83.20%) was obtained. 11.8 g of the crude product CPD003 was sublimated and purified to obtain a sublimated pure product CPD003 (9.20 g, purity: 99.94%, and yield: 77.96%). The mass spectrum was 862.55 (M+Na).

¹H NMR (400 MHz, CDCl₃) δ 7.71(d, J = 7.6 Hz, 1H), 7.58 (d, J=8.2 Hz, 1H), 7.53 (d, J = 7.7 Hz, 2H), 7.48-7.41 (m, 1H), 7.34-7.26 (m, 6H), 7.23-7.12 (m, 6H), 7.00-6.90 (m, 6H), 6.80-6.66 (m, 6H), 2.04 (m, 4H), 1.76(m, 4H), 1.68-1.66(m, 4H), 1.54 (m, 4H), 1.35(s, 6H), 1.02 (s, 6H).

### Synthesis of a Compound CPD005

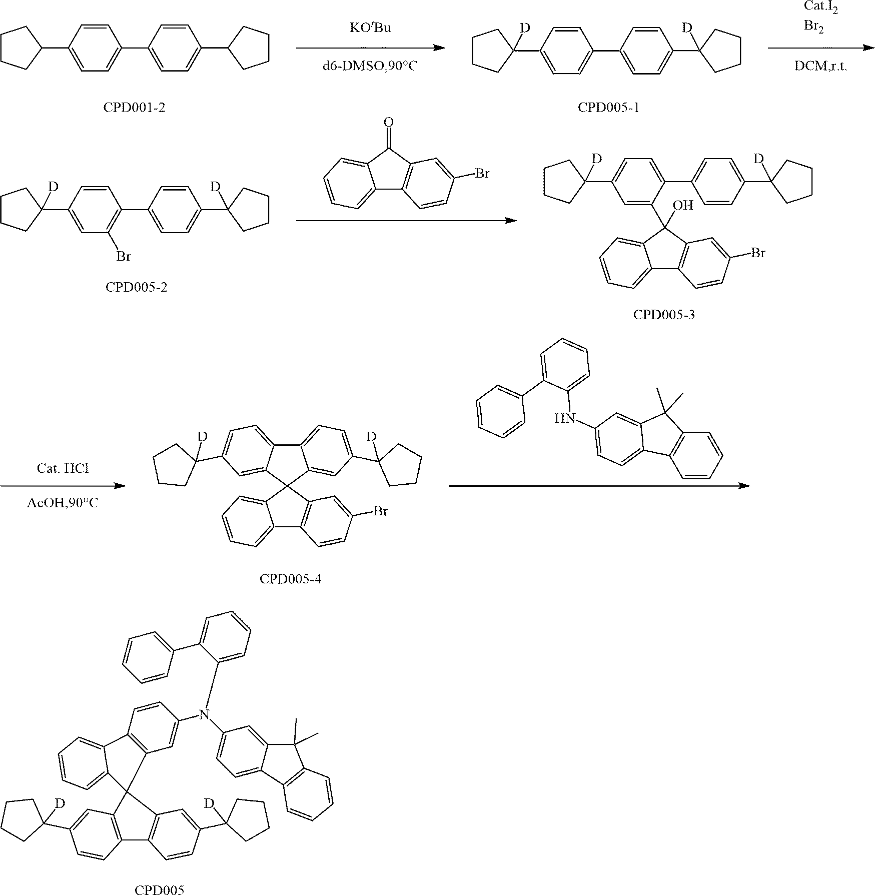

### Synthesis of a Compound CPD005-1

The CPD001-2 (50 g, 172.14 mmol), deuterated dimethyl sulfoxide (250 ml), and potassium tert-butoxide (57.95 g, 516.44 mmol) were added to a 500 ml three-mouth round-bottomed flask, subjected to nitrogen replacement for four times, and then heated to 90° C. for a reaction for 24 hours. According to monitoring by nuclear magnetic resonance and mass spectrum, the deuterization rate at a benzyl position was 99% or above, and the heating was stopped.

Deionized water (500 ml) was added to the system for precipitating out a solid, and suction filtration was conducted. A filter cake was washed with deionized water (300 ml) and then dried at 80° C. to obtain a white solid, namely CPD005-1 (45.91 g, purity: 99.9%, deuterization rate: 99%, and yield: 91.20%). The mass spectrum was 293.43 (M+H).

### Synthesis of a Compound CPD005-2

With reference to the synthesis and purification methods of the compound CPD001-3, only the corresponding raw materials were required to be changed, and a target compound CPD005-2 (43.72 g, purity: 99.42%, and yield: 75.05%) was obtained. The mass spectrum was 371.23 (M+H).

### Synthesis of a Compound CPD005-3

With reference to the synthesis and purification methods of the compound CPD001-4, only the corresponding raw materials were required to be changed, and a target compound CPD005-3 (42.59 g, purity: 99.12%, and yield: 65.61%) was obtained. The mass spectrum was 549.26 (M+H).

### Synthesis of a Compound CPD005-4

With reference to the synthesis and purification methods of the compound CPD001-5, only the corresponding raw materials were required to be changed, and a target compound CPD005-4 (40.11 g, purity: 99.76%, and yield: 75.17%) was obtained. The mass spectrum was 533.28 (M+H).

### Synthesis of a Compound CPD005

With reference to the synthesis and purification methods of the compound CPD001, only the corresponding raw materials were required to be changed, and a white solid, namely a target compound CPD005 (32.12 g, purity: 99.92%, and yield: 83.20%) was obtained. 32.12 g of the crude product CPD005 was sublimated and purified to obtain a sublimated pure product CPD005 (24.16 g, purity: 99.95%, deuterization rate: 99% or above, and yield: 75.23%). The mass spectrum was 836.15 (M+Na).

¹H NMR (400 MHz, CDCl₃) δ 7.67-7.42 (m, 2H), 7.58 (d, J=7.4 Hz, 1H), 7.54-7.47 (m, 4H), 7.36-7.27 (m, 1H), 7.24-7.13 (m, 2H), 7.04-6.94 (m, 11H), 6.87-6.76 (m, 5H), 6.72-6.62 (m, 3H), 2.00 (m, 4H), 1.77 (m, 4H), 1.67-1.63 (m, 4H), 1.52 (m, 4H), 1.01 (s, 6H).

### Synthesis of a Compound CPD007

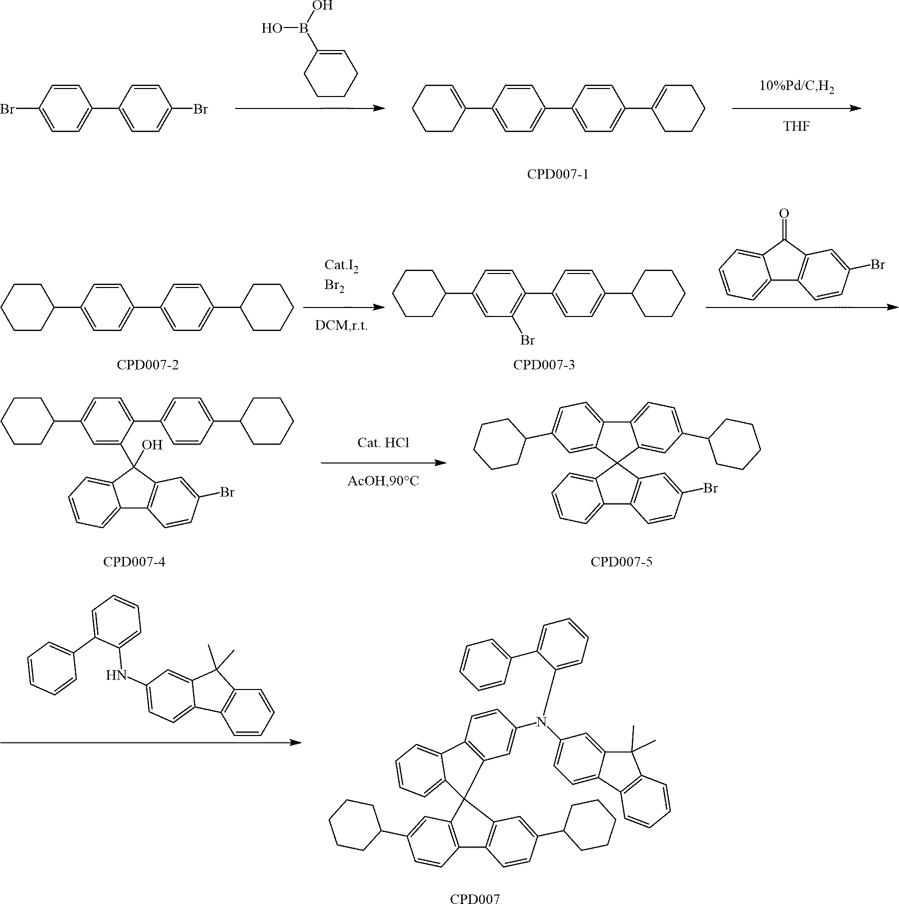

### Synthesis of a Compound CPD007-1

With reference to the synthesis and purification methods of the compound CPD001-1, only the corresponding raw materials were required to be changed, and a target compound CPD007-1 (45.83 g, purity: 99.83%, and yield: 93.31%) was obtained. The mass spectrum was 315.23 (M+H).

### Synthesis of a Compound CPD007-2

With reference to the synthesis and purification methods of the compound CPD001-2, only the corresponding raw materials were required to be changed, and a target compound CPD007-2 (44.14 g, purity: 99.9%, and yield: 95.11%) was obtained. The mass spectrum was 319.49 (M+H).

### Synthesis of a Compound CPD007-3

With reference to the synthesis and purification methods of the compound CPD001-3, only the corresponding raw materials were required to be changed, and a target compound CPD007-3 (53.70 g, purity: 99.30%, and yield: 97.52%) was obtained. The mass spectrum was 397.28 (M+H).

### Synthesis of a Compound CPD007-4

With reference to the synthesis and purification methods of the compound CPD001-4, only the corresponding raw materials were required to be changed, and a target compound CPD007-4 (47.33 g, purity: 99.00%, and yield: 62.82%) was obtained. The mass spectrum was 575.21 (M+H).

### Synthesis of a Compound CPD007-5

With reference to the synthesis and purification methods of the compound CPD001-5, only the corresponding raw materials were required to be changed, and a target compound CPD007-5 (31.43 g, purity: 99.9%, and yield: 68.56%) was obtained. The mass spectrum was 560.57 (M+H).

### Synthesis of a Compound CPD007

With reference to the synthesis and purification methods of the compound CPD001, only the corresponding raw materials were required to be changed, and a white solid, namely a target compound CPD007 (37.22 g, purity: 99.91%, and yield: 78.88%) was obtained. 37.22 g of the crude product CPD007 was sublimated and purified to obtain a sublimated pure product CPD007 (29.85 g, purity: 99.98%, and yield: 80.20%). The mass spectrum was 863.07 (M+Na).

¹H NMR (400 MHz, CDCl₃) δ 7.71-7.58 (m, 2H), 7.55 (d, J= 7.9 Hz, 2H), 7.50 (d, J= 7.3 Hz, 1H), 7.35-7.26 (m, 6H), 7.24-7.15 (m, 6H), 7.03-6.88 (m, 6H), 6.76-6.60 (m, 6H), 2.67-2.6(m,2H), 1.97-1.81 (m, 8H), 1.68-1.55 (m, 12H), 1.03 (s, 6H).

### Synthesis of a Compound CPD008

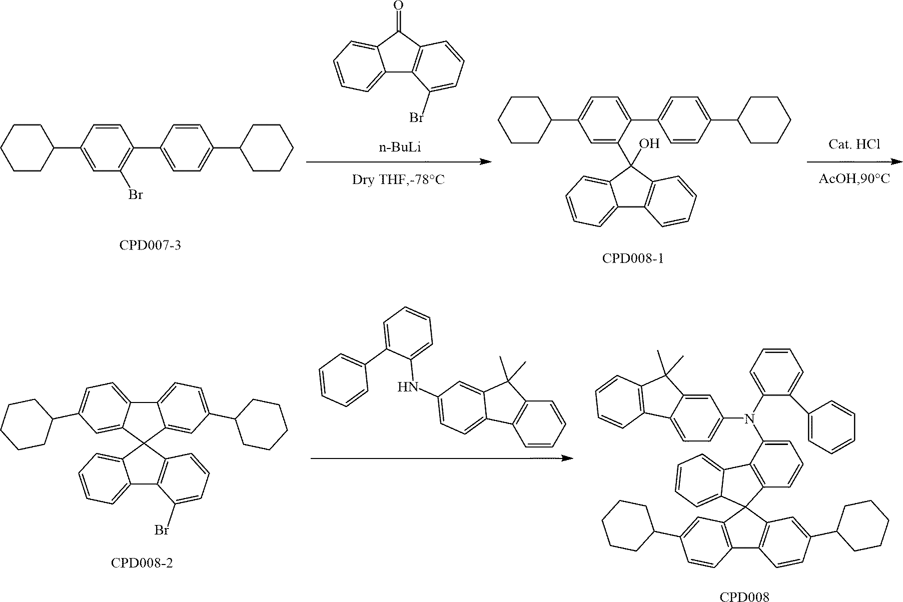

### Synthesis of a Compound CPD008-1

With reference to the synthesis and purification methods of the compound CPD001-4, only the corresponding raw materials were required to be changed, and a target compound CPD008-1 (26.23 g, purity: 98.1 %, and yield: 65.10%) was obtained. The mass spectrum was 497.28 (M+H).

### Synthesis of a Compound CPD008-2

With reference to the synthesis and purification methods of the compound CPD001-5, only the corresponding raw materials were required to be changed, and a target compound CPD008-2 (18.02 g, purity: 99.57 %, and yield: 68.73%) was obtained. The mass spectrum was 560.58 (M+H).

### Synthesis of a Compound CPD008

With reference to the synthesis and purification methods of the compound CPD001, only the corresponding raw materials were required to be changed, and a target compound CPD008 (21.90 g, purity: 99.97 %, and yield: 80.97%) was obtained. 21.90 g of the crude product CPD008 was sublimated and purified to obtain a sublimated pure product CPD008 (16.56 g, purity: 99.97%, and yield: 75.63%). The mass spectrum was 863.07 (M+Na).

¹H NMR (400 MHz, CDCl₃) δ 7.71-7.68 (m, 2H), 7.52-7.51(m, 2H), 7.49-7.48 (m, 2H), 7.24-7.13 (m, 4H), 7.06-6.94 (m, 9H), 6.91-6.80 (m, 6H), 6.77-6.60 (m, 4H), 2.68-2.57(m,2H), 1.92- 1.78 (m, 8H), 1.70-1.60 (m, 12H), 1.04 (s, 6H).

### Synthesis of a Compound CPD019

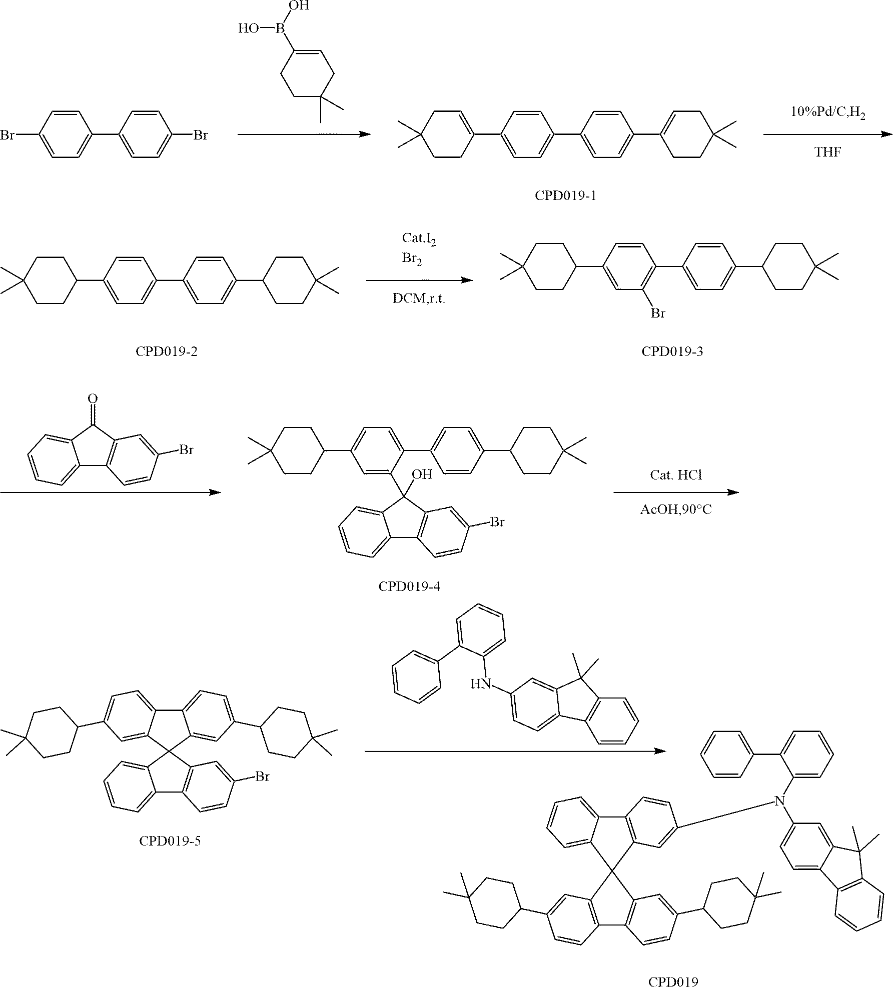

### Synthesis of a Compound CPD019-1

With reference to the synthesis and purification methods of the compound CPD001-1, only the corresponding raw materials were required to be changed, and a target compound CPD019-1 (38.52 g, purity: 99.75%, and yield: 92.81%) was obtained. The mass spectrum was 371.38 (M+H).

### Synthesis of a Compound CPD019-2

With reference to the synthesis and purification methods of the compound CPD001-2, only the corresponding raw materials were required to be changed, and a target compound CPD019-2 (33.79 g, purity: 99.91%, and yield: 93.34%) was obtained. The mass spectrum was 375.31 (M+H).

### Synthesis of a Compound CPD019-3

With reference to the synthesis and purification methods of the compound CPD001-3, only the corresponding raw materials were required to be changed, and a target compound CPD019-3 (36.82 g, purity: 99.14%, and yield: 90.01%) was obtained. The mass spectrum was 453.43 (M+H).

### Synthesis of a Compound CPD019-4

With reference to the synthesis and purification methods of the compound CPD001-4, only the corresponding raw materials were required to be changed, and a target compound CPD019-4 (31.26 g, purity: 99.00%, and yield: 60.76%) was obtained. The mass spectrum was 631.74 (M+H).

### Synthesis of a Compound CPD019-5

With reference to the synthesis and purification methods of the compound CPD001-5, only the corresponding raw materials were required to be changed, and a target compound CPD019-5 (19.90 g, purity: 99.91%, and yield: 65.55%) was obtained. The mass spectrum was 615.25 (M+H).

### Synthesis of a Compound CPD019

With reference to the synthesis and purification methods of the compound CPD001, only the corresponding raw materials were required to be changed, and a white solid, namely a target compound CPD019 (24.15 g, purity: 99.93%, and yield: 83.37%) was obtained. 24.15 g of the crude product CPD019 was sublimated and purified to obtain a sublimated pure product CPD019 (18.96 g, purity: 99.96%, and yield: 78.53%). The mass spectrum was 919.05 (M+Na).

¹H NMR (400 MHz, CDCl₃) δ 7.72-7.58 (m, 2H), 7.55-7.51 (m, 3H), 7.36-7.27 (m, 6H), 7.25-7.16 (m, 6H), 7.03-6.98 (m, 6H), 6.86-6.70 (m, 6H), 2.80-2.73(m,2H), 1.96-1.82 (m, 8H), 1.65-1.60 (m, 8H), 1.10(s, 12H), 1.03 (s, 6H).

### Synthesis of a Compound CPD039

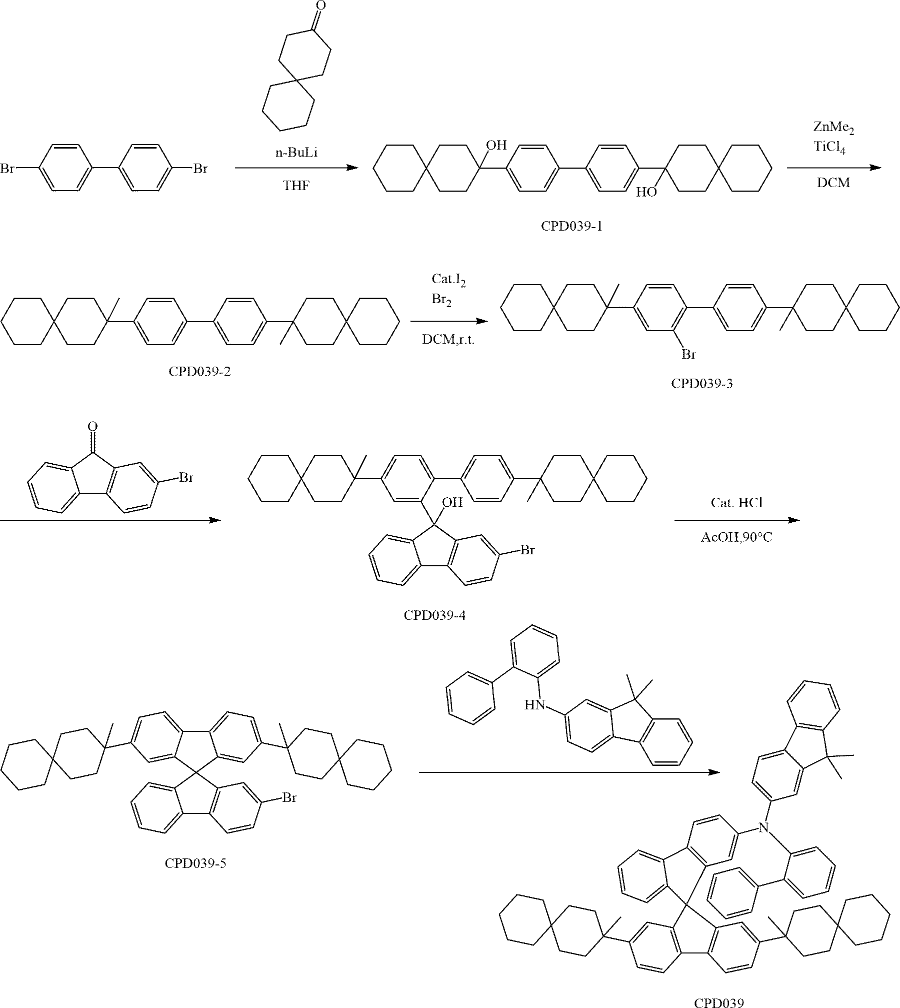

### Synthesis of a Compound CPD039-1

With reference to the synthesis and purification methods of the compound CPD003-1, only the corresponding raw materials were required to be changed, and a target compound CPD039-1 (21.22 g, purity: 99.31%, and yield: 68.01%) was obtained. The mass spectrum was 487.25 (M+H).

### Synthesis of a Compound CPD039-2

With reference to the synthesis and purification methods of the compound CPD003-2, only the corresponding raw materials were required to be changed, and a target compound CPD039-2 (15.79 g, purity: 99.80%, and yield: 75.13%) was obtained. The mass spectrum was 483.28 (M+H).

### Synthesis of a Compound CPD039-3

With reference to the synthesis and purification methods of the compound CPD001-3, only the corresponding raw materials were required to be changed, and a target compound CPD039-3 (17.46 g, purity: 99.23%, and yield: 95.42%) was obtained. The mass spectrum was 561.63 (M+H).

### Synthesis of a Compound CPD039-4

With reference to the synthesis and purification methods of the compound CPD001-4, only the corresponding raw materials were required to be changed, and a target compound CPD039-4 (15.07 g, purity: 98.90%, and yield: 65.35%) was obtained. The mass spectrum was 739.35 (M+H).

### Synthesis of a Compound CPD039-5

With reference to the synthesis and purification methods of the compound CPD001-5, only the corresponding raw materials were required to be changed, and a target compound CPD039-5 (11.04 g, purity: 99.61%, and yield: 75.07%) was obtained. The mass spectrum was 723.25 (M+H).

### Synthesis of a Compound CPD039

With reference to the synthesis and purification methods of the compound CPD001, only the corresponding raw materials were required to be changed, and a white solid, namely a target compound CPD039 (13.58 g, purity: 99.96%, and yield: 88.65%) was obtained. 13.58 g of the crude product CPD039 was sublimated and purified to obtain a sublimated pure product CPD039 (10.21 g, purity: 99.96%, and yield: 75.22%). The mass spectrum was 1026.86 (M+Na).

¹H NMR (400 MHz, CDCl₃) δ 7.70(d, J = 7.56 Hz, 1H), 7.57 (d, J=8.3 Hz, 1H), 7.53-7.42 (m, 3H), 7.35-7.24 (m, 6H), 7.23-7.12 (m, 6H), 7.00-6.90 (m, 8H), 6.80-6.66 (m, 4H), 2.08(s, 6H), 1.83(m, 16H), 1.65(m, 4H), 1.52-1.5(m, 10H), 1.50-41.42(m, 6H), 1.04 (s, 6H).

### Synthesis of a Compound CPD049

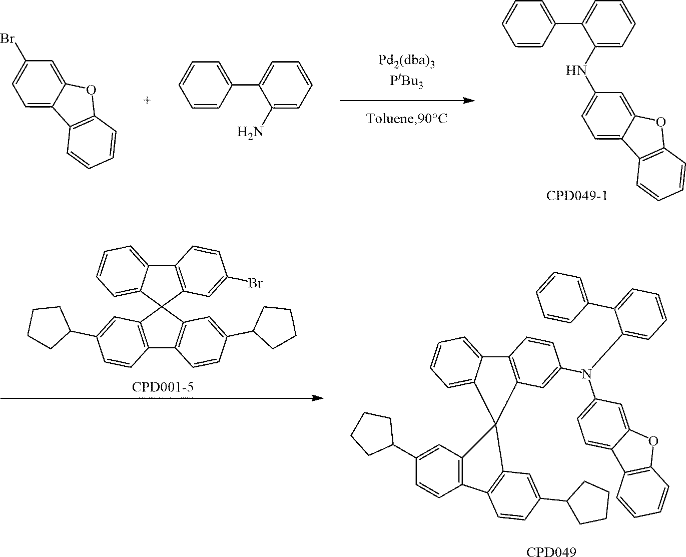

### Synthesis of a Compound CPD049-1

3-bromodibenzofuran (40.00 g, 161.88 mmol), 2-aminodiphenyl (32.87 g, 194.26 mmol), tri(dibenzylideneacetone)dipalladium (1.48 g, 1.62 mmol), sodium tert-butoxide (23.34 g, 242.88 mmol), and dried toluene (400 ml) were added to a 1,000 mL one-mouth round-bottomed flask, and subjected to nitrogen replacement for four times under stirring at room temperature. Then, a xylene solution containing 50% of tri-tert-butylphosphine (1.31 g, 3.24 mmol) was added under the protection of nitrogen, and heated to 90° C. for a reaction for 1 hour. According to monitoring of the reaction by TLC (with a mixture of ethyl acetate and petroleum ether at a ratio of 1:8 as a developing agent), the raw material 3-bromodibenzofuran was completely consumed.

After the temperature was lowered to room temperature, deionized water (3 * 150 ml) was added for washing, and liquid separation and concentration were conducted. Purification was conducted by silica gel column chromatography (a 200-300 mesh silica gel with a mixture of ethyl acetate and petroleum ether at a ratio of 1:20 as an eluting agent), and after elution was conducted, concentration was conducted to obtain a white solid, namely CPD049-1 (48.98 g, purity: 99.56%, and yield: 90.21%). The mass spectrum was 336.42 (M+H).

### Synthesis of a Compound CPD049

With reference to the synthesis and purification methods of the compound CPD001, only the corresponding raw materials were required to be changed, and a white solid, namely a target compound CPD049 (31.65 g, purity: 99.97%, and yield: 82.33%) was obtained. 31.65 g of the crude product CPD049 was sublimated and purified to obtain a sublimated pure product CPD049 (23.00 g, purity: 99.98%, and yield: 72.67%). The mass spectrum was 809.13 (M+Na).

¹H NMR (400 MHz, CDCl₃) δ 7.93(d, J = 7.86 Hz, 2H), 7.75-7.72(m, 2H), 7.68-7.53 (m, 4H), 7.37-7.22 (m, 6H), 7.20-7.12 (m, 8H), 7.03-6.97 (m, 4H), 6.75(m, 3H), 3.10-2.93 (m, 2H), 2.10 (m, 4H), 1.78 (m, 4H), 1.68 (m, 4H), 1.52 (m, 4H).

### Synthesis of a Compound CPD061

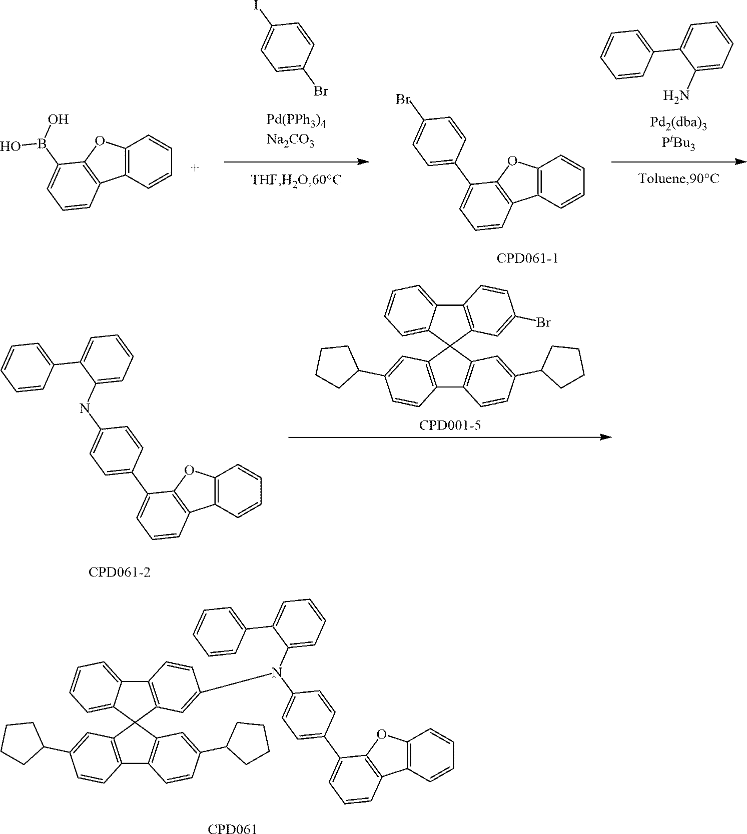

### Synthesis of a Compound CPD061-1

4-dibenzofuranoboric acid (30.00 g, 141.50 mmol), p-bromiodobenzene (48.04 g, 169.80 mmol), tetra(triphenylphosphine)palladium (8.18 g, 7.08 mmol), sodium carbonate (29.99 g, 283.00 mmol), deionized water (141 ml), and tetrahydrofuran (500 ml) were added to a 1,000 mL one-mouth round-bottomed flask, and subjected to nitrogen replacement for four times under stirring at room temperature for a reaction at 60° C. overnight. According to monitoring of the reaction by TLC (with a mixture of ethyl acetate and petroleum ether at a ratio of 1:20 as a developing agent), the raw material 4-dibenzofuranoboric acid was completely consumed.

After the temperature was lowered to room temperature, deionized water (3*120 ml) was added for washing, and liquid separation and concentration were conducted. Purification was conducted by silica gel column chromatography (a 200-300 mesh silica gel with a mixture of ethyl acetate and petroleum ether at a ratio of 1:50 as an eluting agent), and after elution was conducted, concentration was conducted to obtain a white solid, namely CPD061-1 (32.01 g, purity: 99.51%, and yield: 70.00%). The mass spectrum was 323.02 (M+H).

### Synthesis of a Compound CPD061-2

With reference to the synthesis and purification methods of the compound CPD049-1, only the corresponding raw materials were required to be changed, and a target compound CPD061-2 (34.77 g, purity: 99.70 %, and yield: 85.54%) was obtained. The mass spectrum was 411.19 (M+H).

### Synthesis of a Compound CPD061

With reference to the synthesis and purification methods of the compound CPD001, only the corresponding raw materials were required to be changed, and a white solid, namely a target compound CPD061 (31.20 g, purity: 99.93%, and yield: 81.73%) was obtained. 31.20 g of the crude product CPD061 was sublimated and purified to obtain a sublimated pure product CPD061 (23.62 g, purity: 99.93%, and yield: 75.72%). The mass spectrum was 884.56 (M+Na).

¹H NMR (400 MHz, CDCl₃) δ 8.02(d, J = 7.86 Hz, 2H), 7.86-7.72(m, 2H), 7.63-7.42 (m, 8H), 7.37-7.22 (m, 6H), 7.20-7.12 (m, 6H), 7.03-6.97 (m, 6H), 6.75 (m, 3H), 3.15-3.02 (m, 2H), 2.21 (m, 4H), 1.88 (m, 4H), 1.78 (m, 4H), 1.62 (m, 4H).

### Synthesis of a Compound CPD073

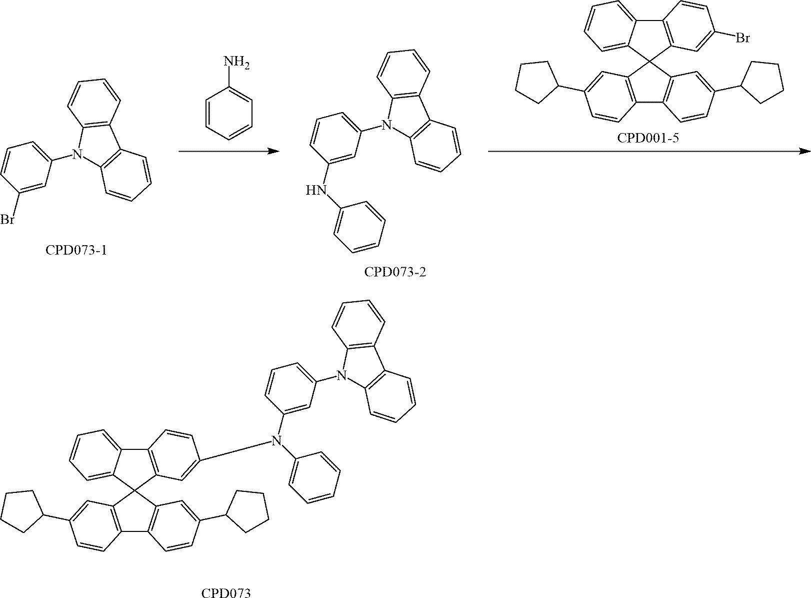

### Synthesis of a Compound CPD073-2

With reference to the synthesis and purification methods of the compound CPD049-1, only the corresponding raw materials were required to be changed, and a target compound CPD073-2 (22.70 g, purity: 99.63 %, and yield: 83.45%) was obtained. The mass spectrum was 335.45 (M+H).

### Synthesis of a Compound CPD073

With reference to the synthesis and purification methods of the compound CPD001, only the corresponding raw materials were required to be changed, and a white solid, namely a target compound CPD073 (27.98 g, purity: 99.94%, and yield: 85.14%) was obtained. 27.98 g of the crude product CPD073 was sublimated and purified to obtain a sublimated pure product CPD073 (20.22 g, purity: 99.95%, and yield: 72.27%). The mass spectrum was 808.05 (M+Na).

¹H NMR (400 MHz, CDCl₃) δ 8.14(d, J= 7.8 Hz, 2H), 7.79(m, 2H), 7.50-7.46 (m, 8H), 7.28 (m, 2H), 7.17-7.09 (m, 6H), 7.03-6.94 (m, 6H), 6.74(m, 4H), 2.90-3.87 (m, 2H), 2.32-1.98 (m, 8H), 1.86-1.62 (m, 8H).

### Synthesis of a Compound CPD097

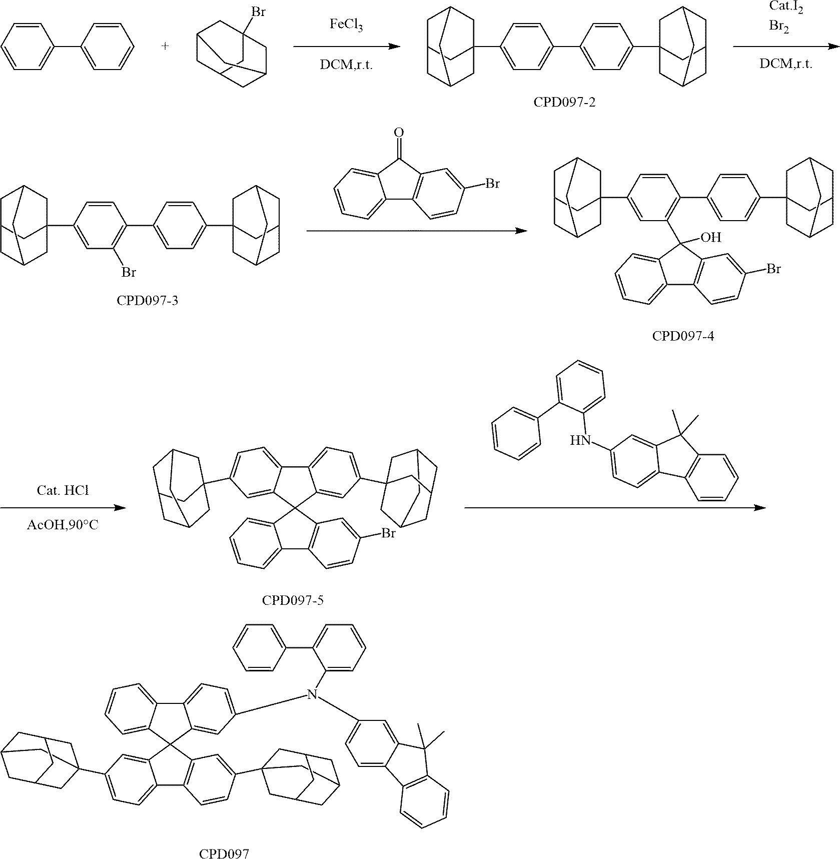

### Synthesis of a Compound CPD097-2

Biphenyl (20.00 g, 129.69 mmol), anhydrous ferric chloride (2.10 g, 12.97 mmol), and dichloromethane (200 ml) were added to a 2,000 ml three-mouth round-bottomed flask and stirred at room temperature. Then, 1-bromoadamantane (58.59 g, 272.35 mmol) was dissolved in dichloromethane (580 ml), and dropped to the above reaction system. After the dropping was completed within 45 minutes, the system was stirred overnight at room temperature. According to monitoring of a reaction by TLC (with petroleum ether as a developing agent), the raw material biphenyl was completely consumed.

Deionized water (3*300 ml) was added for washing, and extraction for liquid separation and concentration were conducted. Purification was conducted by silica gel column chromatography (a 200-300 mesh silica gel with a mixture of ethyl acetate and petroleum ether at a ratio of 1:20 as an eluting agent), and after elution was conducted, concentration was conducted to obtain CPD097-2 (44.05 g, purity: 99.73%, and yield: 80.37%). The mass spectrum was 423.21 (M+H).

### Synthesis of a Compound CPD097-3

With reference to the synthesis and purification methods of the compound CPD001-3, only the corresponding raw materials were required to be changed, and a target compound CPD097-3 (46.18 g, purity: 99.18 %, and yield: 88.35%) was obtained. The mass spectrum was 501.52 (M+H).

### Synthesis of a Compound CPD097-4

With reference to the synthesis and purification methods of the compound CPD001-4, only the corresponding raw materials were required to be changed, and a target compound CPD097-4 (39.81 g, purity: 99.3%, and yield: 63.42%) was obtained. The mass spectrum was 679.26 (M+H).

### Synthesis of a Compound CPD097-5

With reference to the synthesis and purification methods of the compound CPD001-5, only the corresponding raw materials were required to be changed, and a target compound CPD097-5 (30.23 g, purity: 99.72%, and yield: 78.00%) was obtained. The mass spectrum was 663.15 (M+H).

### Synthesis of a Compound CPD097

With reference to the synthesis and purification methods of the compound CPD001, only the corresponding raw materials were required to be changed, and a white solid, namely a target compound CPD097 (21.76 g, purity: 99.93%, and yield: 76.46%) was obtained. 21.76 g of the crude product CPD097 was sublimated and purified to obtain a sublimated pure product CPD097 (14.97 g, purity: 99.94%, and yield: 68.83%). The mass spectrum was 967.24 (M+Na).

¹H NMR (400 MHz, CDCl₃) δ7.73(d, J= 7.7 Hz, 2H), 7.69-7.60 (m, 3H), 7.48 (m, 2H), 7.32-7.19 (m, 6H), 7.18-6.93 (m, 10H), 6.88-6.63 (m, 6H), 1.81-1.78 (m, 15H), 1.51-1.48 (m, 15H), 1.03(s, 6H).

### Synthesis of a Compound CPD106

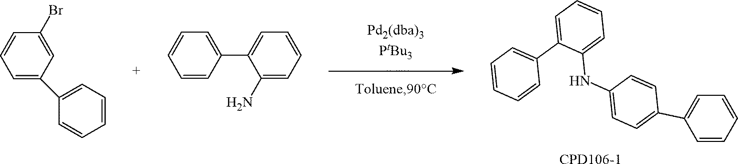

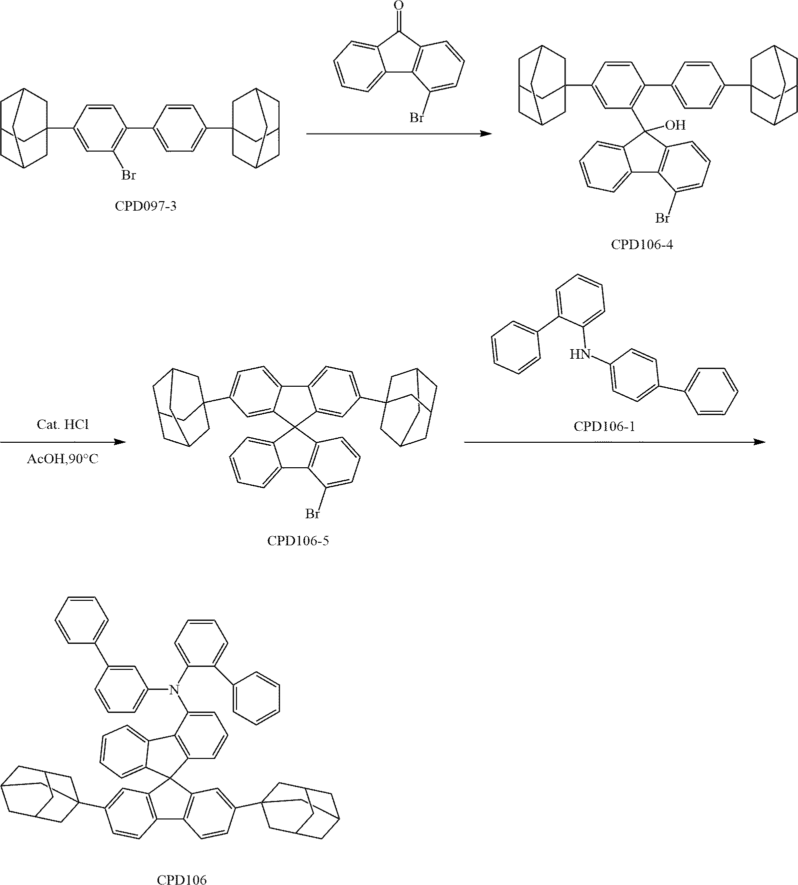

### Synthesis of a Compound CPD106-1

With reference to the synthesis and purification methods of the compound CPD049-1, only the corresponding raw materials were required to be changed, and a target compound CPD106-1 (37.32 g, purity: 99.70%, and yield: 90.21%) was obtained. The mass spectrum was 322.24 (M+H).

### Synthesis of a Compound CPD106-4

With reference to the synthesis and purification methods of the compound CPD001-4, only the corresponding raw materials were required to be changed, and a target compound CPD106-4 (17.67 g, purity: 99.45%, and yield: 65.00%) was obtained. The mass spectrum was 679.26 (M+H).

### Synthesis of a Compound CPD106-5

With reference to the synthesis and purification methods of the compound CPD001-5, only the corresponding raw materials were required to be changed, and a target compound CPD106-5 (12.96 g, purity: 99.80%, and yield: 75.35%) was obtained. The mass spectrum was 663.15 (M+H).

### Synthesis of a Compound CPD106

With reference to the synthesis and purification methods of the compound CPD001, only the corresponding raw materials were required to be changed, and a white solid, namely a target compound CPD106 (27.59 g, purity: 99.95%, and yield: 78.25%) was obtained. 27.59 g of the crude product CPD106 was sublimated and purified to obtain a sublimated pure product CPD106 (19.13 g, purity: 99.95%, and yield: 69.37%). The mass spectrum was 926.78 (M+Na).

¹H NMR (400 MHz, CDCl₃) δ 7.75(m, 4H), 7.19-6.99(m, 11H), 6.91-6.78 (m, 10H), 6.72 (m, 6H), 1.83-1.78 (m, 15H), 1.54-1.50 (m, 15H).

### Synthesis of a Compound CPD117

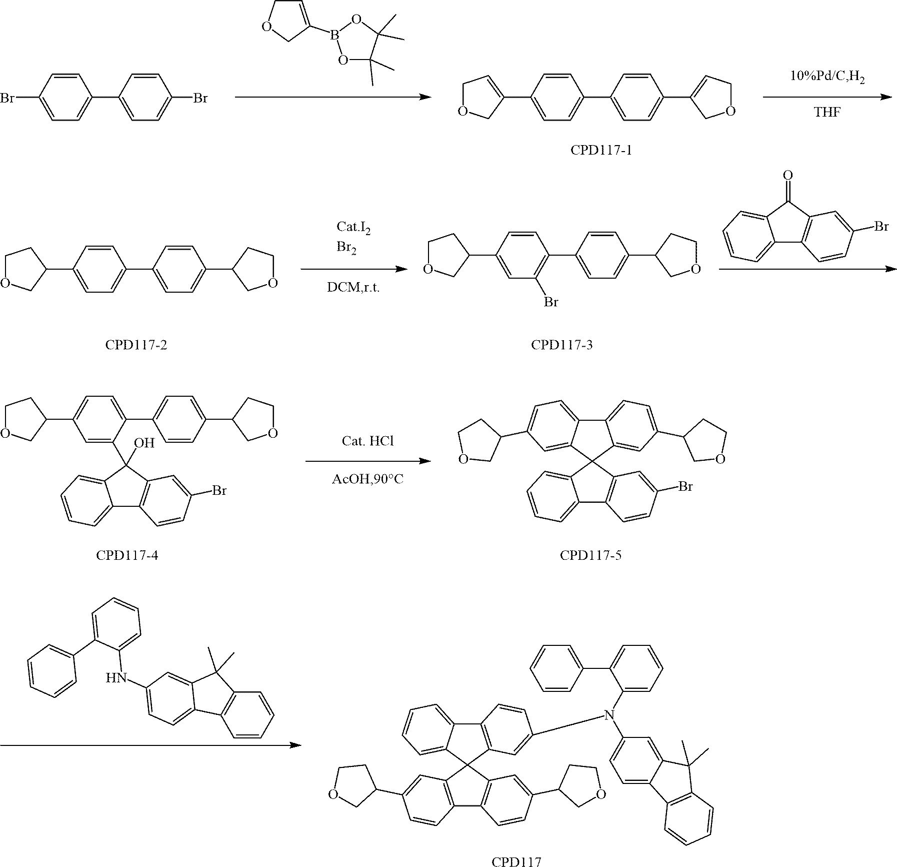

### Synthesis of a Compound CPD117-1

With reference to the synthesis and purification methods of the compound CPD001-1, only the corresponding raw materials were required to be changed, and a target compound CPD117-1 (19.89 g, purity: 99.33%, and yield: 85.51%) was obtained. The mass spectrum was 291.23 (M+H).

### Synthesis of a Compound CPD117-2

With reference to the synthesis and purification methods of the compound CPD001-2, only the corresponding raw materials were required to be changed, and a target compound CPD117-2 (19.49 g, purity: 99.85%, and yield: 96.63%) was obtained. The mass spectrum was 295.17 (M+H).

### Synthesis of a Compound CPD117-3

With reference to the synthesis and purification methods of the compound CPD001-3, only the corresponding raw materials were required to be changed, and a target compound CPD117-3 (23.54 g, purity: 99.01%, and yield: 95.25%) was obtained. The mass spectrum was 373.06 (M+H).

### Synthesis of a Compound CPD117-4

With reference to the synthesis and purification methods of the compound CPD001-4, only the corresponding raw materials were required to be changed, and a target compound CPD117-4 (23.83 g, purity: 99.13%, and yield: 68.26%) was obtained. The mass spectrum was 551.50 (M+H).

### Synthesis of a Compound CPD 117-5

With reference to the synthesis and purification methods of the compound CPD001-5, only the corresponding raw materials were required to be changed, and a target compound CPD117-5 (16.95 g, purity: 99.87%, and yield: 73.53%) was obtained. The mass spectrum was 535.21 (M+H).

### Synthesis of a Compound CPD117

With reference to the synthesis and purification methods of the compound CPD001, only the corresponding raw materials were required to be changed, and a white solid, namely a target compound CPD117 (18.01 g, purity: 99.97%, and yield: 78.80%) was obtained. 18.01 g of the crude product CPD117 was sublimated and purified to obtain a sublimated pure product CPD117 (11.84 g, purity: 99.97%, and yield: 65.75%). The mass spectrum was 839.01 (M+Na).

¹H NMR (400 MHz, CDCl₃) δ 7.71(d, J = 7.62 Hz, 1H), 7.58 (d, J=8.33 Hz, 1H), 7.56 (d, J = 7.9 Hz, 2H), 7.51-7.25 (m, 7H), 7.24-7.15 (m, 6H), 7.03-6.97 (m, 5H), 6.88-6.65 (m, 3H), 6.62 (m, 4H), 3.80(m, 4H), 3.77(m, 4H), 2.93-2.85 (m, 2H), 1.94-1.72 (m, 4H), 1.00 (s, 6H).

### Synthesis of a Compound CPD123

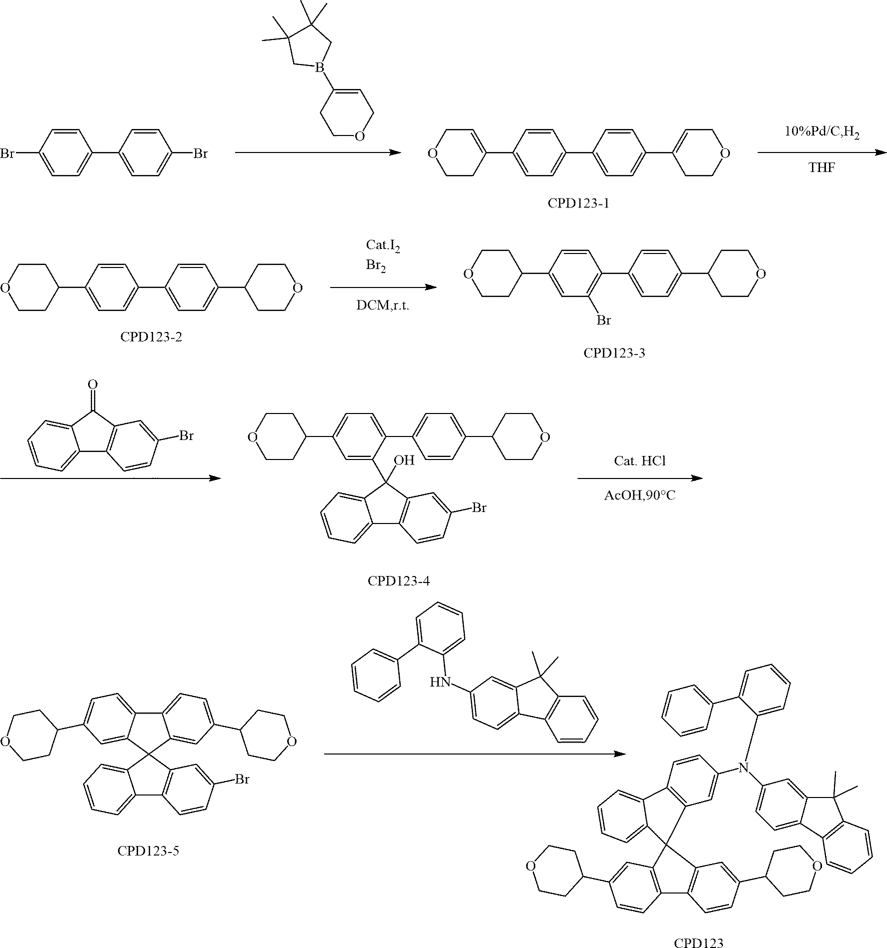

### Synthesis of a Compound CPD123-1

With reference to the synthesis and purification methods of the compound CPD001-1, only the corresponding raw materials were required to be changed, and a target compound CPD123-1 (22.10 g, purity: 99.42%, and yield: 90.21%) was obtained. The mass spectrum was 319.25 (M+H).

### Synthesis of a Compound CPD123-2

With reference to the synthesis and purification methods of the compound CPD001-2, only the corresponding raw materials were required to be changed, and a target compound CPD123-2 (20.97 g, purity: 99.91%, and yield: 93.71%) was obtained. The mass spectrum was 323.25 (M+H).

### Synthesis of a Compound CPD123-3

With reference to the synthesis and purification methods of the compound CPD001-3, only the corresponding raw materials were required to be changed, and a target compound CPD123-3 (24.42 g, purity: 99.16%, and yield: 93.55%) was obtained. The mass spectrum was 401.01 (M+H).

### Synthesis of a Compound CPD123-4

With reference to the synthesis and purification methods of the compound CPD001-4, only the corresponding raw materials were required to be changed, and a target compound CPD123-4 (22.76 g, purity: 99.00%, and yield: 64.33%) was obtained. The mass spectrum was 579.26 (M+H).

### Synthesis of a Compound CPD123-5

With reference to the synthesis and purification methods of the compound CPD001-5, only the corresponding raw materials were required to be changed, and a target compound CPD123-5 (15.58 g, purity: 99.78%, and yield: 70.62%) was obtained. The mass spectrum was 563.36 (M+H).

### Synthesis of a Compound CPD123

With reference to the synthesis and purification methods of the compound CPD001, only the corresponding raw materials were required to be changed, and a white solid, namely a target compound CPD123 (19.27 g, purity: 99.92%, and yield: 82.56%) was obtained. 19.27 g of the crude product CPD123 was sublimated and purified to obtain a sublimated pure product CPD123 (13.57 g, purity: 99.92%, and yield: 70.44%). The mass spectrum was 867.33 (M+Na).

¹H NMR (400 MHz, CDCl₃) δ 7.72(d, J = 7.61 Hz, 1H), 7.57 (d, J=8.32 Hz, 1H), 7.55 (m, 3H), 7.50-7.24 (m, 7H), 7.23-7.14 (m, 6H), 7.03-6.97 (m, 5H), 6.86-6.62 (m, 6H), 3.74(m, 8H), 2.93-2.85 (m, 2H), 2.48-2.11 (m, 8H), 1.01 (s, 6H).

### Synthesis of a Compound CPD124

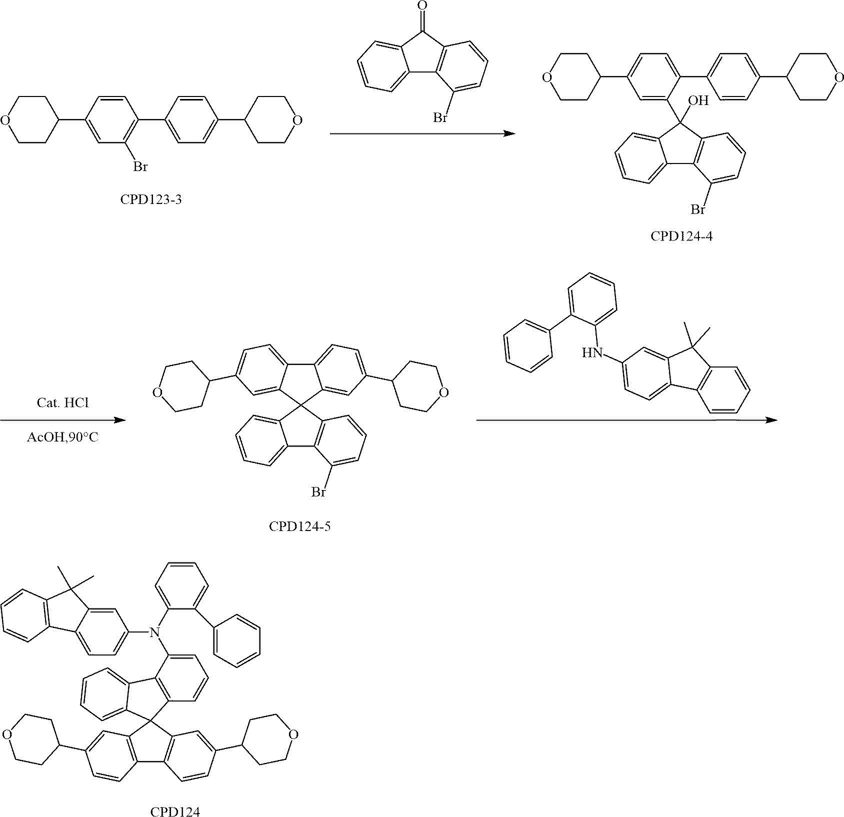

### Synthesis of a Compound CPD 124-4

With reference to the synthesis and purification methods of the compound CPD001-4, only the corresponding raw materials were required to be changed, and a target compound CPD124-4 (23.37 g, purity: 99.10%, and yield: 65.73%) was obtained. The mass spectrum was 579.26 (M+H).

### Synthesis of a Compound CPD124-5

With reference to the synthesis and purification methods of the compound CPD001-5, only the corresponding raw materials were required to be changed, and a target compound CPD124-5 (16.60 g, purity: 99.78%, and yield: 73.30%) was obtained. The mass spectrum was 563.36 (M+H).

### Synthesis of a Compound CPD124

With reference to the synthesis and purification methods of the compound CPD001, only the corresponding raw materials were required to be changed, and a white solid, namely a target compound CPD124 (20.16 g, purity: 99.93%, and yield: 81.07%) was obtained. 20.16 g of the crude product CPD124 was sublimated and purified to obtain a sublimated pure product CPD124 (14.60 g, purity: 99.93%, and yield: 72.43%). The mass spectrum was 867.33 (M+Na).

¹H NMR (400 MHz, CDCl₃) δ 7.71-7.68 (m, 2H), 7.52-7.51(m, 2H), 7.49-7.48 (m, 2H), 7.24-7.13 (m, 4H), 7.06-6.94 (m, 9H), 6.91-6.80 (m, 6H), 6.77-6.60 (m, 4H), 3.74(m, 8H), 2.93-2.85 (m, 2H), 2.48-2.11 (m, 8H), 1.01 (s, 6H).

### APPLICATION EXAMPLE: MANUFACTURE OF AN ORGANIC ELECTROLUMINESCENT DEVICE

A glass substrate with a size of 50 mm*50 mm* 1.0 mm including an ITO (100 nm) transparent electrode was ultrasonically cleaned in ethanol for 10 minutes, dried at 150° C., and then treated with N₂ plasma for 30 minutes. The washed glass substrate was installed on a substrate support of a vacuum evaporation device. At first, a compound HATCN for covering the transparent electrode was evaporated on the surface of the side having a transparent electrode line to form a thin film with a thickness of 5 nm. Next, a layer of HTM1 was evaporated to form a thin film as a hole transport layer 1 (HTL1) with a thickness of 60 nm. Then, a layer of HTM2 was evaporated on the HTM1 thin film to form a thin film as a hole transport layer 2 (HTL2) with a thickness of 10 nm. After that, a main material and a doping material (with a doping proportion of 2%) were co-evaporated on the HTM2 film layer to obtain a film with a thickness of 25 nm, where a ratio of the main material to the doping material was 90%: 10%. A hole blocking layer (HBL, 5 nm) and an electron transport layer (ETL, 30 nm) were evaporated on a light-emitting layer in sequence to serve as a hole blocking layer material and an electron transport material respectively according to combinations in the following table. LiQ (1 nm) was evaporated on the electron transport material layer to serve as an electron injection material. Then, a mixture of Mg and Ag (100 nm, at a ratio of 1:9) was co-evaporated to serve as a cathode material.

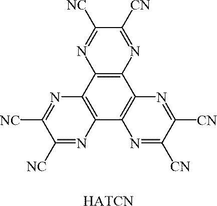

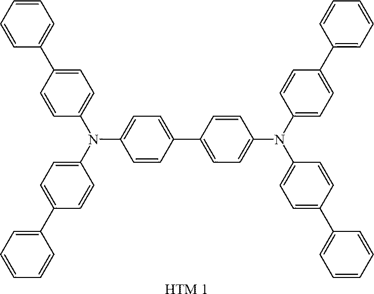

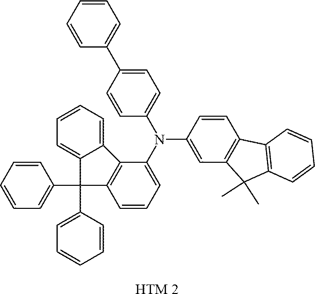

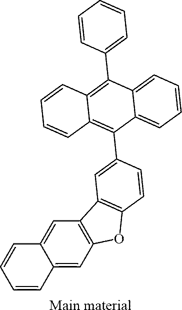

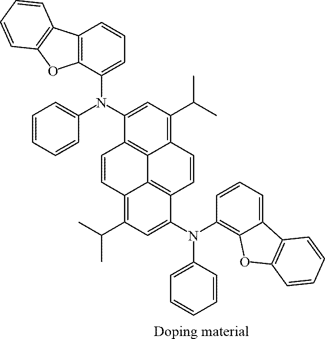

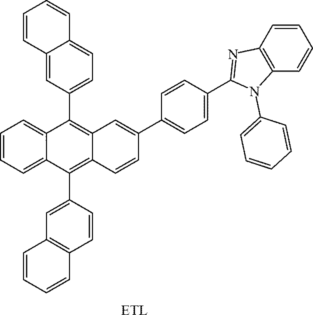

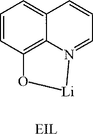

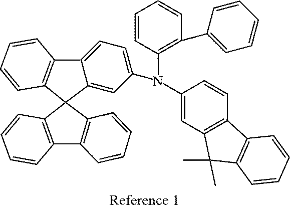

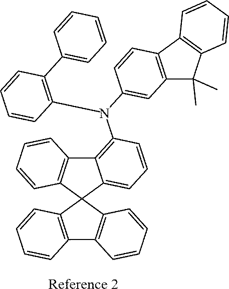

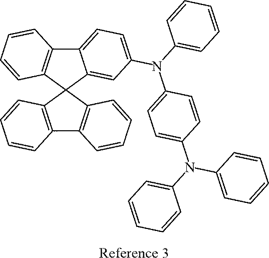

### EVALUATION

Properties of a device obtained above were tested. In the present disclosure, compounds in examples and comparative examples 1-3 were separately used as the HTL for reference, a constant-current power supply (Keithley 2400) was used, a current at a fixed density was used for flowing through light-emitting elements, and a spectroradiometer (CS 2000) was used for testing the light-emitting spectrum. Meanwhile, the voltage value was measured, and the time (LT90) when the brightness was reduced to 90% of an initial brightness was tested. Results are shown in the following Table 1.

| | HTL1 | HTL2 | Starting voltage V | External quantum efficiency (%) | LT90@ |
|---|---|---|---|---|---|
| | | | @ 1000nits | | 1000nits |
| Example 1 | CPD001 | HTM2 | 3.68 | 9.33 | 136 |
| Example 2 | CPD003 | HTM2 | 3.71 | 9.47 | 148 |
| Example 3 | CPD005 | HTM2 | 3.76 | 9.87 | 141 |
| Example 4 | CPD007 | HTM2 | 3.74 | 9.54 | 137 |
| Example 5 | CPD019 | HTM2 | 3.69 | 9.69 | 153 |
| Example 6 | CPD039 | HTM2 | 3.81 | 10.05 | 133 |
| Example 7 | CPD049 | HTM2 | 3.77 | 9.61 | 121 |
| Example 8 | CPD061 | HTM2 | 3.75 | 10.03 | 134 |
| Example 9 | CPD073 | HTM2 | 3.65 | 9.96 | 141 |
| Example 10 | CPD097 | HTM2 | 3.80 | 9.84 | 119 |
| Example 11 | CPD117 | HTM2 | 3.67 | 10.18 | 146 |
| Example 12 | CPD123 | HTM2 | 3.65 | 10.21 | 151 |
| Example 12 | HTM1 | CPD008 | 3.73 | 9.86 | 108 |
| Example 13 | HTM1 | CPD106 | 3.70 | 9.97 | 122 |
| Example 14 | HTM1 | CPD124 | 3.69 | 9.62 | 96 |
| Comparative Example 1 | HTM1 | HTM2 | 3.97 | 8.45 | 35 |
| Comparative Example 2 | Reference 1 | HTM2 | 3.89 | 8.67 | 47 |
| Comparative Example 3 | Reference 2 | HTM2 | 3.96 | 8.87 | 42 |
| Comparative Example 4 | Reference 3 | HTM2 | 3.91 | 9.02 | 64 |
| Comparative Example 5 | HTM1 | Reference 2 | 3.88 | 9.04 | 66 |
| Example 23 | CPD001 | CPD008 | 3.69 | 10.33 | 146 |
| Example 24 | CPD001 | CPD106 | 3.66 | 10.54 | 162 |

Comparison of the sublimation temperature is as follows. The sublimation temperature is defined as the temperature corresponding to an evaporation rate of 1 Å/s at a vacuum degree of 10⁻⁷ Torr. Test results are shown as follows.

| Main material | Sublimation temperature/ °C. |
|---|---|
| CPD001 | 261 |
| CPD003 | 262 |
| CPD005 | 265 |
| Reference compound 1 | 268 |
| Reference compound 2 | 270 |
| Reference compound 3 | 281 |
| HTM1 | 380 |
| HTM2 | 275 |

Through comparison of the data in the above table, it can be seen that the hole transport material of the present disclosure has low sublimation temperature, and industrial application is facilitated.

### COMPARISON OF THE LATERAL MOBILITY OF CARRIERS

A glass substrate with a size of 50 mm*50 mm*1.0 mm was changed to have an ITO (100 nm) transparent electrode and a Mg/Ag (100 nm, 1:9) cathode material at two ends and a groove with a size of 5 mm*5 mm*0.4 mm in the middle. The substrate was ultrasonically cleaned in ethanol for 10 minutes, dried at 150° C., and then treated with N₂ plasma for 30 minutes. The washed glass substrate was installed on a substrate support of a vacuum evaporation device. At first, an HTL1 (the CPD001, reference compounds 1-3, and HTM1 were doped with 3% of HATCN separately) with a film thickness of 10 nm was evaporated on the surface of the side having the transparent electrode by a method of covering the transparent electrode. Then, an HTL2 (which was the CPD001, the reference compounds 1-3, and the HTM1 separately) with a film thickness of 100 nm was evaporated. After encapsulation was conducted, a voltage-current curve was tested to obtain lateral transmission current data. It can be observed that when the voltage is increased to 20 V, the lateral crosstalk current of the CPD001 is the minimum, and is only 2.96*10⁻⁵ mA, which is better than the reference compounds 1-3 and the HTM1. In this way, the lateral mobility of carriers is low, and good gray-scale color purity is facilitated.

| | | |
|---|---|---|
| HTL1 | HTL2 | Transmission current/mA |
| 3% HATCN: 97% CPD001 | CPD001 | 2.96×10⁻⁵ |
| 3% HATCN: 97% reference compound 1 | Reference compound 1 | 3.77×10⁻⁴ |
| 3% HATCN: 97% reference compound 2 | Reference compound 2 | 6.79×10⁻⁴ |
| 3% HATCN: 97% reference compound 3 | Reference compound 3 | 9.36×10⁻⁴ |
| 3% HATCN: 97% HTM1 | HTM1 | 3.01×10⁻³ |

The material of the present disclosure has advantages such as high optical and electrical stability, low sublimation temperature, low drive current, low lateral mobility of carriers, high luminous efficiency, and long service life of a device, and can be used in an organic electroluminescent device. In particular, the compound has the possibility of being applied in the AMOLED industry as a hole injection or transport material.

1. A spiro compound, having a structure as shown in a formula (1), 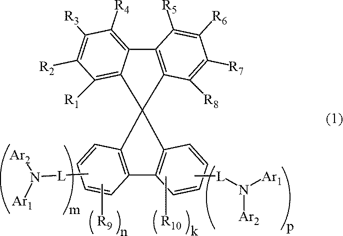
wherein R₁-R₁₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀ heteroalkyl, substituted or unsubstituted C₃-C₂₀ cycloalkyl, substituted or unsubstituted C₃-C₂₀ heterocyclic alkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀ alkyl silyl, substituted or unsubstituted tri-C₆-C₁₂ aryl silyl, substituted or unsubstituted di-C₁-C₁₀ alkyl mono-C₆-C₃₀ aryl silyl, and substituted or unsubstituted mono-Ci-Cio alkyl di-C₆-C₃₀ aryl silyl, or two adjacent groups of R₁-R₈ and R₉-R₁₀ may be connected to each other to form an aliphatic ring or an aromatic ring structure;
at least two groups of the R₁-R₈ are substituted or unsubstituted C₃-C₂₀ cycloalkyl, or substituted or unsubstituted C₃-C₂₀ heterocyclic alkyl;
L is independently selected from a single bond, substituted or unsubstituted C₆-C₃₀ arylene, or substituted or unsubstituted C₂-C₃₀ heteroarylene;
Ar₁ and Ar₂ are independently selected from substituted or unsubstituted C₆-C₃₀ aryl, or substituted or unsubstituted C₂-C₃₀ heteroaryl;
m, n, h, and p are independently selected from 0 or an integer of 1-4, m+n=4, p+k=4, and the m and the p are not 0 at the same time;
the heteroalkyl, the heterocyclic alkyl, and the heteroaryl at least contain one O, N, or S heteroatom; and
the “substituted” refers to substitution with deuterium, F, Cl, Br, C₆-C₁₀ aryl, C₁-C₆ alkyl, C₃-C₆ cycloalkyl, amino substituted with C₁-C₆ alkyl, cyano, isonitrile, or phosphino, and the substitution number ranges from a single substitution number to a maximum substitution number.

2. The spiro compound according to claim 1, wherein m+p=1.

3. The spiro compound according to claim 2, having structures as shown in a formula (2) to a formula (9), 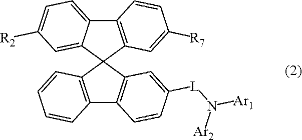 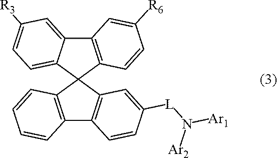 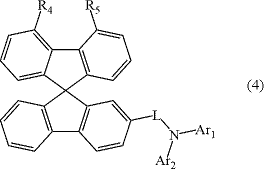 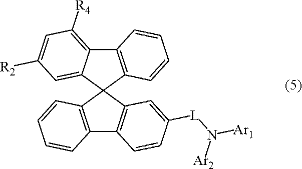 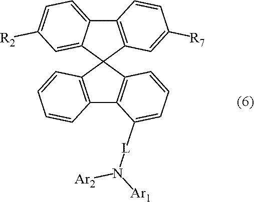 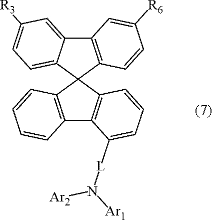 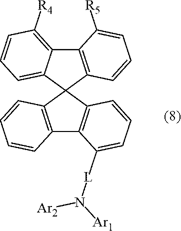 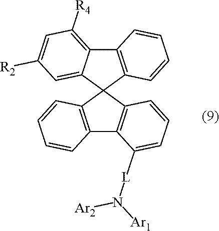
wherein R₂, R₃, R₄, R₅, R₆, and R₇ are substituted or unsubstituted C₃-C₂₀ cycloalkyl, or substituted or unsubstituted C₃-C₂₀ heterocyclic alkyl; and Ar₁, Ar₂, and L are defined the same as above.

4. The spiro compound according to claim 3, having a structure as shown in the formula (2) or formula (6), wherein the R₂ and the R₇ are the same or different, and the Ar₁ and the Ar₂ are the same or different.

5. The spiro compound according to claim 4, wherein the L in the formula (2) to the formula (9) is a single bond.

6. The spiro compound according to claim 5, having structures as shown in a formula (10) to a formula (11), 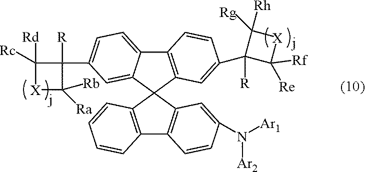 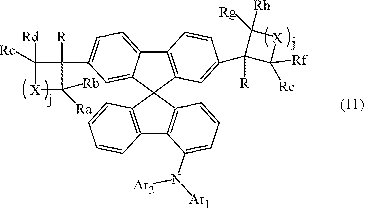
wherein X is independently selected from C(R₀)₂, O, S, and NRo;
j is independently 0 or an integer of 1-7; when the j is equal to 0, a ring formed is a ternary ring; when the j is equal to or greater than 2, various kinds of the X are the same or different;
R, R₀, and Ra-Rh are independently selected from hydrogen, deuterium, halogen, cyano, hydroxyl, sulfhydryl, amino, substituted or unsubstituted C₁-C₁₀ alkyl, substituted or unsubstituted C₁-C₁₀ heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀ alkyl silyl, substituted or unsubstituted tri-C₆-C₁₂ aryl silyl, substituted or unsubstituted di-C₁-C₁₀ alkyl mono-C₆-C₃₀ aryl silyl, and substituted or unsubstituted mono-C₁-C₁₀ alkyl di-C₆-C₃₀ aryl silyl, or four groups of Ra, Rb, Rc, and Rd and/or four groups of Re, Rf, Rg, and Rh and/or various kinds of the R₀ and/or the R and other substituents are connected to each other to form a ring structure; and
the “substituted” refers to substitution with deuterium, F, Cl, Br, C₆-C₁₀ aryl, C₁-C₆ alkyl, C₃-C₆ cycloalkyl, amino substituted with C₁-C₆ alkyl, cyano, isonitrile, or phosphino, and the substitution number ranges from a single substitution number to a maximum substitution number.

7. The spiro compound according to claim 6, wherein the R is hydrogen, deuterium, substituted or unsubstituted C₁-C₁₀ alkyl, or substituted or unsubstituted C₁-C₁₀ heteroalkyl; and
the R₀ and the Ra-Rh are independently selected from hydrogen, deuterium, halogen, substituted or unsubstituted C₁-C₁₀ alkyl, substituted or unsubstituted C₁-C₁₀ heteroalkyl, and substituted or unsubstituted C₃-C₂₀ cycloalkyl, or four groups of the Ra, the Rb, the Rc, and the Rd and/or four groups of the Re, the Rf, the Rg, and the Rh and/or various kinds of the R₀ are connected to each other to form a ring structure.

8. The spiro compound according to claim 7, wherein the j is a value equal to or greater than 2.

9. The spiro compound according to claim 8, wherein at most one of 2 or more of the X is one of O, S, Se, and NR₀.

10. The spiro compound according to any one of claims 5-9, wherein various kinds of the R₀ and/or the R and the R₀ are connected to each other to form a ring structure.

11. The spiro compound according to claim 10, wherein the R₂ and the R₇ are the same, and the Ar₁ and the Ar₂ are different; and the Ar₁ and the Ar₂ are independently selected from substituted or unsubstituted phenyl, biphenyl, naphthyl, fluorenyl, dibenzofuranyl, or carbazolyl, and the “substituted” refers to substitution with deuterium, F, Cl, Br, C₆-C₁₀ aryl, C₁-C₆ alkyl, or C₃-C₆ cycloalkyl.

12. The spiro compound according to claim 1, wherein the spiro compound has one of the following structural formulas, or is partially or completely deuterated or fluorinated correspondingly, 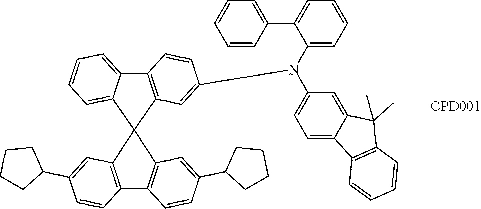 [SMILES: CC1(C)c2ccccc2-c2ccc(N(c3ccc4c(c3)C3(c5ccccc5-4)c4cc(C5CCCC5)ccc4-c4ccc(C5CCCC5)cc43)c3ccccc3-c3ccccc3)cc21] 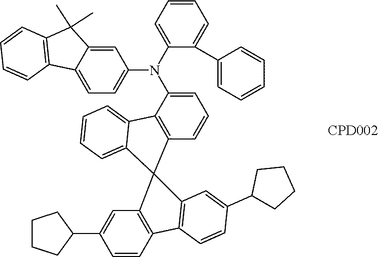 [SMILES: CC1(C)c2ccccc2-c2ccc(N(c3ccccc3-c3ccccc3)c3cccc4c3-c3ccccc3C43c4cc(C5CCCC5)ccc4-c4ccc(C5CCCC5)cc43)cc21] 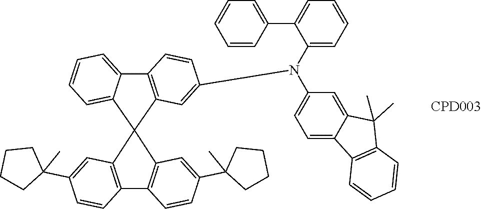 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCCC4)ccc2-3)CCCC1] 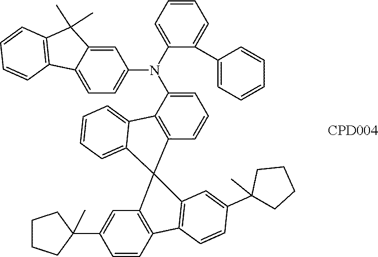 [SMILES: CC1(c2ccc3c(c2)C2(c4cc(C5(C)CCCC5)ccc4-3)c3ccccc3-c3c(N(c4ccc5c(c4)C(C)(C)c4ccccc4-5)c4ccccc4-c4ccccc4)cccc32)CCCC1] 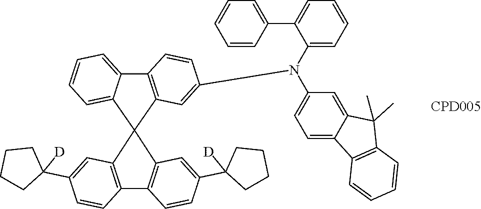 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCCC4)ccc2-3)CCCC1] 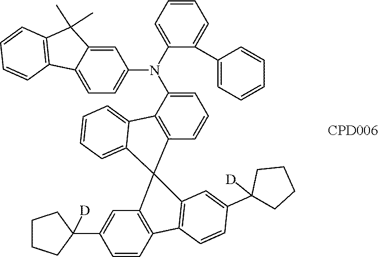 [SMILES: [2H]C1(c2ccc3c(c2)C2(c4cc(C5([2H])CCCC5)ccc4-3)c3ccccc3-c3c(N(c4ccc5c(c4)C(C)(C)c4ccccc4-5)c4ccccc4-c4ccccc4)cccc32)CCCC1] 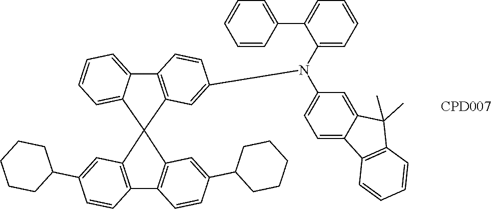 [SMILES: CC1(C)c2ccccc2-c2ccc(N(c3ccc4c(c3)C3(c5ccccc5-4)c4cc(C5CCCCC5)ccc4-c4ccc(C5CCCCC5)cc43)c3ccccc3-c3ccccc3)cc21] 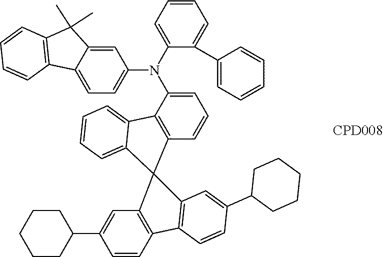 [SMILES: CC1(C)c2ccccc2-c2ccc(N(c3ccccc3-c3ccccc3)c3cccc4c3-c3ccccc3C43c4cc(C5CCCCC5)ccc4-c4ccc(C5CCCCC5)cc43)cc21] 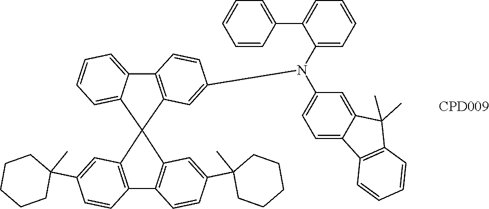 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCCCC4)ccc2-3)CCCCC1] 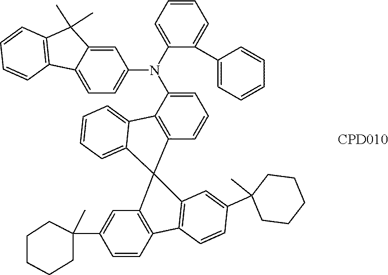 [SMILES: CC1(c2ccc3c(c2)C2(c4cc(C5(C)CCCCC5)ccc4-3)c3ccccc3-c3c(N(c4ccc5c(c4)C(C)(C)c4ccccc4-5)c4ccccc4-c4ccccc4)cccc32)CCCCC1] 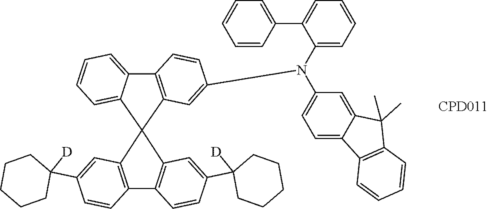 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCCCC4)ccc2-3)CCCCC1] 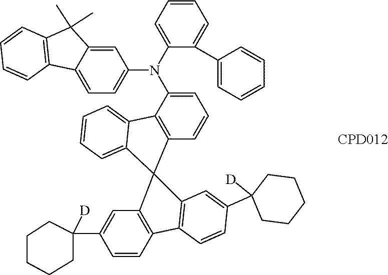 [SMILES: [2H]C1(c2ccc3c(c2)C2(c4cc(C5([2H])CCCCC5)ccc4-3)c3ccccc3-c3c(N(c4ccc5c(c4)C(C)(C)c4ccccc4-5)c4ccccc4-c4ccccc4)cccc32)CCCCC1] 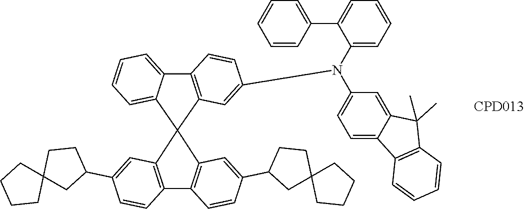 [SMILES: CC1(C)c2ccccc2-c2ccc(N(c3ccc4c(c3)C3(c5ccccc5-4)c4cc(C5CCC6(CCCC6)C5)ccc4-c4ccc(C5CCC6(CCCC6)C5)cc43)c3ccccc3-c3ccccc3)cc21] 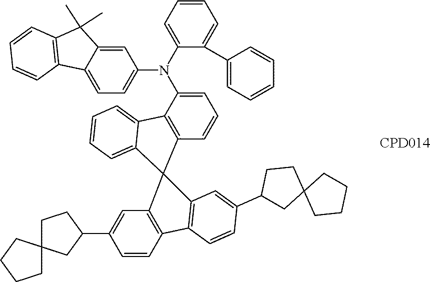 [SMILES: CC1(C)c2ccccc2-c2ccc(N(c3ccccc3-c3ccccc3)c3cccc4c3-c3ccccc3C43c4cc(C5CCC6(CCCC6)C5)ccc4-c4ccc(C5CCC6(CCCC6)C5)cc43)cc21] 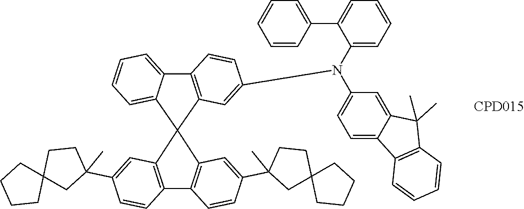 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC5(CCCC5)C4)ccc2-3)CCC2(CCCC2)C1] 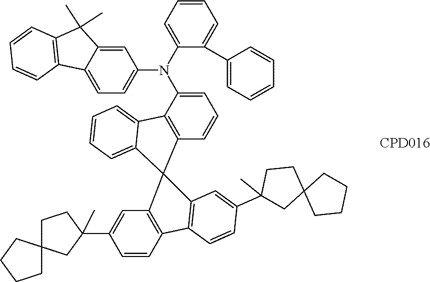 [SMILES: CC1(c2ccc3c(c2)C2(c4cc(C5(C)CCC6(CCCC6)C5)ccc4-3)c3ccccc3-c3c(N(c4ccc5c(c4)C(C)(C)c4ccccc4-5)c4ccccc4-c4ccccc4)cccc32)CCC2(CCCC2)C1] 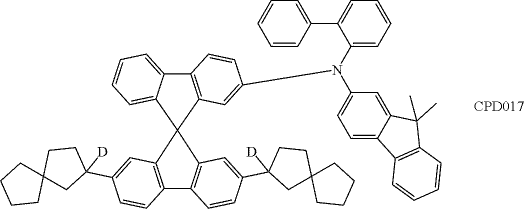 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC5(CCCC5)C4)ccc2-3)CCC2(CCCC2)C1] 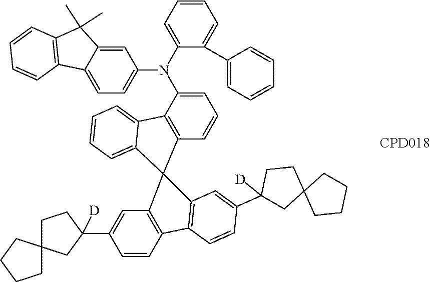 [SMILES: [2H]C1(c2ccc3c(c2)C2(c4cc(C5([2H])CCC6(CCCC6)C5)ccc4-3)c3ccccc3-c3c(N(c4ccc5c(c4)C(C)(C)c4ccccc4-5)c4ccccc4-c4ccccc4)cccc32)CCC2(CCCC2)C1] 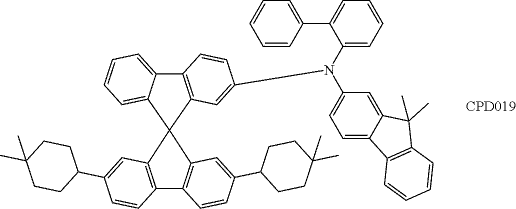 [SMILES: CC1(C)CCC(c2ccc3c(c2)C2(c4ccccc4-c4ccc(N(c5ccc6c(c5)C(C)(C)c5ccccc5-6)c5ccccc5-c5ccccc5)cc42)c2cc(C4CCC(C)(C)CC4)ccc2-3)CC1] 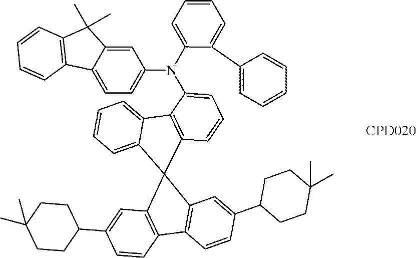 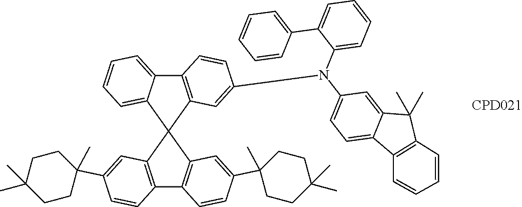 [SMILES: CC1(C)CCC(C)(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC(C)(C)CC4)ccc2-3)CC1] 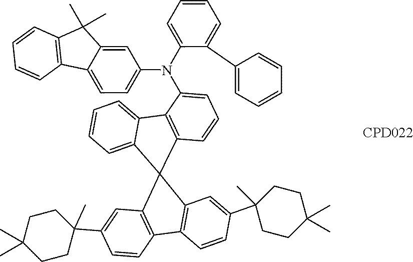 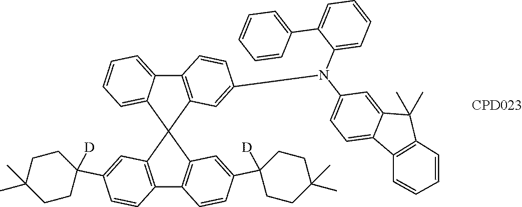 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC(C)(C)CC4)ccc2-3)CCC(C)(C)CC1] 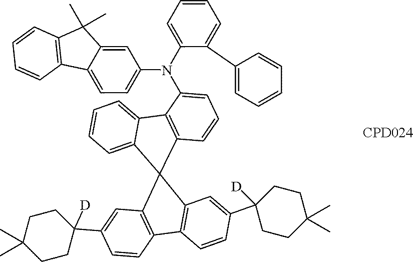 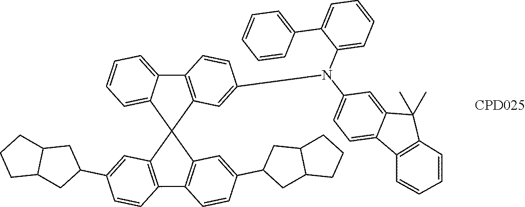 [SMILES: CC1(C)c2ccccc2-c2ccc(N(c3ccc4c(c3)C3(c5ccccc5-4)c4cc(C5CC6CCCC6C5)ccc4-c4ccc(C5CC6CCCC6C5)cc43)c3ccccc3-c3ccccc3)cc21] 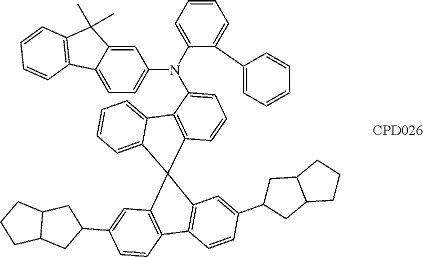 [SMILES: CC1(C)c2ccccc2-c2ccc(N(c3ccccc3-c3ccccc3)c3cccc4c3-c3ccccc3C43c4cc(C5CC6CCCC6C5)ccc4-c4ccc(C5CC6CCCC6C5)cc43)cc21] 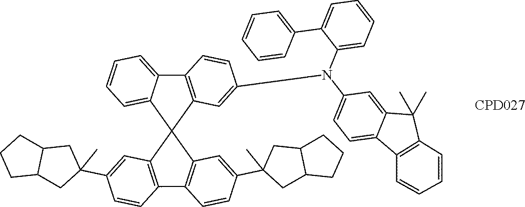 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CC5CCCC5C4)ccc2-3)CC2CCCC2C1] 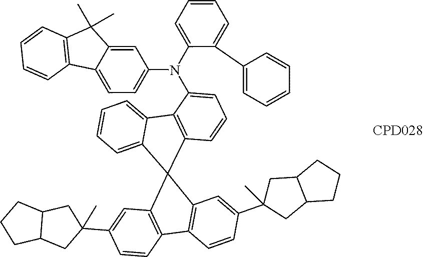 [SMILES: CC1(c2ccc3c(c2)C2(c4cc(C5(C)CC6CCCC6C5)ccc4-3)c3ccccc3-c3c(N(c4ccc5c(c4)C(C)(C)c4ccccc4-5)c4ccccc4-c4ccccc4)cccc32)CC2CCCC2C1] 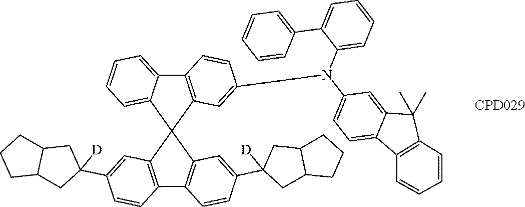 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CC5CCCC5C4)ccc2-3)CC2CCCC2C1] 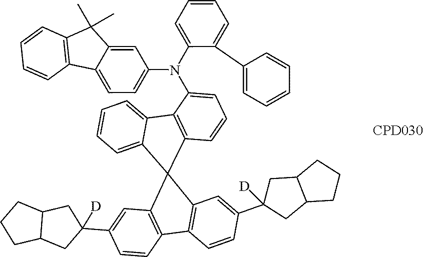 [SMILES: [2H]C1(c2ccc3c(c2)C2(c4cc(C5([2H])CC6CCCC6C5)ccc4-3)c3ccccc3-c3c(N(c4ccc5c(c4)C(C)(C)c4ccccc4-5)c4ccccc4-c4ccccc4)cccc32)CC2CCCC2C1] 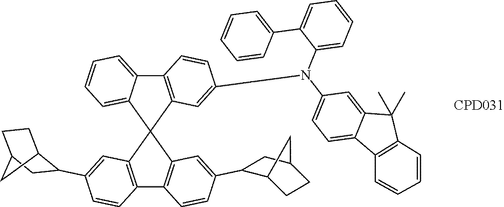 [SMILES: CC1(C)c2ccccc2-c2ccc(N(c3ccc4c(c3)C3(c5ccccc5-4)c4cc(C5CC6CCC5C6)ccc4-c4ccc(C5CC6CCC5C6)cc43)c3ccccc3-c3ccccc3)cc21] 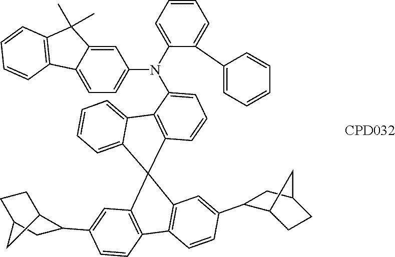 [SMILES: CC1(C)c2ccccc2-c2ccc(N(c3ccccc3-c3ccccc3)c3cccc4c3-c3ccccc3C43c4cc(C5CC6CCC5C6)ccc4-c4ccc(C5CC6CCC5C6)cc43)cc21] 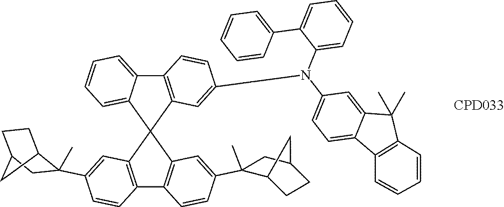 [SMILES: CC1(C)c2ccccc2-c2ccc(N(c3ccc4c(c3)C3(c5ccccc5-4)c4cc(C5(C)CC6CCC5C6)ccc4-c4ccc(C5(C)CC6CCC5C6)cc43)c3ccccc3-c3ccccc3)cc21] 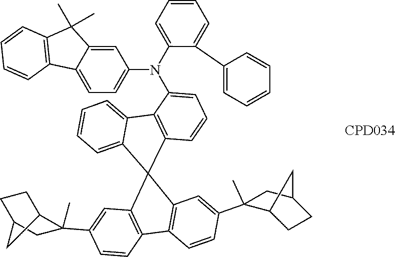 [SMILES: CC1(C)c2ccccc2-c2ccc(N(c3ccccc3-c3ccccc3)c3cccc4c3-c3ccccc3C43c4cc(C5(C)CC6CCC5C6)ccc4-c4ccc(C5(C)CC6CCC5C6)cc43)cc21] 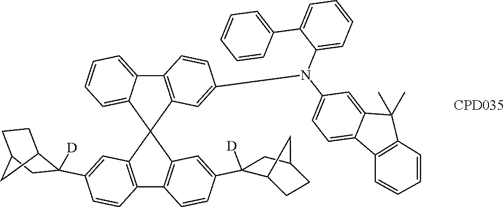 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CC5CCC4C5)ccc2-3)CC2CCC1C2] 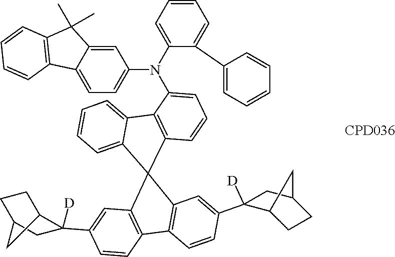 [SMILES: [2H]C1(c2ccc3c(c2)C2(c4cc(C5([2H])CC6CCC5C6)ccc4-3)c3ccccc3-c3c(N(c4ccc5c(c4)C(C)(C)c4ccccc4-5)c4ccccc4-c4ccccc4)cccc32)CC2CCC1C2] 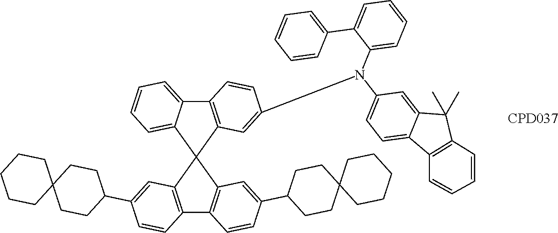 [SMILES: CC1(C)c2ccccc2-c2ccc(N(c3ccc4c(c3)C3(c5ccccc5-4)c4cc(C5CCC6(CCCCC6)CC5)ccc4-c4ccc(C5CCC6(CCCCC6)CC5)cc43)c3ccccc3-c3ccccc3)cc21] 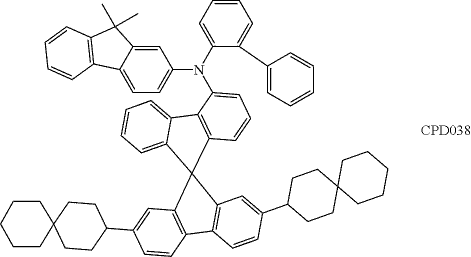 [SMILES: CC1(C)c2ccccc2-c2ccc(N(c3ccccc3-c3ccccc3)c3cccc4c3-c3ccccc3C43c4cc(C5CCC6(CCCCC6)CC5)ccc4-c4ccc(C5CCC6(CCCCC6)CC5)cc43)cc21] 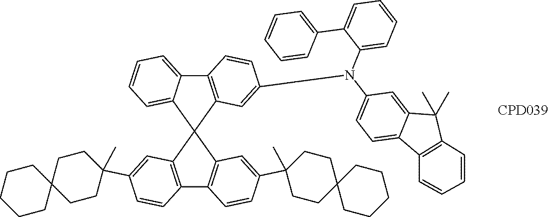 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCC5(CCCCC5)CC4)ccc2-3)CCC2(CCCCC2)CC1] 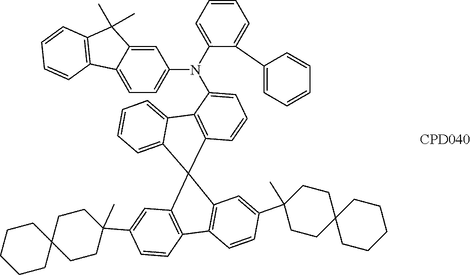 [SMILES: CC1(c2ccc3c(c2)C2(c4cc(C5(C)CCC6(CCCCC6)CC5)ccc4-3)c3ccccc3-c3c(N(c4ccc5c(c4)C(C)(C)c4ccccc4-5)c4ccccc4-c4ccccc4)cccc32)CCC2(CCCCC2)CC1] 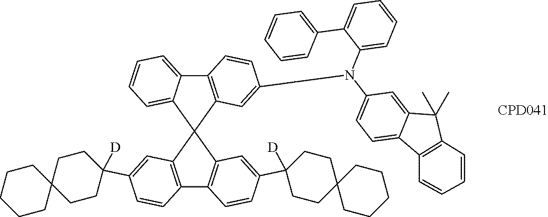 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCC5(CCCCC5)CC4)ccc2-3)CCC2(CCCCC2)CC1] 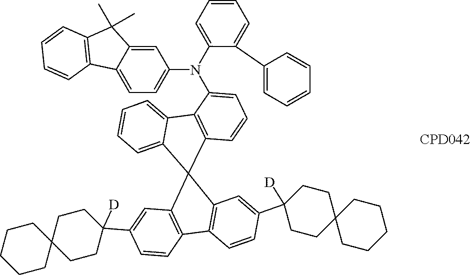 [SMILES: [2H]C1(c2ccc3c(c2)C2(c4cc(C5([2H])CCC6(CCCCC6)CC5)ccc4-3)c3ccccc3-c3c(N(c4ccc5c(c4)C(C)(C)c4ccccc4-5)c4ccccc4-c4ccccc4)cccc32)CCC2(CCCCC2)CC1] 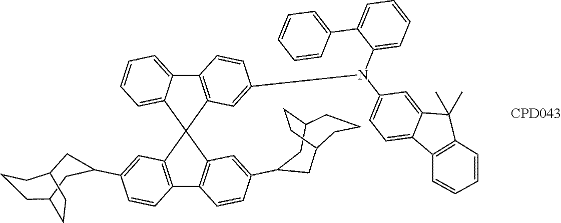 [SMILES: CC1(C)c2ccccc2-c2ccc(N(c3ccc4c(c3)C3(c5ccccc5-4)c4cc(C5CC6CCCC(CC6)C5)ccc4-c4ccc(C5CC6CCCC(CC6)C5)cc43)c3ccccc3-c3ccccc3)cc21] 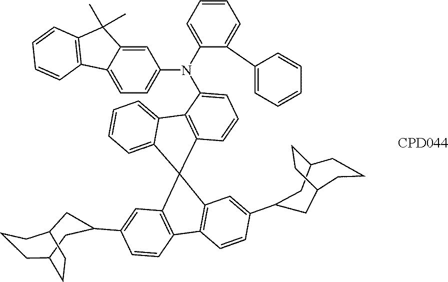 [SMILES: CC1(C)c2ccccc2-c2ccc(N(c3ccccc3-c3ccccc3)c3cccc4c3-c3ccccc3C43c4cc(C5CC6CCCC(CC6)C5)ccc4-c4ccc(C5CC6CCCC(CC6)C5)cc43)cc21] 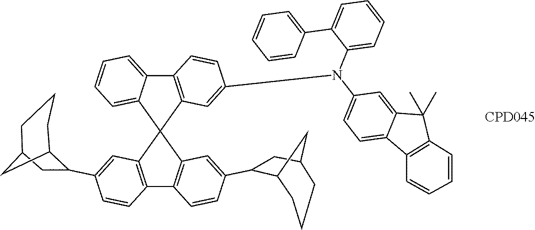 [SMILES: CC1(C)c2ccccc2-c2ccc(N(c3ccc4c(c3)C3(c5ccccc5-4)c4cc(C5CC6CCCC5C6)ccc4-c4ccc(C5CC6CCCC5C6)cc43)c3ccccc3-c3ccccc3)cc21] 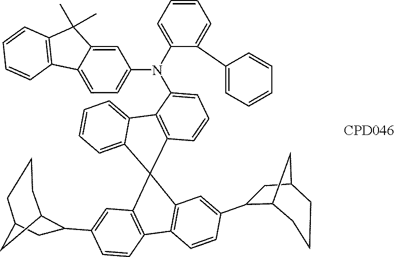 [SMILES: CC1(C)c2ccccc2-c2ccc(N(c3ccccc3-c3ccccc3)c3cccc4c3-c3ccccc3C43c4cc(C5CC6CCCC5C6)ccc4-c4ccc(C5CC6CCCC5C6)cc43)cc21] 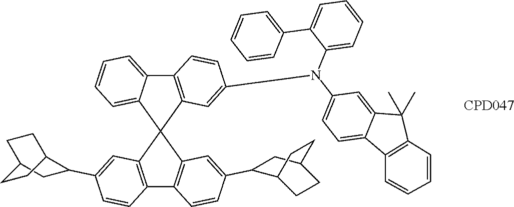 [SMILES: CC1(C)c2ccccc2-c2ccc(N(c3ccc4c(c3)C3(c5ccccc5-4)c4cc(C5CC6CCC5CC6)ccc4-c4ccc(C5CC6CCC5CC6)cc43)c3ccccc3-c3ccccc3)cc21] 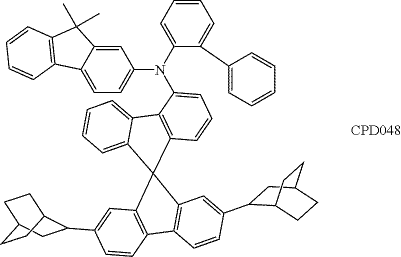 [SMILES: CC1(C)c2ccccc2-c2ccc(N(c3ccccc3-c3ccccc3)c3cccc4c3-c3ccccc3C43c4cc(C5CC6CCC5CC6)ccc4-c4ccc(C5CC6CCC5CC6)cc43)cc21] 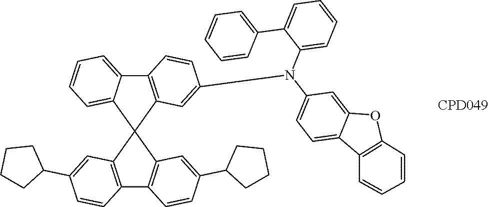 [SMILES: c1ccc(-c2ccccc2N(c2ccc3c(c2)C2(c4ccccc4-3)c3cc(C4CCCC4)ccc3-c3ccc(C4CCCC4)cc32)c2ccc3c(c2)oc2ccccc23)cc1] 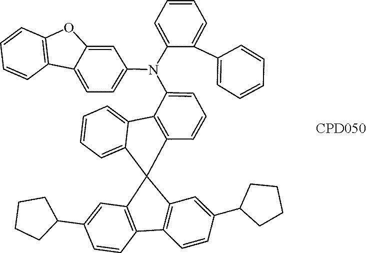 [SMILES: c1ccc(-c2ccccc2N(c2ccc3c(c2)oc2ccccc23)c2cccc3c2-c2ccccc2C32c3cc(C4CCCC4)ccc3-c3ccc(C4CCCC4)cc32)cc1] 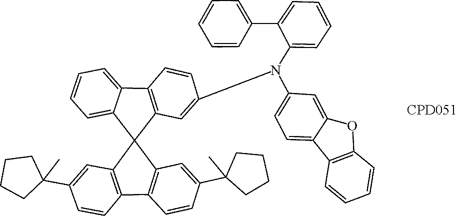 [SMILES: CC1(c2ccc3c(c2)C2(c4ccccc4-c4ccc(N(c5ccc6c(c5)oc5ccccc56)c5ccccc5-c5ccccc5)cc42)c2cc(C4(C)CCCC4)ccc2-3)CCCC1] 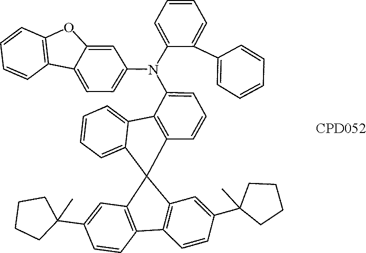 [SMILES: CC1(c2ccc3c(c2)C2(c4cc(C5(C)CCCC5)ccc4-3)c3ccccc3-c3c(N(c4ccc5c(c4)oc4ccccc45)c4ccccc4-c4ccccc4)cccc32)CCCC1] 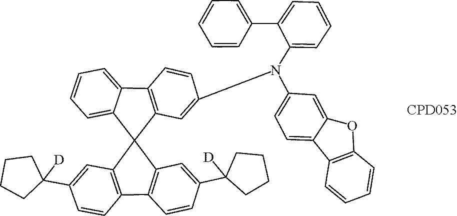 [SMILES: [2H]C1(c2ccc3c(c2)C2(c4ccccc4-c4ccc(N(c5ccc6c(c5)oc5ccccc56)c5ccccc5-c5ccccc5)cc42)c2cc(C4([2H])CCCC4)ccc2-3)CCCC1] 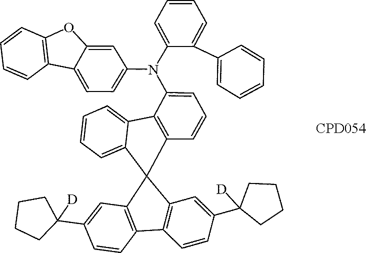 [SMILES: [2H]C1(c2ccc3c(c2)C2(c4cc(C5([2H])CCCC5)ccc4-3)c3ccccc3-c3c(N(c4ccc5c(c4)oc4ccccc45)c4ccccc4-c4ccccc4)cccc32)CCCC1] 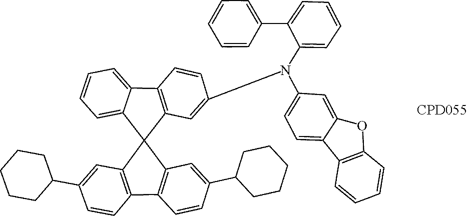 [SMILES: c1ccc(-c2ccccc2N(c2ccc3c(c2)C2(c4ccccc4-3)c3cc(C4CCCCC4)ccc3-c3ccc(C4CCCCC4)cc32)c2ccc3c(c2)oc2ccccc23)cc1] 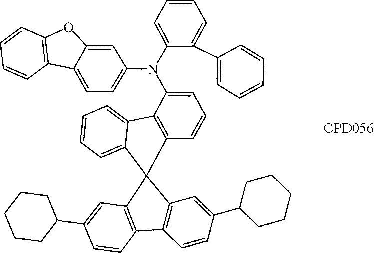 [SMILES: c1ccc(-c2ccccc2N(c2ccc3c(c2)oc2ccccc23)c2cccc3c2-c2ccccc2C32c3cc(C4CCCCC4)ccc3-c3ccc(C4CCCCC4)cc32)cc1] 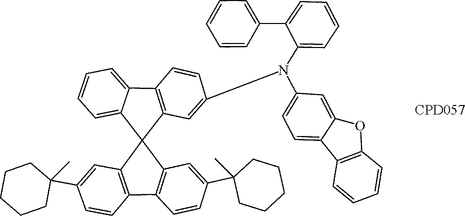 [SMILES: CC1(c2ccc3c(c2)C2(c4ccccc4-c4ccc(N(c5ccc6c(c5)oc5ccccc56)c5ccccc5-c5ccccc5)cc42)c2cc(C4(C)CCCCC4)ccc2-3)CCCCC1] 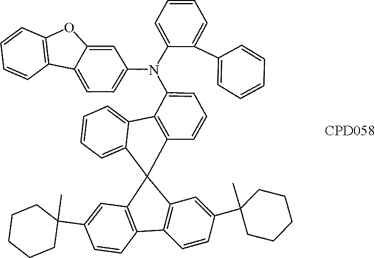 [SMILES: CC1(c2ccc3c(c2)C2(c4cc(C5(C)CCCCC5)ccc4-3)c3ccccc3-c3c(N(c4ccc5c(c4)oc4ccccc45)c4ccccc4-c4ccccc4)cccc32)CCCCC1] 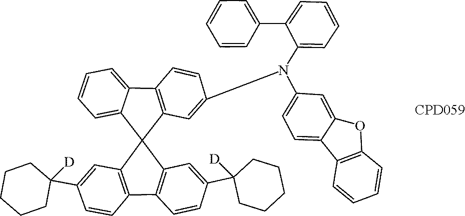 [SMILES: [2H]C1(c2ccc3c(c2)C2(c4ccccc4-c4ccc(N(c5ccc6c(c5)oc5ccccc56)c5ccccc5-c5ccccc5)cc42)c2cc(C4([2H])CCCCC4)ccc2-3)CCCCC1] 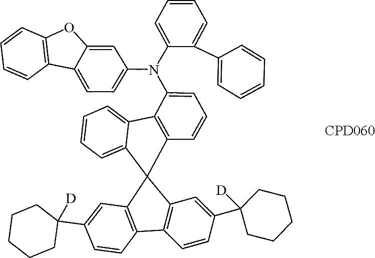 [SMILES: [2H]C1(c2ccc3c(c2)C2(c4cc(C5([2H])CCCCC5)ccc4-3)c3ccccc3-c3c(N(c4ccc5c(c4)oc4ccccc45)c4ccccc4-c4ccccc4)cccc32)CCCCC1] 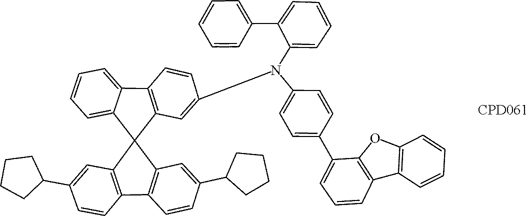 [SMILES: c1ccc(-c2ccccc2N(c2ccc(-c3cccc4c3oc3ccccc34)cc2)c2ccc3c(c2)C2(c4ccccc4-3)c3cc(C4CCCC4)ccc3-c3ccc(C4CCCC4)cc32)cc1] 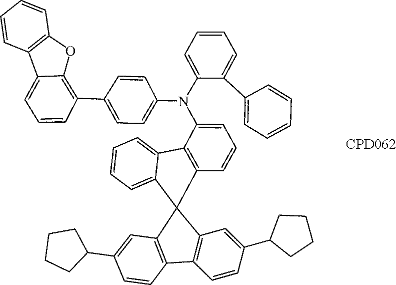 [SMILES: c1ccc(-c2ccccc2N(c2ccc(-c3cccc4c3oc3ccccc34)cc2)c2cccc3c2-c2ccccc2C32c3cc(C4CCCC4)ccc3-c3ccc(C4CCCC4)cc32)cc1] 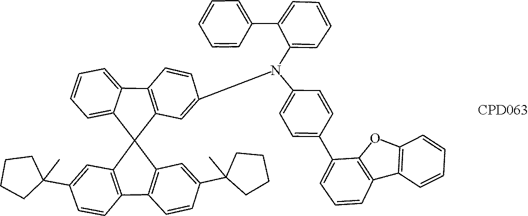 [SMILES: CC1(c2ccc3c(c2)C2(c4ccccc4-c4ccc(N(c5ccc(-c6cccc7c6oc6ccccc67)cc5)c5ccccc5-c5ccccc5)cc42)c2cc(C4(C)CCCC4)ccc2-3)CCCC1] 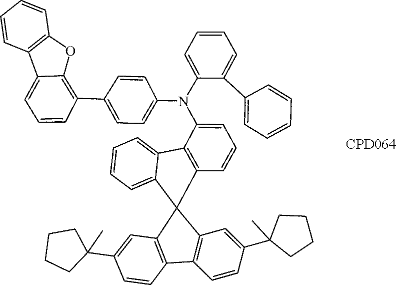 [SMILES: CC1(c2ccc3c(c2)C2(c4cc(C5(C)CCCC5)ccc4-3)c3ccccc3-c3c(N(c4ccc(-c5cccc6c5oc5ccccc56)cc4)c4ccccc4-c4ccccc4)cccc32)CCCC1] 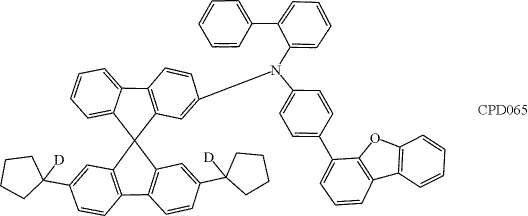 [SMILES: [2H]C1(c2ccc3c(c2)C2(c4ccccc4-c4ccc(N(c5ccc(-c6cccc7c6oc6ccccc67)cc5)c5ccccc5-c5ccccc5)cc42)c2cc(C4([2H])CCCC4)ccc2-3)CCCC1] 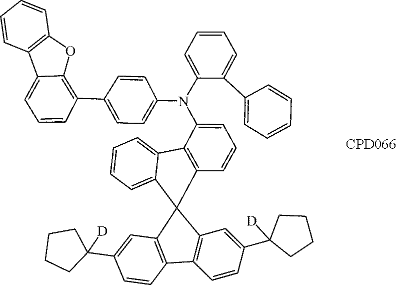 [SMILES: [2H]C1(c2ccc3c(c2)C2(c4cc(C5([2H])CCCC5)ccc4-3)c3ccccc3-c3c(N(c4ccc(-c5cccc6c5oc5ccccc56)cc4)c4ccccc4-c4ccccc4)cccc32)CCCC1] 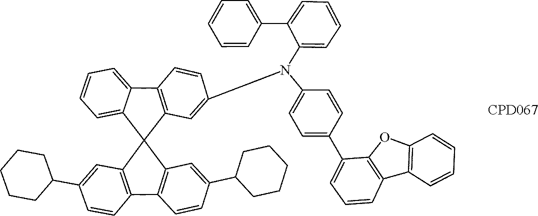 [SMILES: c1ccc(-c2ccccc2N(c2ccc(-c3cccc4c3oc3ccccc34)cc2)c2ccc3c(c2)C2(c4ccccc4-3)c3cc(C4CCCCC4)ccc3-c3ccc(C4CCCCC4)cc32)cc1] 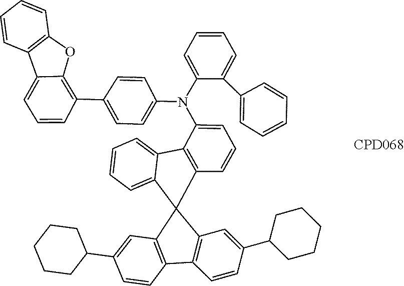 [SMILES: c1ccc(-c2ccccc2N(c2ccc(-c3cccc4c3oc3ccccc34)cc2)c2cccc3c2-c2ccccc2C32c3cc(C4CCCCC4)ccc3-c3ccc(C4CCCCC4)cc32)cc1] 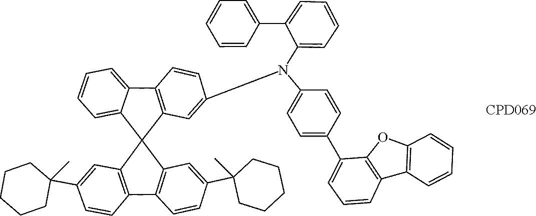 [SMILES: CC1(c2ccc3c(c2)C2(c4ccccc4-c4ccc(N(c5ccc(-c6cccc7c6oc6ccccc67)cc5)c5ccccc5-c5ccccc5)cc42)c2cc(C4(C)CCCCC4)ccc2-3)CCCCC1] 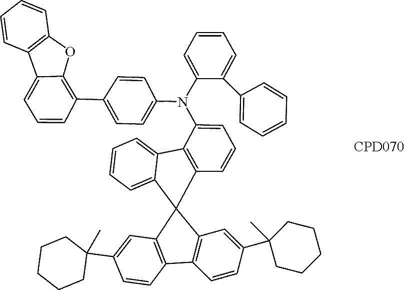 [SMILES: CC1(c2ccc3c(c2)C2(c4cc(C5(C)CCCCC5)ccc4-3)c3ccccc3-c3c(N(c4ccc(-c5cccc6c5oc5ccccc56)cc4)c4ccccc4-c4ccccc4)cccc32)CCCCC1] 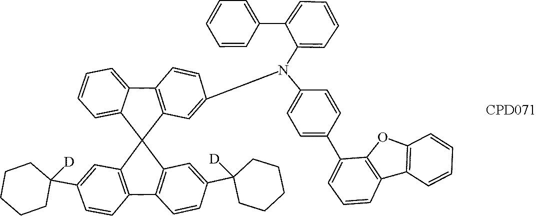 [SMILES: [2H]C1(c2ccc3c(c2)C2(c4ccccc4-c4ccc(N(c5ccc(-c6cccc7c6oc6ccccc67)cc5)c5ccccc5-c5ccccc5)cc42)c2cc(C4([2H])CCCCC4)ccc2-3)CCCCC1] 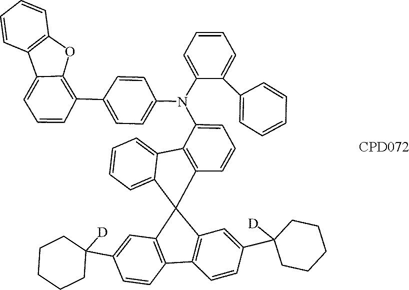 [SMILES: [2H]C1(c2ccc3c(c2)C2(c4cc(C5([2H])CCCCC5)ccc4-3)c3ccccc3-c3c(N(c4ccc(-c5cccc6c5oc5ccccc56)cc4)c4ccccc4-c4ccccc4)cccc32)CCCCC1] 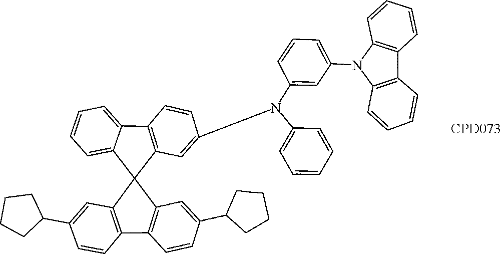 [SMILES: c1ccc(N(c2cccc(-n3c4ccccc4c4ccccc43)c2)c2ccc3c(c2)C2(c4ccccc4-3)c3cc(C4CCCC4)ccc3-c3ccc(C4CCCC4)cc32)cc1] 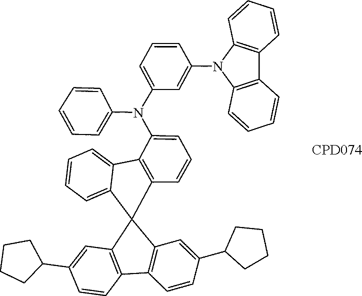 [SMILES: c1ccc(N(c2cccc(-n3c4ccccc4c4ccccc43)c2)c2cccc3c2-c2ccccc2C32c3cc(C4CCCC4)ccc3-c3ccc(C4CCCC4)cc32)cc1] 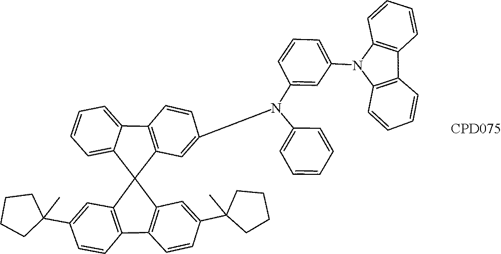 [SMILES: CC1(c2ccc3c(c2)C2(c4ccccc4-c4ccc(N(c5ccccc5)c5cccc(-n6c7ccccc7c7ccccc76)c5)cc42)c2cc(C4(C)CCCC4)ccc2-3)CCCC1] 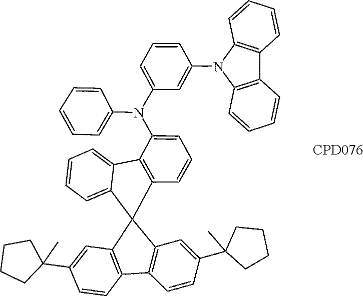 [SMILES: CC1(c2ccc3c(c2)C2(c4cc(C5(C)CCCC5)ccc4-3)c3ccccc3-c3c(N(c4ccccc4)c4cccc(-n5c6ccccc6c6ccccc65)c4)cccc32)CCCC1] 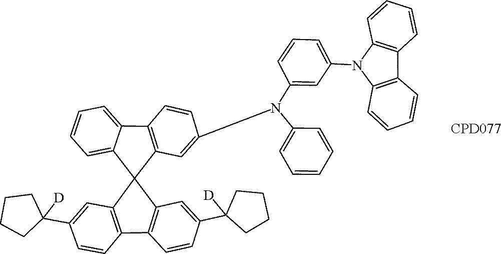 [SMILES: [2H]C1(c2ccc3c(c2)C2(c4ccccc4-c4ccc(N(c5ccccc5)c5cccc(-n6c7ccccc7c7ccccc76)c5)cc42)c2cc(C4([2H])CCCC4)ccc2-3)CCCC1] 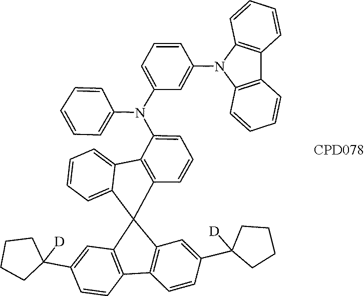 [SMILES: [2H]C1(c2ccc3c(c2)C2(c4cc(C5([2H])CCCC5)ccc4-3)c3ccccc3-c3c(N(c4ccccc4)c4cccc(-n5c6ccccc6c6ccccc65)c4)cccc32)CCCC1] 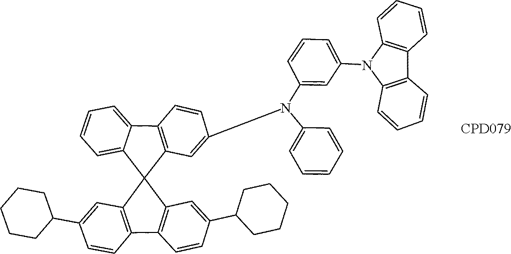 [SMILES: c1ccc(N(c2cccc(-n3c4ccccc4c4ccccc43)c2)c2ccc3c(c2)C2(c4ccccc4-3)c3cc(C4CCCCC4)ccc3-c3ccc(C4CCCCC4)cc32)cc1] 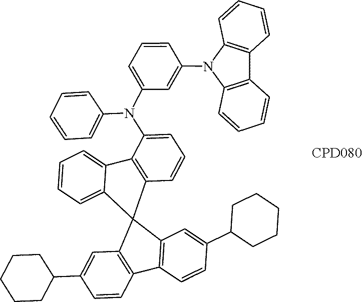 [SMILES: c1ccc(N(c2cccc(-n3c4ccccc4c4ccccc43)c2)c2cccc3c2-c2ccccc2C32c3cc(C4CCCCC4)ccc3-c3ccc(C4CCCCC4)cc32)cc1] 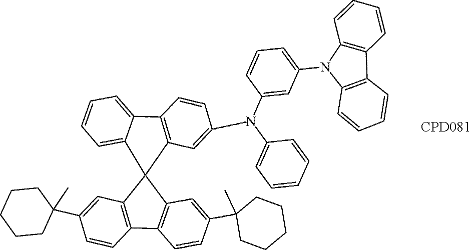 [SMILES: CC1(c2ccc3c(c2)C2(c4ccccc4-c4ccc(N(c5ccccc5)c5cccc(-n6c7ccccc7c7ccccc76)c5)cc42)c2cc(C4(C)CCCCC4)ccc2-3)CCCCC1] 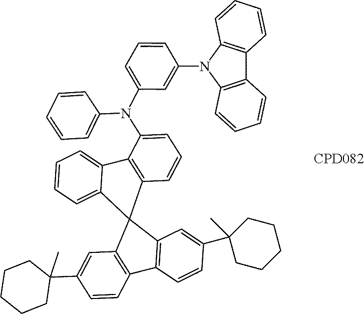 [SMILES: CC1(c2ccc3c(c2)C2(c4cc(C5(C)CCCCC5)ccc4-3)c3ccccc3-c3c(N(c4ccccc4)c4cccc(-n5c6ccccc6c6ccccc65)c4)cccc32)CCCCC1] 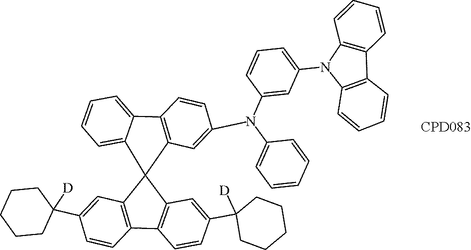 [SMILES: [2H]C1(c2ccc3c(c2)C2(c4ccccc4-c4ccc(N(c5ccccc5)c5cccc(-n6c7ccccc7c7ccccc76)c5)cc42)c2cc(C4([2H])CCCCC4)ccc2-3)CCCCC1] 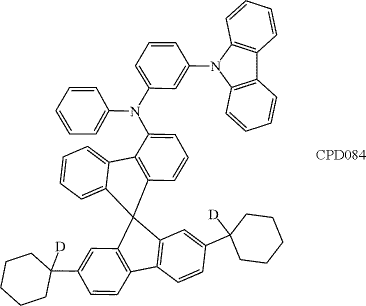 [SMILES: [2H]C1(c2ccc3c(c2)C2(c4cc(C5([2H])CCCCC5)ccc4-3)c3ccccc3-c3c(N(c4ccccc4)c4cccc(-n5c6ccccc6c6ccccc65)c4)cccc32)CCCCC1] 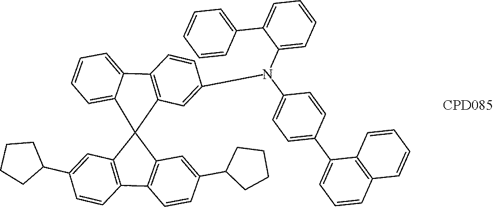 [SMILES: c1ccc(-c2ccccc2N(c2ccc(-c3cccc4ccccc34)cc2)c2ccc3c(c2)C2(c4ccccc4-3)c3cc(C4CCCC4)ccc3-c3ccc(C4CCCC4)cc32)cc1] 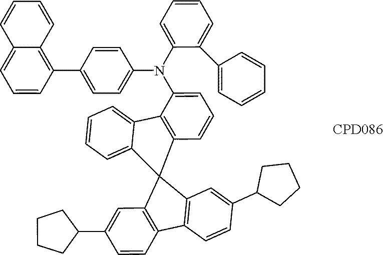 [SMILES: c1ccc(-c2ccccc2N(c2ccc(-c3cccc4ccccc34)cc2)c2cccc3c2-c2ccccc2C32c3cc(C4CCCC4)ccc3-c3ccc(C4CCCC4)cc32)cc1] 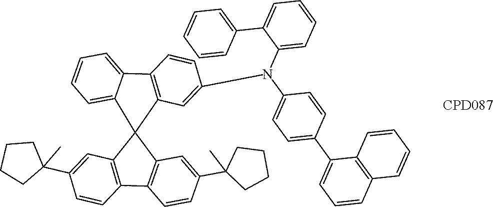 [SMILES: CC1(c2ccc3c(c2)C2(c4ccccc4-c4ccc(N(c5ccc(-c6cccc7ccccc67)cc5)c5ccccc5-c5ccccc5)cc42)c2cc(C4(C)CCCC4)ccc2-3)CCCC1] 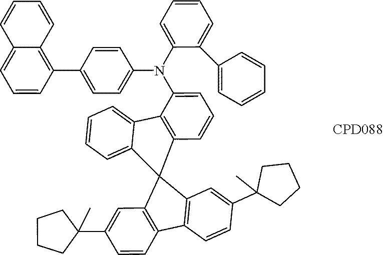 [SMILES: CC1(c2ccc3c(c2)C2(c4cc(C5(C)CCCC5)ccc4-3)c3ccccc3-c3c(N(c4ccc(-c5cccc6ccccc56)cc4)c4ccccc4-c4ccccc4)cccc32)CCCC1] 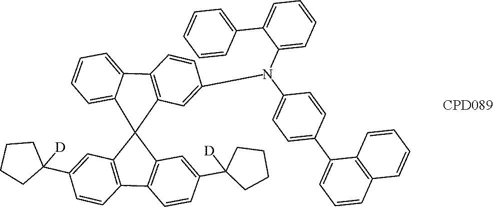 [SMILES: [2H]C1(c2ccc3c(c2)C2(c4ccccc4-c4ccc(N(c5ccc(-c6cccc7ccccc67)cc5)c5ccccc5-c5ccccc5)cc42)c2cc(C4([2H])CCCC4)ccc2-3)CCCC1] 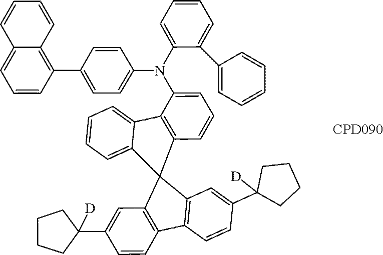 [SMILES: [2H]C1(c2ccc3c(c2)C2(c4cc(C5([2H])CCCC5)ccc4-3)c3ccccc3-c3c(N(c4ccc(-c5cccc6ccccc56)cc4)c4ccccc4-c4ccccc4)cccc32)CCCC1] 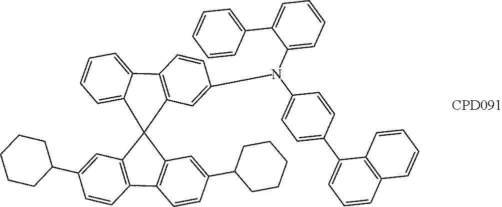 [SMILES: c1ccc(-c2ccccc2N(c2ccc(-c3cccc4ccccc34)cc2)c2ccc3c(c2)C2(c4ccccc4-3)c3cc(C4CCCCC4)ccc3-c3ccc(C4CCCCC4)cc32)cc1] 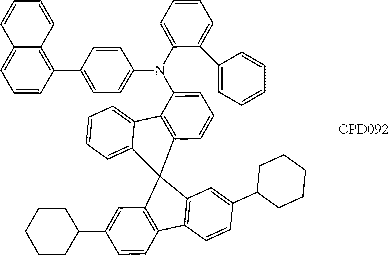 [SMILES: c1ccc(-c2ccccc2N(c2ccc(-c3cccc4ccccc34)cc2)c2cccc3c2-c2ccccc2C32c3cc(C4CCCCC4)ccc3-c3ccc(C4CCCCC4)cc32)cc1] 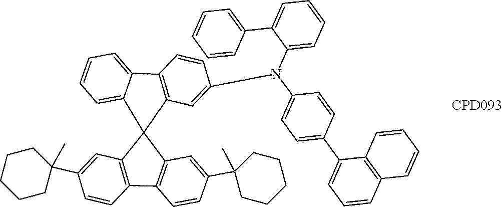 [SMILES: CC1(c2ccc3c(c2)C2(c4ccccc4-c4ccc(N(c5ccc(-c6cccc7ccccc67)cc5)c5ccccc5-c5ccccc5)cc42)c2cc(C4(C)CCCCC4)ccc2-3)CCCCC1] 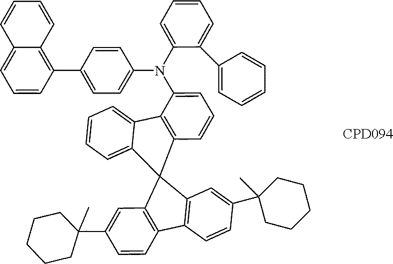 [SMILES: CC1(c2ccc3c(c2)C2(c4cc(C5(C)CCCCC5)ccc4-3)c3ccccc3-c3c(N(c4ccc(-c5cccc6ccccc56)cc4)c4ccccc4-c4ccccc4)cccc32)CCCCC1] 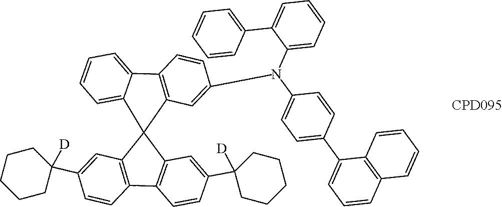 [SMILES: [2H]C1(c2ccc3c(c2)C2(c4ccccc4-c4ccc(N(c5ccc(-c6cccc7ccccc67)cc5)c5ccccc5-c5ccccc5)cc42)c2cc(C4([2H])CCCCC4)ccc2-3)CCCCC1] 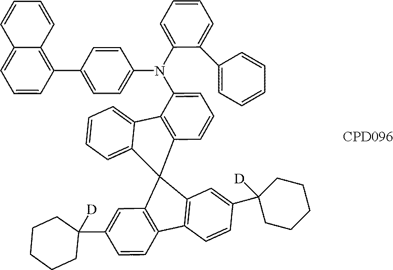 [SMILES: [2H]C1(c2ccc3c(c2)C2(c4cc(C5([2H])CCCCC5)ccc4-3)c3ccccc3-c3c(N(c4ccc(-c5cccc6ccccc56)cc4)c4ccccc4-c4ccccc4)cccc32)CCCCC1] 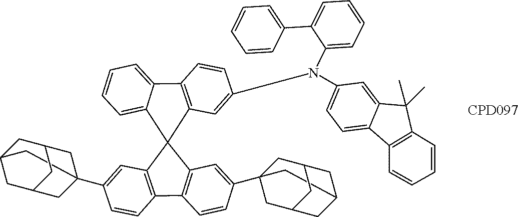 [SMILES: CC1(C)c2ccccc2-c2ccc(N(c3ccc4c(c3)C3(c5ccccc5-4)c4cc(C56CC7CC(CC(C7)C5)C6)ccc4-c4ccc(C56CC7CC(CC(C7)C5)C6)cc43)c3ccccc3-c3ccccc3)cc21] 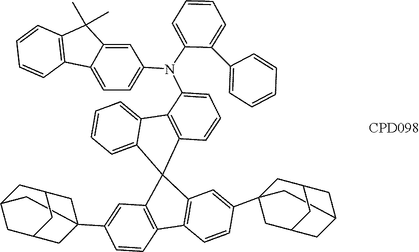 [SMILES: CC1(C)c2ccccc2-c2ccc(N(c3ccccc3-c3ccccc3)c3cccc4c3-c3ccccc3C43c4cc(C56CC7CC(CC(C7)C5)C6)ccc4-c4ccc(C56CC7CC(CC(C7)C5)C6)cc43)cc21] 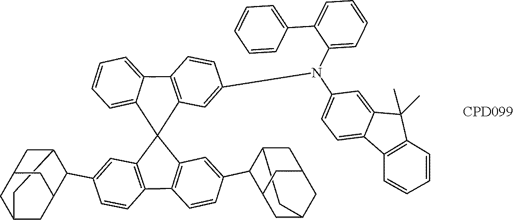 [SMILES: CC1(C)c2ccccc2-c2ccc(N(c3ccc4c(c3)C3(c5ccccc5-4)c4cc(C5C6CC7CC(C6)CC5C7)ccc4-c4ccc(C5C6CC7CC(C6)CC5C7)cc43)c3ccccc3-c3ccccc3)cc21] 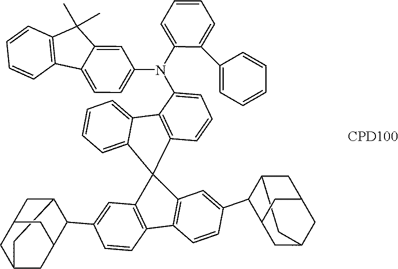 [SMILES: CC1(C)c2ccccc2-c2ccc(N(c3ccccc3-c3ccccc3)c3cccc4c3-c3ccccc3C43c4cc(C5C6CC7CC(C6)CC5C7)ccc4-c4ccc(C5C6CC7CC(C6)CC5C7)cc43)cc21] 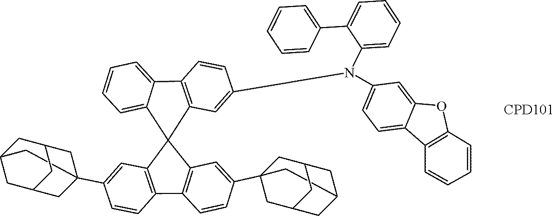 [SMILES: c1ccc(-c2ccccc2N(c2ccc3c(c2)C2(c4ccccc4-3)c3cc(C45CC6CC(CC(C6)C4)C5)ccc3-c3ccc(C45CC6CC(CC(C6)C4)C5)cc32)c2ccc3c(c2)oc2ccccc23)cc1] 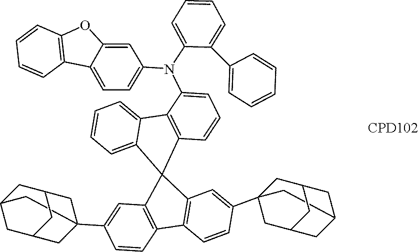 [SMILES: c1ccc(-c2ccccc2N(c2ccc3c(c2)oc2ccccc23)c2cccc3c2-c2ccccc2C32c3cc(C45CC6CC(CC(C6)C4)C5)ccc3-c3ccc(C45CC6CC(CC(C6)C4)C5)cc32)cc1] 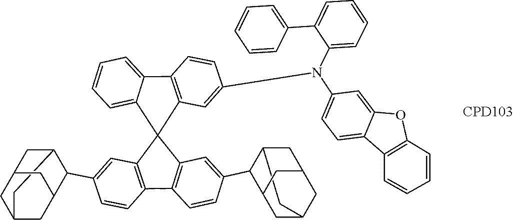 [SMILES: c1ccc(-c2ccccc2N(c2ccc3c(c2)C2(c4ccccc4-3)c3cc(C4C5CC6CC(C5)CC4C6)ccc3-c3ccc(C4C5CC6CC(C5)CC4C6)cc32)c2ccc3c(c2)oc2ccccc23)cc1] 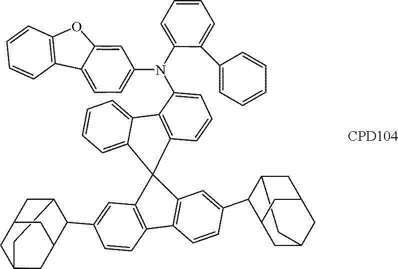 [SMILES: c1ccc(-c2ccccc2N(c2ccc3c(c2)oc2ccccc23)c2cccc3c2-c2ccccc2C32c3cc(C4C5CC6CC(C5)CC4C6)ccc3-c3ccc(C4C5CC6CC(C5)CC4C6)cc32)cc1] 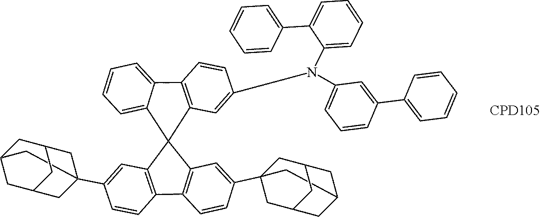 [SMILES: c1ccc(-c2cccc(N(c3ccc4c(c3)C3(c5ccccc5-4)c4cc(C56CC7CC(CC(C7)C5)C6)ccc4-c4ccc(C56CC7CC(CC(C7)C5)C6)cc43)c3ccccc3-c3ccccc3)c2)cc1] 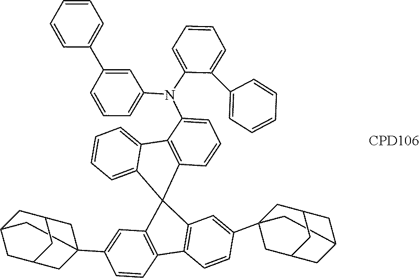 [SMILES: c1ccc(-c2cccc(N(c3ccccc3-c3ccccc3)c3cccc4c3-c3ccccc3C43c4cc(C56CC7CC(CC(C7)C5)C6)ccc4-c4ccc(C56CC7CC(CC(C7)C5)C6)cc43)c2)cc1] 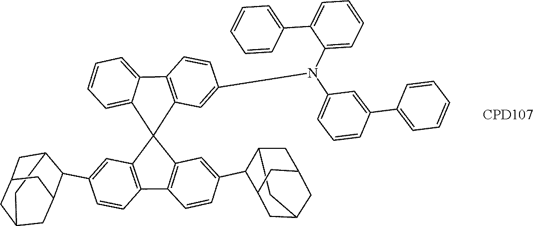 [SMILES: c1ccc(-c2cccc(N(c3ccc4c(c3)C3(c5ccccc5-4)c4cc(C5C6CC7CC(C6)CC5C7)ccc4-c4ccc(C5C6CC7CC(C6)CC5C7)cc43)c3ccccc3-c3ccccc3)c2)cc1] 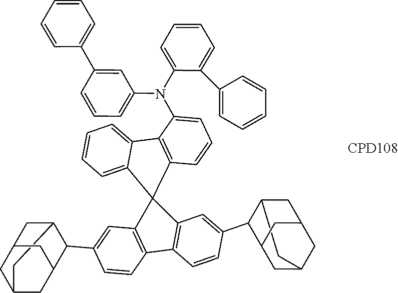 [SMILES: c1ccc(-c2cccc(N(c3ccccc3-c3ccccc3)c3cccc4c3-c3ccccc3C43c4cc(C5C6CC7CC(C6)CC5C7)ccc4-c4ccc(C5C6CC7CC(C6)CC5C7)cc43)c2)cc1] 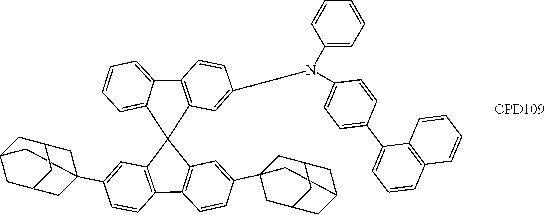 [SMILES: c1ccc(N(c2ccc(-c3cccc4ccccc34)cc2)c2ccc3c(c2)C2(c4ccccc4-3)c3cc(C45CC6CC(CC(C6)C4)C5)ccc3-c3ccc(C45CC6CC(CC(C6)C4)C5)cc32)cc1] 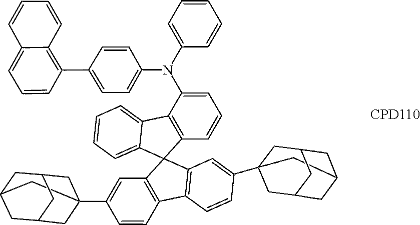 [SMILES: c1ccc(N(c2ccc(-c3cccc4ccccc34)cc2)c2cccc3c2-c2ccccc2C32c3cc(C45CC6CC(CC(C6)C4)C5)ccc3-c3ccc(C45CC6CC(CC(C6)C4)C5)cc32)cc1] 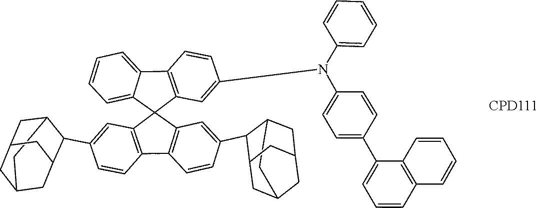 [SMILES: c1ccc(N(c2ccc(-c3cccc4ccccc34)cc2)c2ccc3c(c2)C2(c4ccccc4-3)c3cc(C4C5CC6CC(C5)CC4C6)ccc3-c3ccc(C4C5CC6CC(C5)CC4C6)cc32)cc1] 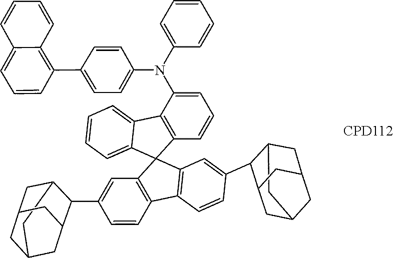 [SMILES: c1ccc(N(c2ccc(-c3cccc4ccccc34)cc2)c2cccc3c2-c2ccccc2C32c3cc(C4C5CC6CC(C5)CC4C6)ccc3-c3ccc(C4C5CC6CC(C5)CC4C6)cc32)cc1] 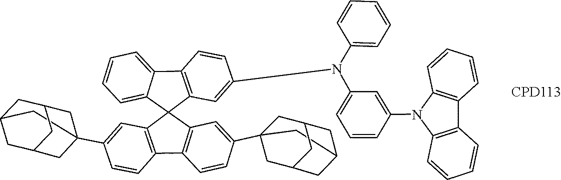 [SMILES: c1ccc(N(c2cccc(-n3c4ccccc4c4ccccc43)c2)c2ccc3c(c2)C2(c4ccccc4-3)c3cc(C45CC6CC(CC(C6)C4)C5)ccc3-c3ccc(C45CC6CC(CC(C6)C4)C5)cc32)cc1] 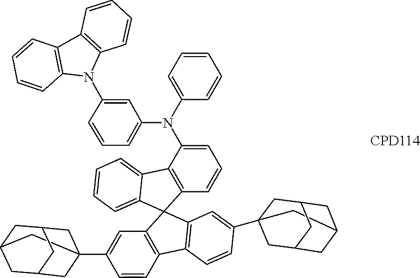 [SMILES: c1ccc(N(c2cccc(-n3c4ccccc4c4ccccc43)c2)c2cccc3c2-c2ccccc2C32c3cc(C45CC6CC(CC(C6)C4)C5)ccc3-c3ccc(C45CC6CC(CC(C6)C4)C5)cc32)cc1] 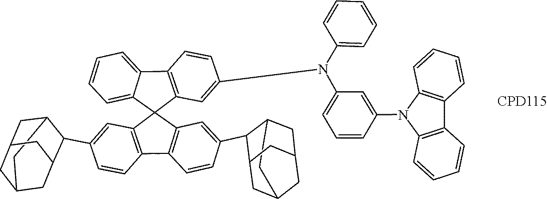 [SMILES: c1ccc(N(c2cccc(-n3c4ccccc4c4ccccc43)c2)c2ccc3c(c2)C2(c4ccccc4-3)c3cc(C4C5CC6CC(C5)CC4C6)ccc3-c3ccc(C4C5CC6CC(C5)CC4C6)cc32)cc1] 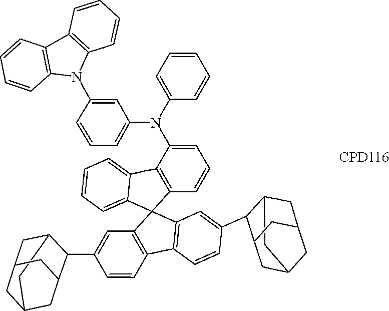 [SMILES: c1ccc(N(c2cccc(-n3c4ccccc4c4ccccc43)c2)c2cccc3c2-c2ccccc2C32c3cc(C4C5CC6CC(C5)CC4C6)ccc3-c3ccc(C4C5CC6CC(C5)CC4C6)cc32)cc1] 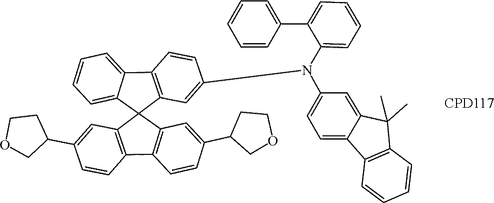 [SMILES: CC1(C)c2ccccc2-c2ccc(N(c3ccc4c(c3)C3(c5ccccc5-4)c4cc(C5CCOC5)ccc4-c4ccc(C5CCOC5)cc43)c3ccccc3-c3ccccc3)cc21] 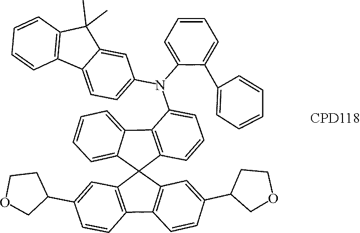 [SMILES: CC1(C)c2ccccc2-c2ccc(N(c3ccccc3-c3ccccc3)c3cccc4c3-c3ccccc3C43c4cc(C5CCOC5)ccc4-c4ccc(C5CCOC5)cc43)cc21] 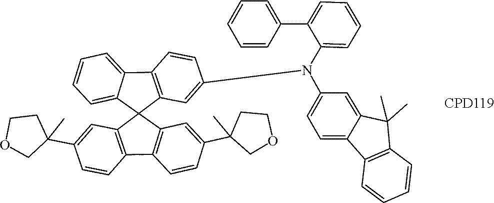 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCOC4)ccc2-3)CCOC1] 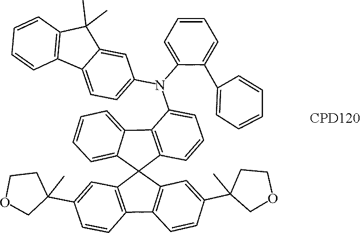 [SMILES: CC1(c2ccc3c(c2)C2(c4cc(C5(C)CCOC5)ccc4-3)c3ccccc3-c3c(N(c4ccc5c(c4)C(C)(C)c4ccccc4-5)c4ccccc4-c4ccccc4)cccc32)CCOC1] 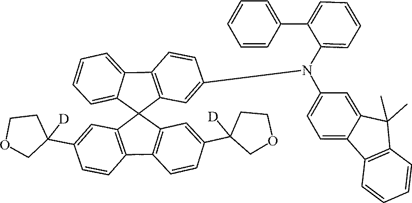 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCOC4)ccc2-3)CCOC1] 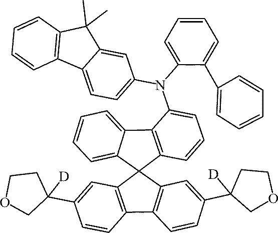 [SMILES: [2H]C1(c2ccc3c(c2)C2(c4cc(C5([2H])CCOC5)ccc4-3)c3ccccc3-c3c(N(c4ccc5c(c4)C(C)(C)c4ccccc4-5)c4ccccc4-c4ccccc4)cccc32)CCOC1] 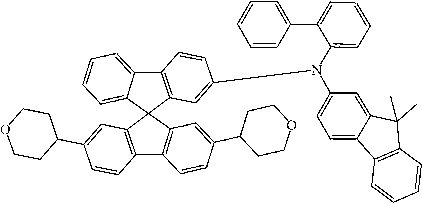 [SMILES: CC1(C)c2ccccc2-c2ccc(N(c3ccc4c(c3)C3(c5ccccc5-4)c4cc(C5CCOCC5)ccc4-c4ccc(C5CCOCC5)cc43)c3ccccc3-c3ccccc3)cc21] 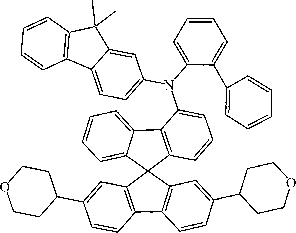 [SMILES: CC1(C)c2ccccc2-c2ccc(N(c3ccccc3-c3ccccc3)c3cccc4c3-c3ccccc3C43c4cc(C5CCOCC5)ccc4-c4ccc(C5CCOCC5)cc43)cc21] 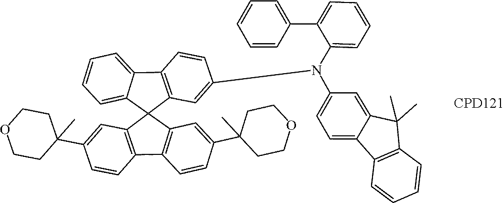 [SMILES: CC1(c2ccc3c(c2)C2(c4ccccc4-c4ccc(N(c5ccc6c(c5)C(C)(C)c5ccccc5-6)c5ccccc5-c5ccccc5)cc42)c2cc(C4(C)CCOCC4)ccc2-3)CCOCC1] 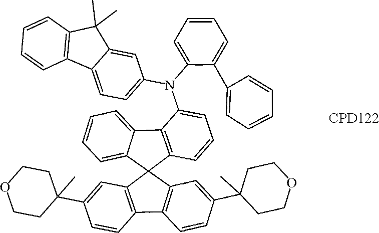 [SMILES: CC1(c2ccc3c(c2)C2(c4cc(C5(C)CCOCC5)ccc4-3)c3ccccc3-c3c(N(c4ccc5c(c4)C(C)(C)c4ccccc4-5)c4ccccc4-c4ccccc4)cccc32)CCOCC1] 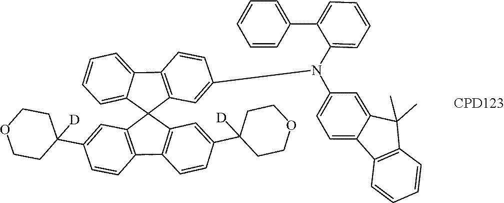 [SMILES: [2H]C1(c2ccc3c(c2)C2(c4ccccc4-c4ccc(N(c5ccc6c(c5)C(C)(C)c5ccccc5-6)c5ccccc5-c5ccccc5)cc42)c2cc(C4([2H])CCOCC4)ccc2-3)CCOCC1] 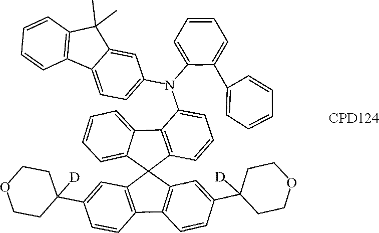 [SMILES: [2H]C1(c2ccc3c(c2)C2(c4cc(C5([2H])CCOCC5)ccc4-3)c3ccccc3-c3c(N(c4ccc5c(c4)C(C)(C)c4ccccc4-5)c4ccccc4-c4ccccc4)cccc32)CCOCC1]
.

13. Application of the spiro compound according to any one of claims 1-12 in an organic electroluminescent device.

14. The application according to claim 13, wherein the spiro compound according to any one of claims 1-12 is used as a material of a hole injection layer and/or a hole transport layer of an organic electroluminescent device.
